(19) United States
(12) Patent Application Publication
(10) Pub. No.: US 2022/0331504 A1
(43) Pub. Date: Oct. 20, 2022

(54) Endovascular Cannula for Defining a Border of a Transport Volume for an In-Vivo Fluid Transport, Cannula System and Corresponding Method

(71) Applicant: ReCO2very Therapies GmbH, Bad Klosterlausnitz (DE)

(72) Inventor: Torsten HEILMANN, Bad Klosterlausnitz (DE)

(21) Appl. No.: 17/635,930

(22) PCT Filed: Aug. 19, 2020
(86) PCT No.: PCT/EP2020/073251

§ 371 Date: Feb. 16, 2022

(30) Foreign Application Priority Data
Aug. 19, 2019 (EP) PCT/EP2019/072166

(51) Int. Cl. A61M 1/36; A61M 60/38; A61M 60/113; A61M 60/37; A61M 25/10; A61M 16/14; A61M 5/142

(52) U.S. Cl. CPC A61M 1/3659; A61M 60/38; A61M 60/113; A61M 60/37; A61M 1/3667; A61M 25/10; A61M 16/14; A61M 5/142; A61M 2205/051

(57) ABSTRACT

Described is an endovascular cannula (L1b, L2b) for defining a border of a transport volume (TrV) for an in-vivo fluid transport, the cannula (L1b, L2b) comprising:—a lumen portion (LP) that extends between a proximal end of the cannula (L1b, L2b) and a distal end of the cannula (L1b, L2b), the lumen portion (LP) defining an inner lumen, and—an expandable arrangement that has a non-expanded state and an expanded state, wherein the expandable arrangement can be switched from the non-expanded state to the expanded state, wherein in the expanded state the expandable arrangement is adapted to define at least one border of the transport volume (TrV), and wherein the border is configured to separate the transport volume from a body fluid circuit (BC).

There are a lot of severe lung diseases that cannot be treated or healed. Even if the thorax is opened by surgery treatment may be difficult. Examples are:
chronic obstructive pulmonary disease (COPD),
acute respiratory distress symptom (ARDS),
pulmonary embolism (PE),
pulmonary hypertension (PHT),
lung fibrosis,
pneumonia, and
cancer, etc.

Usually medicaments or drugs are given or administered to mitigate these diseases. Furthermore ECMO (extracorporeal membrane oxygenation), for instance VA-ECMO (veno-arterial), or ECCO₂R (extracorporeal CO₂ (carbon dioxide) removal) or pECLA (pumpless extracorporeal lung assist) may be performed to mitigate the symptoms. Furthermore, heart assist may be applied, for instance during the treatment of the lung and or during ECMO, ECCO₂R etc.

Furthermore, lung cancer may be treated by a chemotherapy or by chemotherapies. However, it is difficult to take the chemicals to the lung without extensive surgery.

In particular, the patient may suffer from a disease of the lung selected from the group consisting of or comprising cancer, COPS, ARDS and an infectious disease, for instance pneumonia. However, diseases of other organs are also contemplated in the following, especially diseases that are similar to the diseases that are mentioned above. Furthermore, the body is a complex system of organs and a disease or healing of a disease of one organ may have an impact on another organ.

Thus, there is an urgent need to improve treatment of these diseases and of other diseases. Furthermore, there seems to open up a new multi-billion EURO market that may be referred to as a blue ocean in marketing of products and services.

Therefore, it is an object of the present invention to disclose an endovascular cannula that makes treatment of diseases easier, especially treatment of lung diseases. Especially, the cannula shall be appropriate for minimal invasive treatment and/or for simple surgery methods. Furthermore, a corresponding cannula system, kit and method shall be disclosed.

These objects are solved by the cannula of claim 1, by the cannula system, by the kit and by the method claim according to the independent claims. Preferred embodiments are claimed in the sub claims.

An endovascular cannula for defining a border of a transport volume for an in-vivo fluid transport within the transport volume may comprise:

a lumen portion that extends between a proximal end of the cannula and a distal end of the cannula, the lumen portion defining an inner lumen, preferably a flexible lumen portion, for instance flexible into a radial direction or into all radial directions, and

an expandable arrangement that has a non-expanded state and an expanded state.

The expandable arrangement may be switchable from the non-expanded state to the expanded state. In the expanded state, the expandable arrangement may be adapted to define at least one border of the transport volume. The border may be configured to separate and/or isolate the transport volume from a body fluid circuit.

Furthermore, the inner lumen may preferably be arranged and may preferably be configured to be in fluid communication with the transport volume in the expanded state of the expandable arrangement. The lumen portion may be configured to be guided through the body fluid circuit.

By using the proposed cannula it may be possible to use higher doses of treatment substances than up to now. This opens up more options for treatment and/or healing. Several treatment scenarios are possible. Some of them are described with regard to the Figures. Furthermore, only minimal invasive surgery may be performed for isolated lung perfusion or isolated perfusion of another organ. No thoracotomy may be necessary to isolate the lung completely from the blood circuit. This may avoid the health risks that result from thoracotomy.

The basic principle of an endovascular catheter/cannula therapy may be a treatment of vessels and/or by using vessels for the advancement of a catheter, for instance plastic tubes or plastic tubes that are armed with metal. An incision may be made into the skin of a patient. The incision may have a length that is less than 5 cm (centimeter), less than 3 cm or less than 1 cm. Local anesthesia may be used thereby. A cannula may be used to insert a guide wire and/or dilators to expand the incision and/or an opening within the vessel. The catheter may be inserted using the guide wire and/or an introducing member.

No thoracotomy may be necessary if cannulas or catheters are used. A cannula may be a tube that can be inserted into the body, often for the delivery or removal of fluid or for the gathering of data. A catheter may be a thin tube made from medical grade materials serving a broad range of functions. Catheters may be medical devices that can be inserted into the body to treat diseases or to perform a surgical procedure. Both terms “cannula” and “catheter” are used interchangeably in the following if not stated otherwise. No special surgery may be necessary, i.e. it may not be necessary that a very high specialized physician uses the proposed cannula and/or performs the proposed methods.

By modifying the material or adjusting the way cannulas/catheters are manufactured, it is possible to tailor them for cardiovascular, urological, gastrointestinal, neurovascular, and ophthalmic applications. A catheter/cannula left inside the body, either temporarily or permanently, may be referred to as an “indwelling catheter/cannula” (for example, a peripherally inserted central catheter/cannula).

Catheters and cannulas may be inserted into a body cavity, duct, or vessel. Functionally, they allow delivery and/or drainage, administration of fluids or gases, access by surgical instruments, and/or also perform a wide variety of other tasks depending on the type of catheter or cannula. The process of inserting a catheter is “catheterization”. The process of inserting a cannula is “cannulization”. In most uses, a catheter/cannula is a thin, flexible tube (“soft”) though catheters/cannulas are available in varying levels of stiffness depending on the application.

The expandable arrangement may be mounted to or connected to the lumen portion, e.g. at the distal end of cannula or at a distal part of the cannula, especially using an appropriate connection technique.

The transport volume may be fluidically, i.e. in a fluidic manner, separate and/or isolated from a natural body fluid and/or body fluid circuit, for instance from the natural blood circuit. This may result in low or no systemic leakage. The leakage rate may be equal to or smaller than 1 percent of the volume that is delivered by the fluid flow.

The transport volume may extend through at least one tissue region of an organ and/or through the organ of a subject, especially through tissue of the lung. The pressure of the fluid flow may press the fluid flow through the tissue, for instance through the tissue of the alveoli. The pressure may be in the range of 10 mm Hg (column of mercury, quicksilver) to 50 mm Hg, preferably within the range of 10 mm Hg to 30 mm Hg in order to prevent damage to the lung. However smaller or higher pressures may also be used.

The expandable arrangement may close a gap or space, preferably a circumferential gap or space, between at least one distal part of the lumen portion and at least one surrounding conduit of the body fluid circuit, e.g. vessel or chamber of the body, for instance a chamber of the heart. Thus, the isolation of the transport volume from the body fluid circuitry is reached by simple means. Clamping may not be necessary, i.e. the tissue of the vessel may not be stressed too much.

The expandable arrangement may form a tight barrier. At least 95, 96, 97, 98 or 100 percent of volume or all of the fluid flow that flows into the transport volume may be drained out of the body using the cannula or another cannula.

In the expanded state, the at least one expandable arrangement may encompass or define a volume that is greater than the volume that is encompassed by the at least one expandable arrangement in the non-expanded state, preferably by factor 2, 3 or 4 or by at least factor 2, 3 or 4. The factor may be smaller than or equal to 100, 50 or 10.

The fluid flow may flow through at least a part of the vascular system of the lung or of another organ of a subject before or after it reaches the tissue of the lung or of the organ, for instance tissue of the alveoli.

The fluid flow may consist of or contain a treatment substance that has a therapeutic effect. However, the substance may also have a toxic effect. Within the transport volume the advantages of the therapeutic effect may be greater than the disadvantages of the toxic effect. However, within the body fluid circuit the toxic effect of the treatment substance may be greater than therapeutic effect of the treatment substance. Leakage of treatment substance may be prevented by the border that is formed by the expandable arrangement. Detrimental effects or even lethal effects are also prevented in this way.

In this application document the definition for “distal” is far from a person that inserts the cannula/catheter. “Proximal” means near to the person that inserts the cannula/catheter. In the following the longitudinal axis of lumen portion or the extension thereof beyond the lumen portion may be used as a reference axis. The terms “radial”, “axial” and/or “angularly” may be used with regard to this reference axis. This is similar to the usage of cylinder coordinates that are used in a cylindrical coordinate system.

The proposed cannula may have several technical and/or medical effects:

fixation of the cannula within the body/organ/part of organ, and/or

isolation of one end of the transport volume from the body fluid circuitry,

delivery and/or drainage of the fluid flow through the cannula, especially a fluid flow that consists of or that comprises a treatment substance, and/or

positioning of an outlet or inlet of the cannula by the expandable arrangement, this may prevent the “sand blasting” effect for fluid delivery and/or that a surrounding vessel is sucked into a hole of the cannula for a drainage cannula, and/or

atraumatic endovascular insertion of the cannula because the expandable arrangement is in the non-expanded state during insertion.

The lumen portion may comprise two separate distal parts, e.g. a first distal part and a second distal part, that extend away from a bifurcation region or branching region of the lumen portion in the distal direction, e.g. by at least 1 cm, at least 1.5 cm or at least 2 cm respectively. Both distal parts may have the same length. Alternatively, distal parts having different length if compared with each other may be used. The expandable arrangement that is mentioned above may be a first expandable arrangement. The first expandable arrangement may be associated with the first distal part. The cannula may comprise a second expandable arrangement that may be associated with the second distal part.

The second expandable arrangement may have the same features and therefore the same functions and/or effects as the first expandable arrangement, i.e. it can be switched from the non-expanded state to the expanded state. In the expanded state the expandable arrangement may be adapted to define at least one border of the transport volume, and/or the border may be configured to separate the transport volume from the body fluid circuit.

The cannula may be a split tip cannula that comprises two separate expandable arrangements and that may be appropriate to be inserted into two vessels at the same time, i.e. simultaneously. Furthermore, tight borders to isolate the transport volume may be realized by the two expandable arrangements that are arranged in the two separate vessels. The vessels may be separate vessels, for instance the two left pulmonary veins or the two right pulmonary veins. Alternatively, the vessels may be branches of a main vessel that also has a bifurcation region or of vessels that are not connected to a bifurcation region. Introduction of the split tip cannula is also possible from the side of the main vessel.

The proposed split tip cannula comprising at least two split tips and at least two expandable arrangements may be ideal for isolation of the lung or of parts of the lung by endovascular insertion of the cannula, i.e. without extensive surgery. However, other organs or the brain may be treated as well using the disclosed cannula, i.e. parts that are connected to at least one body fluid circuit. The body fluid circuit may be for instance the blood circuit that comprises veins and arteries that are connected with the organ. Alternatively, the cannula may be used endovascular for the treatment of other organs, for instance of the stomach and/or of the liver, of the brain, of the kidney or of organs of the digestive system, preferably colon, of the gall bladder, of the urinary bladder, of the pancreas, etc. Furthermore, the heart may be treated, for instance using the coronal vessels to insert the cannula or the cannulas that define the isolated transport volume.

It may be contemplated to have a cannula with more than two tips and corresponding expandable arrangements. There may be for instance three tips, four tips, etc. The number of the expandable arrangements may be equal to the number of distal tips of the cannula. However, there may be at least one distal tip without an expandable arrangement.

The at least one expandable arrangement may be inflatable, especially using a fluid. The expandable arrangement may comprise a balloon that is inflatable using a liquid fluid or a gaseous fluid. The balloon may have a length of at least 2 mm (millimeter), at least 5 mm, at least 10 mm, at least 15 mm or of at least 20 mm The balloon may be shorter than 5 cm (centimeter) or shorter than 3 cm, preferably measured along the axial direction or axis of the cannula. A balloon may allow a large expansion of its volume from the non-expanded state to the expanded state. Furthermore, the outside of a balloon may be very flexible and may seal even small cavities on the inside of vessel. This may result in no or almost no leakage at the border between the isolated transport volume and the body fluid or the body fluid circuit.

The cannula may comprise at least one further lumen for guiding a fluid into and/or out of the balloon. The further lumen may be arranged at an outer surface of the cannula and may extend from the proximal end of the cannula to the balloon. The inner lumen of the cannula may not be reduced by the further volume in this case. An arrangement of the further lumen portion inside of the cannula, for instance inside the lumen portion and/or inside the inner lumen of the lumen portion may also be contemplated or realized in order to facilitate insertion of the cannula. Thus, the further lumen may be separated from the inner lumen.

The balloon may be or comprise a thin sheet of material, having for instance a thickness of less than 0.5 millimeters. Polytetrafluoroethylene (PTFE) may be an appropriate material for the material of the balloon, preferably if it is manufactured by electro spinning, for instance within an electrical field. However, other materials may also be used. The material of the balloon may form an inner sleeve and an outer sleeve that are connected to each other in a fluid tight way or that form a single piece of material.

The expandable arrangement may comprise a cage arrangement that comprises a plurality of wires, preferably at least three wires, at least four wires or at least five wires that preferably form a deformable or stretchable structure. A preferred material for the wires may be a shape memory alloy (SMA) or a shape memory material, for instance a material that changes its shape depending on the temperature of the material. Nitinol (Nickel Titanium Naval Ordnance Laboratory) is an example for such a material.

However, other materials may also be used, for instance NiTi (nickel titan), NiTiCu (nickel titan copper), CuZn (copper zinc), CuZnAl (copper zinc aluminum) and/or CuAlNi (copper aluminum nickel). Further materials that may be used are super elastic materials, stainless steel wire, cobalt-chrome alloys or cobalt-chromium-nickel-molybdenum-iron alloy. The thickness and/or diameter of the wires may be in the range of 0.1 mm (millimetre) to 2 mm, especially if only three or four wires are used within the expandable arrangement that may also be named as cage arrangement. The thickness and/or diameter of the wires may be in the range of 0.1 mm (millimeter) to 1 mm or in the range of 0.25 mm to 0.75 mm Thinner wires may be useful if more than four wires are comprised within the cage arrangement.

The expandable arrangement may comprise a membrane that may be connected to at least two of the wires of the plurality of wires. The membrane may preferably be connected to a series of at least three angularly adjacent wires of the plurality of wires but not to all wires of the plurality of wires, for instance to define an opening that faces laterally or transversally relative to a longitudinal axis of the cannula. Alternatively or additionally, the membrane may preferably be connected to all wires of the plurality of wires of the cage arrangement, for instance to define an opening that faces distally or proximally relative to a longitudinal axis of the cannula. The membrane may, in the expanded state, define an opening. The opening may, for example, face distally, laterally or proximally with regard to a longitudinal axis of the lumen portion. Additionally or alternatively, the membrane may define a volume which is fluidly connected to the lumen portion but with a greater diameter than the lumen portion, especially in the expanded state of the expandable arrangement. The membrane may be fluid tight and the opening may be an inlet into the lumen portion or an outlet out of the lumen portion.

The material of the membrane may be liquid-tight (impermeable) in both directions, i.e. from inside of diameter variable arrangement (cage) to the surrounding area and/or from surrounding area to the inside of the cage.

The membrane may be or may comprise a thin sheet of material, having for instance a thickness of less than 0.5 millimeters. Polytetrafluoroethylene (PTFE) may be an appropriate material for the membrane, preferably if it is manufactured by electro spinning, for instance within an electrical field. However, other materials may also be used. Only one sheet of membrane may be arranged for forming the membrane, e.g. there may be only one layer of membrane material. However, it is alternatively possible to have two or more layers of the membrane material. Furthermore, the membrane may be arranged distally relative to a distal end of the cannula. Insertion of the cannula may be alleviated thereby.

The flexible wires and the flexible membrane may also allow tight sealing at the border between the transport volume and the body fluid/body fluid circuit. However, alternatively, the membrane may function as a valve for the body fluid, i.e. the border is only unidirectional. This may be advantageous, for instance in order to release pressure that builds up because the transport volume is used, for instance blood pressure or afterload within the right ventricle of the heart if the transport volume is located in the main pulmonary artery, in the left pulmonary artery or in the right pulmonary artery. The valve function of the membrane may be adjusted by adjusting how tight or how loose the membrane is connected to the expandable arrangement.

The lumen portion may be adapted to guide an introducer member that preferably comprises or consists of an elongated structure, e.g. a long rod. In the case of a split tip cannula a split tip introducer member may be used or at least two separate introducer members. The expandable arrangement may comprise a contact area that is adapted to have mechanical contact with the introducer member. In the expanded state, preferably also in the non-expanded state, the contact area may overlap with an opening of the lumen portion as seen in top view onto the opening along a longitudinal axis defined by the lumen portion. The contact area may be opposite to an opening of the lumen portion. The expandable arrangement may be configured such that it changes from the non-expanded state to the expanded state if the introducer member is moved away from the contact area. The expandable arrangement may be configured such that it changes from the expanded state to the non-expanded state if the introducer member makes contact to the contact area.

“Long” may refer to a structure that has a length that is preferably equal to or at least twice the maximum width or equal to or at least the triple of the maximum width of this structure. The usage of an introducer member may ease the introduction of the cannula. The introducer member may have the further function of stretching the cage arrangement into the non-expanded state. Furthermore, the usage of the introducer member may not require an extra volume of the cannula, especially in cases in which the cannula is designed to guide fluid, especially using high flow rates of for instance 1 liter per minute or more than 1 liter per minute.

The cannula may have an insertable length that is more than 25 cm or more than 30 cm. If both jugular veins are used the cannula for the left internal jugular vein may have an outer diameter of 40 F or less. The cannula for the left internal jugular vein that may extend to the left atrium may have an insertable length of at least 45 cm or of at least 65 cm and a maximal outer diameter of for instance 31 F or less. The cannula for the left internal jugular vein that may extend to the pulmonary artery may have an insertable length of at least 75 cm and/or a maximal outer diameter of for instance 31 F or less. The outer diameter of the cannula for left pulmonary artery or for right pulmonary artery may have an outer diameter of 16 F or less.

If both jugular veins are used the cannula for the right internal jugular vein may have an outer diameter of 40 F (French, 1 French about 0.33 mm) or less. The cannula for the right internal jugular vein that may extend to the left atrium may have an insertable length of at least 25 cm or of at least 30 cm. The cannula for the right internal jugular vein that may extend to the pulmonary artery may have an insertable length of at least 55 cm or of at least 75 cm.

Both cannulas that are used to define the isolated transport volume may be inserted into the right internal jugular vein, for instance if the left internal jugular vein cannot be punctured. This may result in a very short circuitry that may allow high flow rates. Alternatively, both cannulas that are used to define the isolated transport volume may be inserted into the left internal jugular vein, for instance if the right internal jugular vein cannot be punctured. In both cases the outer diameter of the sum of both cannulas may be 35 F (French) or less. Each cannula may have an outer diameter in the range of 15 F to 18 F.

Femoral access may also be used. The femoral cannula may have an insertable length of at least 60 cm, at least 70 cm or at least 80 cm.

The insertable length of all cannulas mentioned above may be less than 1 m (meter).

The cage arrangement may add an outer diameter of 1 F to the outer diameter of the cannula in the non-expanded state and about 2 or 3 French to the outer diameter of the cannula in the expanded state.

The maximal width and/or diameter of an opening (for instance end-hole) at the distal part of the lumen portion may be less than 15 mm (millimeter), 14 mm, 13 mm, 12 mm, 11 mm, 10 mm, 9 mm, 8 mm, 7 mm, 6 mm or less than 5 mm Alternatively or additionally, the maximal width and/or diameter of an opening at the distal part of the lumen portion may be more than 1 mm, 2 mm, 3 mm or more than 4 mm or more than 5 mm, 6 mm, 7 mm or 8 mm

The at least one expandable arrangement may have a maximum diameter or a maximum width in the expanded state that allows fixation of the at least one expandable arrangement and/or placement within the main pulmonary artery, within the left pulmonary artery, within the right pulmonary artery, within a left pulmonary vein and/or within a right pulmonary vein of a subject, preferably of a subject having a body height of at least 1.5 meters or of at least 1.7 m. Thus, the subject may be an adult man or an adult woman, e.g. a body height of the subject may be more than 150 cm. However, for children or persons with less body height shorter and/or thinner cannulas may be used as well. The cannula that has one of these dimensions or even smaller dimensions may be introduced into a secondary artery of the lung or into at least one pulmonary vein.

The insertable length may refer to the part of the cannula that may be inserted into a vessel of the blood circuit. Thicker holding parts may not be inserted within the body and do not count as insertable length.

The at least one distal part of the lumen portion may comprise only one distal hole, preferably only one end-hole, preferably an end-hole that is arranged on the longitudinal axis of the at least one distal part of the lumen portion. The end-hole may preferably be arranged centrally with respect to a longitudinal axis of the cannula. The lumen portion that comprises only one distal end-hole may extend into a cage arrangement by 5 mm or by less than 5 mm Alternatively, the lumen portion may not extend into the expandable arrangement, especially not into the cage arrangement. However, the cannula may extend into the expandable arrangement further than 5 mm (millimeter) or further than 10 mm A high fluid flow may be reached if a single inlet or outlet is used for the cannula.

However, several lateral openings may be used instead or in addition to an end-hole. Lateral openings may prevent or mitigate clogging and/or a blockade of the inlet function of the cannula and/or “sand blasting” effects at outlet openings of the cannula.

The cannula may comprise at least one pre-formed bend or kink, preferably a bend that defines an angle within the range of 90 degrees to 170 degrees. The bend may ease endovascular insertion of the cannula, for instance into the lung of a body of a subject, especially jugularly through superior vena cava. The angle may be defined between two substantially straight portions of the cannula. The cannula may nevertheless be radially flexible at the bend. Thus, the cannula may be bended less or more than the angle that is pre-bended. Pre-bending may ease the insertion of the cannula and/or the force that is applied by the cannula to a vessel or a chamber of an organ, for instance to the right ventricle of the heart. The cannula comprising or having the bend may be used for endovascular jugular insertion into the left atrium or into the pulmonary artery.

The invention relates in a second aspect to a cannula system and/or cannula set for defining a border of a transport volume for an in-vivo fluid transport, especially for an in-vivo isolated fluid transport, e.g. through tissue of an organ, comprising:

a cannula according to one of the embodiments mentioned above, whereby the cannula is a first cannula, wherein the lumen portion is a first lumen portion and wherein the expandable arrangement is a first expandable arrangement, and at least one of:

a) a second cannula, that forms a first dual lumen cannula together with the first cannula and that may preferably be used to drain blood from the right half of the heart, and/or,

b) a further cannula, for instance a second dual lumen cannula or a single lumen cannula that may be used separately of the other cannulas, i.e. not as part of a dual lumen cannula, and wherein the further cannula may preferably be used as a drainage cannula for the fluid flow that comes out of the transport volume.

The second cannula may comprise a second lumen portion that extends between a proximal end of the second cannula and a distal end of the second cannula, the second lumen portion may define a second inner lumen. A part of the first cannula may be arranged or may be configured to be arranged within the second lumen portion. The distal end of the first cannula may be arranged or may be configured to be arranged outside of the second lumen portion. Thus, the first cannula and the second cannula may form a dual lumen cannula system or a multi lumen cannula system having more than two lumina for fluid transport, for instance three lumina or more than three lumina. The dual or multi lumen cannula may be a fixed cannula that does not allow relative axial movement of its cannulas with regard to each other during use, e.g. during insertion into a body of a subject.

Alternatively, the first lumen portion may be insertable into the second lumen portion such that the distal end of the first cannula is arranged outside of the second lumen portion in order to form a non-fixed dual or multi lumen cannula system with cannulas that are axially movable relative to each other, especially during use, i.e. during insertion of the cannulas into a body of a subject and/or during removal of the cannulas. The stiffness of only one cannula may be lower than the stiffness of the cannula system. This may allow less traumatic endovascular insertion of the cannulas and/or removal, i.e. the outer cannula first with reduced stiffness and then the inner cannula inside of the outer cannula without contact and/or without friction to vessels in which the outer cannula is already arranged. The cannulas may be introduced and/or removed in sequence relative to each other.

The expandable arrangement of the first cannula may be a first expandable arrangement. A second expandable arrangement may be arranged or mounted or otherwise associated with the second lumen portion, preferably on a distal part of the second lumen portion. The second expandable arrangement may have a non-expanded state and an expanded state. The second expandable arrangement may encompass or define in the expanded state a volume that is greater than the volume that is encompassed or defined by the second expandable arrangement in the non-expanded state. The expansion of the second expandable arrangement may be based on a different principle of physics compared to the principle of physics on which the expansion of the first expandable arrangement is based, i.e. balloon versus cage. The second expandable arrangement may be second fixation means and/or may also fulfill other and/or additional functions.

The second expandable arrangement may comprise a cage arrangement that comprises a plurality of wires, preferably at least three wires, at least four wires or at least five wires. The second expandable arrangement may comprise no membrane. This may be advantageous if the cage arrangement is arranged with the right atrium of the heart or within the right ventricle because blood may reach the distal end of the second cannula, i.e. there is no blockade by a membrane or other structure, for instance a balloon. Drainage from all sides may be possible with a chamber of the heart that is in steady pumping movement. At least one wire of the cage arrangement may be omitted, e.g. there may be a larger gap between two of the wires of the cage arrangement compared to the gap or gaps between other wires that are adjacent to each other.

Alternatively, a membrane may be connected to at least two of the wires of the plurality of wires, to a series of adjacent wires of the plurality of wires but not to all wires of the plurality of wires or to all wires of the plurality of wires of the second cage arrangement. The technical effects of these membrane features may be similar to the technical and/or medical effects that are mentioned above for the first expandable arrangement/cage arrangements. The membrane may comprise an opening that faces distally with regard to a longitudinal axis of the second lumen portion or that faces transversally with regard to a longitudinal axis of the second lumen portion. Alternatively, the second expandable arrangement may be inflatable and/or may comprise a balloon. Both alternatives, i.e. membrane and/or balloon, may open up new applications in medicine.

The second cannula may comprise a first group of holes and a second group of holes. The smallest distance between any arbitrarily selected pair of holes having one hole in the first group and the other hole in the second group may be more than the minimum distance between any arbitrarily selected pair of holes having both holes within the first group of holes or having both holes within the second group of holes, preferably more than twice the minimum distance. The first group of holes may be arranged more distally than the second group, e.g. such that the first group is adapted to be placed within the right ventricle and/or the second group is adapted to be placed within the right atrium of the heart of the subject if the cannula is inserted for instance jugularly. Thus, there may be an intermediate portion of the second cannula between the two groups of holes. Within this intermediate portion the cannula may be laterally closed, i.e. hole-free. The portion that is laterally closed may have a length, preferably an axial length, of at least 10 mm (millimeter), 15 mm, 20 mm or of at least 25 mm The portion that is laterally closed may have a length, preferably an axial length that is less than 10 cm (centimeter) or less than 7 cm.

The holes within the groups may be lateral holes. Right heart assist may be done almost completely if two or more groups of holes are used for drainage within the right half of the heart, i.e. within right atrium and/or right ventricle. A third group of holes may be arranged in the vena cava, especially within the superior vena cava. Afterload that might occur to the heart if the main pulmonary artery, the left pulmonary artery or the right pulmonary artery is closed may be reduced significantly.

The cannula system may comprise a first introducer member that may be adapted to be introduced within the first cannula. If a cage arrangement is used, the first expandable arrangement may be in the non-expanded state if the first introducer member is in contact with a contact portion of the first expandable arrangement. The first expandable arrangement may be in the expanded state if the first introducer member is not inserted within the first lumen portion. Thus, the introducer may be used for several purposes as described above.

The cannula system may form a single entity that is sold and delivered in a separate package. The package may include single items that are delivered within bags that are sealed, preferably plastic bags. The items within the bags may be sterilized before packaging. The entity may have an authorization from a state agency that is responsible for the security and/or compliance of medical devices, for instance the Food and Drug Agency (FDA) in the United States of America (USA) or a similar agency of another country.

Furthermore, the cannula system may comprise a second introducer member that is adapted to be introduced within the second cannula and that is different from the first introducer member, preferably shorter and/thicker than the first introducer member. Thus, both introducer members may be adapted to the respective cannula to ease the introduction as much as possible and to reduce the danger of injuries for the patient. Further technical and/or medical effects of an introducer member are mentioned above and may apply to the second introducer member as well.

Alternatively and/or additionally to the second cannula the cannula system/set may comprise the further cannula. The further cannula may be configured to be used independently of the first cannula. The further cannula may comprise a further lumen portion that extends from a proximal part of the further cannula to at least one distal part of the further cannula. The further lumen portion may define a further inner lumen. There may be at least one further expandable arrangement associated with the at least one distal part of the further lumen portion, e.g. mounted or connected to the at least one distal portion of the further cannula. The further lumen portion may be flexible, preferably radially flexible relative to a longitudinal axis of the further cannula.

The cannula system or cannula set may comprise:

a single lumen first cannula that may be adapted to define at least one first border on one side of the transport volume,

and the further cannula that may be adapted to define at least one second border on the other side of the transport volume.

The cannula system/set may form one single entity that is sold and delivered in a separate package. The package may include single items that are delivered within bags that are sealed, preferably plastic bags. The items within the bags may be sterilized before packaging. The entity may have an authorization from a state agency responsible for the security and/or compliance of medical devices, for instance the Food and Drug Agency (FDA) in the United States of America (USA) or a similar agency of another country.

Alternatively, the cannula system or cannula set may comprise:

a first cannula of a dual lumen cannula, wherein the first cannula may be adapted to define at least one first border on one side of the transport volume,

a second cannula of the dual lumen cannula, wherein the second cannula may be adapted to be used for drainage of blood from the right side of the heart, i.e. for right heart support or assist,

and the further cannula that may be adapted to define at least one second border on the other side of the transport volume.

This cannula system/set may also form one single entity that is sold and delivered in a separate package. The package may include single items that are delivered within bags that are sealed, preferably plastic bags. The items within the bags may be sterilized before packaging. The entity may have an authorization from a state agency that is responsible for the security and/or compliance of medical devices, for instance the Food and Drug Agency (FDA) in the United States of America (USA) or a similar agency of another country.

For both systems/sets, the first cannula may be a delivery cannula for the fluid flow through the transport volume that is located at least partially within an organ, for instance within the lung, and the further cannula may be a drainage cannula for this fluid flow. The flow may be an antegrade flow through the organ, especially through the lung, i.e. in the natural direction. The fluid flow may be directed especially through at least two vessel of the organ. Alternatively, the flow may be a retrograde flow.

For both systems/sets, it is alternatively possible, that the first cannula is a drainage cannula for the fluid flow through the transport volume/an organ, for instance through the lung, and that the further cannula is a delivery cannula for this fluid flow. Thus, it is possible to drive an antegrade or a retrograde flow through the transport volume that extends through tissue of an organ, especially through the lung. The fluid flow may be directed especially through at least two vessels of the organ. In the case of a retrograde flow through the lung, side branches of vessels between the pulmonary and bronchial venous system may be exploited to deliver drugs to the tissue of the lung, for instance to metastatic lesions.

The further cannula may have an insertable length that is more than 15 cm, 20 cm or more than 25 cm, but less than 1 m (meter). If the further cannula is not a split tip cannula the length that are given above for the first cannula may also be valid for the further cannula.

A maximal width or diameter of an opening at the distal part of the lumen portion may be less than is less than 15 mm, 14 mm, 13 mm, 12 mm, 11 mm, 10 mm, 9 mm, 8 mm, 7 mm, 6 mm, or less than 5 mm This may make the further cannula appropriate for insertion into vessels of the lung, for instance into at least one pulmonary vein. These values are valid if the further cannula is not a split tip cannula. However, if the further cannula is a split tip cannula the widths or diameter may be divided by two.

A maximal width or diameter of an opening at the distal part of the lumen portion may be more than 5 mm, 6 mm, 7 mm or 8 mm These values may be valid if the further cannula is not a split tip cannula. However, if the further cannula is a split tip cannula the widths or diameter may be divided by two.

There may be only one hole at a distal part of the further cannula, for instance only one end-hole. The width or diameter should allow to deliver a sufficient amount or volume per minute of the fluid flow that comprises preferably a treatment substance. The flow rate may be the same as in the case of normal lung function. Alternatively, the flow rates may be below or above the value of the natural flow rate, for instance if the body of a subject rests.

The at least one further expandable arrangement may have a maximum diameter or width in the expanded state that allows fixation of the further expandable arrangement and/or placement within the main pulmonary artery, within the left pulmonary artery, within the right pulmonary artery, within a left pulmonary vein and/or within a right pulmonary vein of the subject. Thus, the further cannula may be appropriate for treatment of the lung.

The further lumen portion may comprise two separate distal parts that extend away from a bifurcation region of the further lumen portion in the distal direction, preferably by at least 1 cm, at least 1.5 cm or at least 2 cm respectively. Both distal parts may have the same length or may have different length if compared with each other. In one embodiment the first cannula may not be a split tip cannula and the further cannula may be a split tip cannula.

Moreover, a first distal part of the further lumen portion may be associated with a first further expandable arrangement of the at least one further expandable arrangements distally from the bifurcation region. A second distal part of the lumen portion may be associated with a second further expandable arrangement of the at least one further expandable arrangement distal from the bifurcation region. The association may be realized by mounting and/or connection techniques.

Therefore, the further cannula may be a split tip cannula that comprises at least two or at least three distal tips. Each distal tip may be associated with a respective expandable arrangement. This makes the further cannula appropriate for new medical applications, for instance for insertion into both left pulmonary veins or into both right pulmonary veins, especially endovascular and/or transseptal, preferably through the atrial septum of the heart or through the ventricle septum of the heart. This feature may be a key feature for a broad range of medical applications for treatment of the lung or of other organs.

The cannula system or set of cannulas may comprise an inner cannula that is preferably insertable and/or that is configured to be arranged within the further cannula and that is adapted for the delivery of oxygenated blood into an artery of the subject during the in-vivo fluid transport through the transport volume, that may extend at least partially within the tissue of an organ, preferably of the lung of the subject. Thus, the further cannula may be part of a dual lumen cannula system, for instance of a second dual cannula system of the cannula system or cannula set. If two dual lumen cannula systems are included in the cannula system/set, there may be only two introducer members. Alternatively, there may be three or four different introducer members, each having a different length and/or different width/diameter with regard to the other introducer members that are different from each other, e.g. in length and/or in maximum diameter. The dual or multi lumen cannula system that comprises the further cannula may be a fixed one or a non-fixed one, see description above for details. The outer cannula of a dual or multi lumen system that comprises the further cannula may also have expandable arrangement, for instance for placement within the left atrium. Introduction of the further cannula into pulmonary veins may be eased using the expandable arrangement that is located within the left atrium.

The cannula system or set of cannulas may alternatively comprise a single lumen cannula that is adapted for the delivery of oxygenated blood into an artery of the subject, preferably femoral and/or transcaval during the in-vivo fluid transport through the transport volume that is arranged within an organ, preferably of the lung of the subject. The single lumen cannula may be used separately and/or independently of the further cannula and of the other cannulas of the cannula system/set.

A third aspect of the invention relates to a kit for defining at least one border or at least two borders of a transport volume for an in-vivo fluid transport, comprising:

a cannula according to one of the embodiments mentioned above, and/or

a cannula system/set according to one of the embodiments mentioned above, and:

at least one pump for driving a fluid flow through the transport volume, preferably a roller pump (pulsatile flow) or a membrane pump (pulsatile flow) or a centrifugal pump (pulsatile flow or continuous flow or switchable between both of these operation modes) or a diagonal pump (pulsatile flow or continuous flow or switchable between both of these operation modes) or an axial pump (pulsatile flow or continuous flow or switchable between both of these operation modes), and/or

a delivery unit for the introduction of at least one treatment substance into the fluid flow in order to guide at least one treatment substance into the transport volume via the fluid flow, and

preferably at least one removing unit for filtering out the at least one treatment substance and/or other particles from the fluid flow, e.g. a filter unit.

The kit may form one single entity that is sold and delivered in a separate package. The package may include single items that are delivered within bags that are sealed, preferably plastic bags. The items within the bags may be sterilized before packaging. The entity may have an authorization from a state agency that is responsible for the security and/or compliance of medical devices, for instance the Food and Drug Agency (FDA) in the United States of America (USA) or a similar agency of another country.

The fluid flow may be transported into the transport volume, through the transport volume and then out of the transport volume using the parts or items of the kit, preferably using a closed hydraulic circuitry. The delivery unit may allow dosage of drugs or treatment substances and/or mixing of the at least one drug/treatment substance with a carrier fluid of the fluid flow.

The at least one treatment substance may be a substance for treating cancer or an infectious disease, preferably lung cancer or pneumonia. The treatment substance may be delivered separately from the kit — this may ease certification of the kit—or may be part of the kit, i.e. logistics may be simplified for the user of the kit. Thus it is possible to sell two kinds of different kits, i.e. one with the treatment substance and one without the treatment substance. The treatment substance may be one of the substances that are mentioned below.

With regard to the pump or pumps that may be comprised within the kit the following may apply:

wherein only one pump is comprised within the kit and wherein the pump is adapted to create a flow through the transport volume within a range of 0.5 liter per minute to 1.0 liter per minute, i.e. greater or equal than 0.5 liters and/or equal or less than 1.5 liters, wherein the pump is preferably a roller pump, or

wherein only one pump is comprised within the kit and wherein the pump is adapted to create a flow through the transport volume within a range of 1.5 liters per minute to 3.5 liters per minute, i.e. greater or equal than 1.5 liters and/or equal or less than 3.5 liters, wherein the pump is preferably a centrifugal pump, or

wherein only one pump is comprised within the kit and wherein the pump is adapted to create an overall flow through the transport volume and through a further part of the body of a subject, preferably comprising oxygenated blood, within a range of 3.5 liters per minute to 5.0 liters per minute, i.e. greater or equal than 3.5 liters and/or equal or less than 5 liters, wherein the pump is preferably a centrifugal pump, or

wherein at least two pumps are comprised within the kit and wherein the pumps are adapted to create an overall flow through the transport volume and through a further part of the body of a subject, preferably comprising oxygenated blood, within a range of 5 liters per minute to 8 liters per minute i.e. greater or equal than 5 liters and/or equal or less than 8 liters, wherein the pumps are preferably centrifugal pumps.

Thus, the pump is adapted or the pumps are adapted to the amount of fluid flow that is appropriate for treatment, especially for treatment of the lung or of another organ.

Moreover, the kit may comprise at least one, at least two, at least three, at least four arbitrarily selected or all of the following units or elements:

an oxygenator unit for oxygenating blood and/or the fluid flow, and/or

a carbon dioxide removal unit for removing carbon dioxide from blood and/or the fluid flow, and/or

an inhalation unit that enables the inhalation of a treatment substance by the subject, wherein the inhalation unit is configured to enable inhalation of the treatment substance while, preferably only while, driving the fluid flow through the transport volume, and/or

at least one Y-connector, e.g. for connecting the cannula to at least one other unit or element of the kit, and/or,

at least one unit for controlling a respective pump of the kit in pulsatile pumping mode or in continuous flow mode or that is adapted to switch between pulsatile mode and continuous mode.

The kit may comprise for instance two single lumen cannulas and a pump/control unit that is switchable between pulsatile mode and continuous mode thereby opening up new medical application scenarios.

These parts or items may allow the enforcement of at least one of the treatment methods that are mentioned below or of other medical treatment methods. Inhalation during the perfusion of the transport volume in the lung may allow the treatment of the lung alveoli from two sides at the same time, e.g. from the outside and from the inside. This may improve therapeutic effects, i.e. there may be synergistic effects between two administration forms of treatment substances/drugs.

The treatment substance that is applied by inhalation may be a substance for treating cancer or an infectious disease, preferably lung cancer or pneumonia. Alternatively and/or additionally the treatment substance that is applied by inhalation may promote the therapeutic effect of the treatment substance that is introduced into the fluid flow and that is transported through the transport volume.

A fourth aspect of the invention relates to a method for an in-vivo fluid transport, especially for an isolated in-vivo fluid transport, within a transport volume, preferably within a fluidically isolated transport volume, comprising:

inserting a cannula endovascularly into a body fluid circuit or into a body fluid system,

wherein the cannula comprises a lumen portion that extends between a proximal end of the cannula and a distal end of the cannula, the lumen portion defining preferably an inner lumen,

forming at least one border within the body fluid circuit or body fluid system to delimit the transport volume from the body fluid circuit or body fluid system,

wherein the border is arranged within a conduit of the body fluid circuit or of the body fluid system and

wherein the border blocks a body fluid flow within the conduit of the body fluid circuit or of the body fluid system along the cannula beyond the border, and

after forming the border, guiding a fluid flow through the cannula into the transport volume wherein the at least one border is arranged to separate and/or to isolate the transport volume from the body fluid circuit or body fluid system.

The body fluid circuit/system may be closed or open, i.e. it may form a body fluid system. An example for a closed body fluid system is the blood circuit system.

The isolation may refer to a fluidic separation. The border may be realized by at least one border element. The at least one border element may be mounted on, preferably connected to, the cannula or may be more loosely associated with the cannula.

The transport volume may be delimited by:

the at least one border, and/or

by vessels that lead to an organ, and/or

by tissue of the organ, preferably by tissue that is not part of the transport volume, and/or

by parts of the cannula.

The transport volume may be sealed from the body circuit, i.e. there may be no leakage or no significant leakage. This sealing may allow the treatment of the organ with toxic substances for other organs and/or with high concentration of substances that would be toxic for other organs of a body of a patient or subject. An endovascular treatment of a wide range of organs is feasible, for instance lung, kidney, liver, stomach, stomach combined with liver, gall bladder, urinary bladder, other organs of digestive system, brain, heart, etc.

The duration for flowing the fluid flow through the transport volume may be less than one hour, or less than 30 minutes, or less than 15 minutes. The duration for flowing the fluid flow through the transport volume may be more than 5 minutes or more than 10 minutes. Temperature, flow rate, pressure and concentration of treatment substances may be adapted to the specific needs of the patient or subject and with regard to the medical treatment that is applied.

At least a part of the transport volume may be arranged within the lung of a subject or within a single lobe of lung (L) or within a part of a single lobe of lung (for instance the upper part or the lower part), preferably within tissue of the lung or preferably within tissue of the alveoli of the lung. The lung is appropriate to create an isolated transport volume because it receives its arterial blood supply almost exclusively from the pulmonary artery and drains only into four pulmonary veins. If only one half of the lung, e.g. one lobe of the lung is treated, there are only three passages that have to be closed in order to reach a complete isolation. The same may be true for other organs or part of these organs.

The transport volume may extend only within one lobe of the lung of a subject or only within a part of one lobe of the lung. This may allow selective treatment of the lung. Thus a disease may only extend to one lobe or an embolism/thrombus may be located only in one lobe. However, the treatment of only one lobe at a time may also be advantageous if the disease extends to both lobes of the lung, especially to the whole lung. It may be advantageous to treat only a part of the lung/organ in order to mitigate negative effects of the treatment, especially the weakening of the organ. Furthermore, the natural function of the untreated part of the organ may be used during the treatment to keep the patient alive. This may allow to reduce the effort to assist or support the respective organ or of other organs that are functionally connected to the respective organ. The lung is for instance functionally related to the heart.

Thus, right heart assist and/or lung assist may not be performed during guiding the fluid flow through the transport volume, i.e. during the treatment process, especially if only a part of the lung/organ is treated. However, even if only a part of the lung/organ is treated right heart assist and/or lung assist may be performed, for instance in order to support the overall body function, especially the main body functions, blood transport and/or oxygen transport and/or carbon dioxide removal.

The body fluid circuit may be the blood circuit and at least 25 percent, at least 50 percent, at least 75 percent or 100 percent of the blood flow rate that flows during treatment through the vena cava into the heart, preferably into the right atrium and/or into the right ventricle, may be pumped by a heart of a subject into the part of the lung/organ that does not comprise the transport volume. A part of the blood fluid circuit remains open, for instance the part in which the transport volume does not extend although another part of the blood circuit is blocked by the isolated transport volume. This means that no heart support may be necessary or only slight heart support.

At least 25 percent, at least 50 percent, at least 75 percent or 100 percent of the volume of oxygen that is transported during treatment into the heart and/or into the body of the subject may originate from the part of the lung that does not comprise the transport volume.

Alternatively or additionally, at least 25 percent, at least 50 percent, at least 75 percent or 100 percent of the removal of carbon dioxide from the blood of the subject may be done by the part of the lung that does not comprise the transport volume. This means that no lung support may be necessary or only slight lung support.

The transport volume may be a first transport volume. During a first duration, the first transport volume may be used to guide the fluid flow, for instance through a first part of the lung. During a second duration that follows after the first duration a second transport volume may be used that is different from the first transport volume. The second transport volume may be used preferably after changing the location of the cannula and/or of another cannula.

There may be the following variants:

a) The first transport volume is a subset or a sub volume of the second transport volume or the second transport volume may be sub volume of the first transport volume. For instance, treatment of the whole lung first and then only of a lobe of the lung or vice versa. For instance, treatment of a single lobe of the lung first and then or thereafter treatment of a part of this lobe or vice versa.

b) The first transport volume may be completely different from the second transport volume, i.e. there may be no overlapping parts. For instance treatment of the left lobe first and then of the right lobe or vice versa. For instance, treatment of a part of a lobe first and then treatment of another part of this lobe or vice versa, for instance first upper lobe and the n lower lobe or vice versa.

c) The first transport volume and the second transport volume may have a common part and there may be a part in each transport volume that is not part of the other transport volume.

The time between the first duration and the second duration may be less than one hour, or less than 30 minutes, or less than 15 minutes. The time between the first duration and the second duration may be more than 5 minutes or more than 10 minutes. Furthermore, the first duration may be less than one hour, or less than 30 minutes, or less than 15 minutes. The first duration may be more than 5 minutes or more than 10 minutes. Moreover, the second duration may be less than one hour, or less than 30 minutes, or less than 15 minutes. The second duration may be more than 5 minutes or more than 10 minutes. However, other duration times are possible as well.

The first transport lumen may be arranged in a first lobe of the lung and the second transport volume may be arranged in a second lobe of the lung that is different from the first lobe of the lung. The first lobe may be the left lobe and the second lobe may be the right lobe. Alternatively, the first lobe may be the right lobe and the second lobe may be the left lobe of the lung. It may be easier to treat the left lobe if the cannula is inserted through the atrial septum and the left atrium. However, the first transport volume and the second transport volume may alternatively be arranged within the same lobe of the lung, e.g. within the right lobe or within left lobe.

The lumen portion may comprise a first distal part and a second distal part which extend away from a bifurcation region of the lumen portion into the distal direction, preferably by at least 1 cm (centimeter), at least 1.5 cm or at least 2 cm respectively. The border may be a first border that may be arranged distally from the bifurcation region and that may be associated with the first distal part. The conduit that is mentioned above may be a first conduit. The second distal part may be arranged within a second conduit of the body fluid circuit. A second border or a second border element may block a body fluid flow within the second conduit of the body fluid circuit along the second distal part beyond the second border. The second border may be arranged distally from the bifurcation region and may be associated with the second distal part. The usage of a split tip cannula comprising at least two tips and at least two border elements may have the advantages mentioned above.

Preferably, the first distal part may be arranged within a first one of the left pulmonary veins and the second distal part may be arranged within a second one of the left pulmonary veins. It may be comparably easy to introduce the split cannula from the right atrium through the atrial septum and through the left atrium into the left pulmonary veins. Treatment of part of the lung, for instance of only the left lobe is possible in this way. The bifurcation region may be arranged within the left atrium and/or within the right atrium, i.e. the atrial septum may be punctures twice, i.e. once for each distal part.

Alternatively, the first distal part may be arranged within a first one of the right pulmonary veins and the second distal part may be arranged within a second one of the right pulmonary veins. Separate treatment of the right lobe of the lung may be possible. However, the usage of a cage arrangement carrying a membrane may avoid the insertion of a split tip cannula into the right pulmonary veins and the sharp bend of the split tip cannula that is necessary. The bifurcation region may be arranged within the left atrium and/or within the right atrium.

However, the split tip cannula may also be used for insertion into the right pulmonary veins, for instance if passed through the ventricle septum and through the left ventricle and through the left atrium. The bifurcation region may be arranged within the left atrium and/or within the left ventricle in the latter case. It is also possible to puncture the ventricle septum twice, for instance if the bifurcation is located within the right ventricle or within the right atrium.

Partial treatment of the lung opens up the following variants for treatment:

a) the transport volume may be arranged in one lobe of the lung and only this lobe may be treated only one time by guiding a treatment substance into the transport volume through the cannula. The other lobe of the lung may not be treated, especially not within one year and/or not with endovascular cannula(s). This may allow a selective treatment of the lung. More selectivity is possible if only a part of a lobe of the lung is treated. The part of the lobe may comprise less than one third or less than one half of a lobe of a lung, for instance based on a footprint area of the lung using a projection to the coronal or frontal plane of the body, a volume of tissue of alveoli or on another criteria that is appropriate.

b) wherein the transport volume is arranged in one lobe of the lung and wherein only this lobe is treated several times by guiding a treatment substance into the transport volume, and wherein the other lobe of the lung (L) is not treated. The duration between two treatment sessions may be at least one month (30 days), at least one week or at least 24 hours after the treatment of the lobe of the lung (L) that is treated. A treatment session may be defined by insertion and removal of at least one cannula. The time between the treatment sessions may allow the treated part of the lung and/or the overall body of the subject to recreate.

c) wherein the transport volume may be arranged in one lobe of the lung and wherein within one treatment session only this lobe may be treated by guiding a treatment substance into the transport volume and wherein the other lobe of the lung is not treated within the same treatment session but within a different treatment session, and wherein preferably the duration between both treatment sessions is at least one month, at least one week or at least 24 hours. A treatment session may be defined by insertion and removal of at least one cannula. The time between the treatment sessions may allow the treated part of the lung and/or the overall body of the subject to recreate. Although, treatment of the whole lung is necessary only single lobes or parts of a single lobe are treated in order to allow the lung to adapt better to the stress that is involved with the treatment.

Alternatively, both lobes of the lung may be treated simultaneously, i.e. at the same time, by guiding a treatment substance into the transport volume through the cannula. Right heart support, preferably by draining blood from right atrium and/or from right ventricle, and/or lung support may be performed during guiding the fluid flow through the transport volume, i.e. during the treatment, preferably by delivering oxygen into the blood of the treated subject and/or by removal of carbon dioxide from the blood of the subject. Preferably a further cannula may be used to perform heart support and/or another cannula may be used to perform lung support. At least one dual lumen cannula may be used in order to have a less traumatic treatment.

The fluid flow may be directed antegrade and/or retrograde relative to the natural body fluid flow direction in the conduit of the body fluid circuit if the whole lung is treated or if only a part of the lung is treated. During a first treatment step, the fluid flow may be directed into a first flow direction within the transport volume and during a second treatment step that follows after or that is before the first treatment step the fluid flow within the transport volume may be directed into a second flow direction that is a reverse direction relative to the first direction.

The time between the first treatment step and the second treatment step may be less than one hour or less than 30 minutes, preferably more than 5 minutes or more than 10 minutes. The duration of one treatment step may be less than one hour or less than 30 minutes or less than 15 minutes, preferably more than 5 minutes or more than 10 minutes. However, shorter times may also be contemplated.

The change between retrograde flow and antegrade flow or vice versa may be combined with the treatment of different parts of an organ. The change of the flow directions may be especially relevant to the treatment of embolism etc., for instance to remove a blood thrombus, a fat thrombus, air, etc. The change between retrograde flow and antegrade flow or vice versa may be relevant for the treatment of the lung or of another organ.

The border may be formed by a border element that comprises an expandable arrangement. The expandable arrangement may have a non-expanded state and an expanded state. The expandable arrangement may be switched or is switched from the non-expanded state to the expanded state, preferably after it has reached its final position within a body. The volume that is encompassed or defined by the expandable arrangement in expanded state may be at least by factor two, three or four greater than the volume in the non-expanded state. The same technical and/or medical effects may apply that are mentioned above.

The expandable arrangement may comprise at least one balloon that is inflatable using a liquid fluid or a gaseous fluid, for instance Xenon. The balloon may have an axial length along a longitudinal axis of the cannula of at least 5 mm (millimeter), at least 10 mm, at least 15 mm or of at least 20 mm and is preferably shorter than 5 cm (centimeter). The balloon may be appropriate for inflation within an artery or a vein of the lung or of another organ.

The cannula may comprise a further lumen that may guide a fluid into and/or out of the balloon. The further lumen may be arranged at an outer surface of the cannula and may extend from the proximal end of the cannula to the balloon. Alternatively, the further lumen may be arranged within the inner lumen of the lumen portion. The further lumen may comprise a fluid that is used as an auxiliary fluid and that makes no direct physical contact with the body, especially with the organ that is treated. The material of the balloon, for instance a membrane material, and the further lumen may shield the auxiliary fluid from the body. Details of the material of the balloon are mentioned above.

Alternatively, the expandable arrangement may comprise a plurality of wires, preferably at least two, at least three or at least four wires, preferably wires that form a deformable, especially a stretchable, supporting structure that may for instance be deformed or stretched by an introducer member. The expandable arrangement may comprise at least one membrane that is connected to at least two of the wires of a plurality of wires. A preferred material for the wires is a metal and/or a shape memory alloy (SMA) or material, especially one of the materials mentioned above.

Preferably, the membrane may define an opening that faces laterally and wherein the membrane may be positioned such that the opening faces to at least one or to both of the right pulmonary veins. After the positioning of the membrane a blood flow from at least one or from both of the left pulmonary veins to the left ventricle may still be possible or may not be possible to at least 50 percent of the flow rate compared to the case in which no cannula is introduce within the left atrium. The usage of a membrane may be an easy way to isolate the right lobe of the lung or parts of the right lobe.

However, it may be contemplated to use a similar arrangement also for the isolation of the left lobe of the lung or of parts of the left lobe of the lung, i.e. the membrane may be arranged within the cage and may have an opening that faces distally to the pulmonary veins. The distal part of the cannula may extend into the cage, for instance more than 10 mm Blood from the right pulmonary veins may flow between the right wall of the left atrium and the membrane to the left ventricle in the latter case.

The fluid flow may be guided through the cannula from outside of the body of a subject into the body of the subject. Alternatively, the fluid flow may be guided through the cannula out of the body of the subject. Thus an extracorporeal circuit is used outside of the body. This avoids miniaturization of components and/or implantation of components of the circuit that is used for fluid transport, especially of pumps.

The fluid flow may comprise and/or consist to at least 20 percent or at least 60 percent or at least 80 percent of volume of blood or of components of blood. Blood may have the advantage that it is the natural fluid that flows through the conduit or vessel and that the transport volume within the organ may be optimized to transport blood. However, alternatively or additionally, other carrier fluids may be used as well, for instance based on a saline solution, for instance a normothermic saline solution or a hyperthermic saline solution. Voluven (may be a registered trademark), Ringer's (may be a registered trademark) solution, especially lactated Ringer's solution and/or Hespan (may be a registered trademark) may also be used, especially in combinations with each other.

Blood thinning substances may be used if blood is used as fluid flow or as the main carrier within the fluid flow, especially if heated above normothermic temperature. The blood thinning substance may prevent clogging or the blood. Heparin (may be a registered trademark) or other substances may be used. The INR-value (international normalized ratio) may be determined from the blood in order to adjust the blood coagulation or the blood clotting. Other values may be used as well.

The fluid flow may comprise at least one medicament and/or treatment substance, preferably a medicament for treating cancer and/or a treatment substance for treating cancer, preferably lung cancer, or a lung infection or for cleaning the lung. A pharmaceutical active substance may be used. Stem cells may also be used, for instance stem cells that strengthen the tissue of the organ, especially of the lung. The stem cells may promote the healing of damaged tissue directly or indirectly by strengthening of the immune system of the body or of the organ that is be treated. Alternatively or additionally, the stem cells may stimulate growth of the tissue of the organ.

The cleaning substance for cleaning the lung or another organ may be bases on a physiological neutral solution, e.g. a saline. Chalk, thrombus or other particles may be removed by the cleaning substance. A CO₂ (carbon dioxide) portion may promote the cleaning. A pulsatile cleaning flow may be used or a continuous flow.

There may be a port which is configured to receive the medicament and/or the treatment substance. The port may be part of a delivery device that is connected to the cannula or to another part of the fluid flow circuit. The treatment substance may be a chemical or biochemical substance that is added to the fluid/blood, for instance a substance for a chemotherapy. The medicament and/or the treatment substance may be used in the treatment of a disease selected from the group comprising or consisting of cancer or an infectious disease. Alternatively, the medicament and/or the treatment substance may be used to clean the organ, i.e. to remove substances and/or particles that are detrimental for the basic function of the organ, especially the lung.

A further treatment substance may be applied by inhalation in addition to the fluid flow through the transport volume, preferably simultaneously to the fluid flow, i.e. at the same time. The inhalation may take place alternatively and/or additionally before or after the treatment using the transport volume, for instance within the same day (24 hours) or within the same week as the fluid flow through the transport volume. The inhalation substance may be a substance for treating cancer, preferably lung cancer, or for treating a lung infection, especially caused by bacteria and/or virus and/or fungi, for instance pneumonia. Alternatively and/or additionally, the inhalation substance may clean the lung. The inhalation substance may be or comprise a pharmaceutical active substance. The inhalation substance may be the same substance as the substance that is used for perfusion within the transport volume. Alternatively, substances that are different from each other may be used for perfusion and inhalation.

A radiology (radioactive) treatment, e.g. using electrons and/or protons and/or neutrons and/or other ionized particles, preferably radioactive oncology or radio-oncology treatment, may be applied in addition to the fluid flow through the transport volume, preferably simultaneously, i.e. at the same time, to the fluid flow, within the same day, within the same week or within the same month as the fluid flow. The radiation may be ionizing, i.e. contain high energy particles or high energy electromagnetic waves. Radioactive radiation may be used. Cancer cells may be destroyed by the radiation.

A treatment substance may be applied using nano particles or micro particles, preferably within the fluid flow and/or within the inhalation gas and/or in order to perform the radiology treatment. The particles may be balls or beats. Liposomes or other molecules may be used to include or to encircle the treatment substances. The usage of nano particle or micro particles may promote the effect of the treatment substances.

The fluid flow may be guided out of the body of a subject using the cannula or a further cannula. The fluid flow may be extracorporeal cleaned and/or filtered using a filter unit, especially using a filter unit that comprises at least one a fluid filter and/or at least one adsorption element and/or at least one absorption element and/or at least one permeable or semipermeable membrane for filtering and/or dialyzing element (for instance based on electric separation principles and/or on pressure gradients). The filtering may have several effects:

removing the treatment substance, this may be the basis for a new adaption of the concentration, and/or

preventing that detrimental particles get to the organ again, and/or

fulfill medical standards.

Preferably, the cleaned and/or filtered fluid flow is used again to be guided through the transport volume, in order to perform the treatment with a low overall amount of fluid. Especially, blood may not be available in great amounts. The own blood of a person or a patient may be rare and therefore valuable.

The fluid flow may be guided out of the body of a subject using the cannula. The oxygen contents may be increased or decreased and/or the contents of carbon dioxide of the fluid flow may be increased or decreased extracorporeally. The fluid flow that has a changed content of oxygen and/or carbon dioxide may be used to be guided through the transport volume. Hyperoxygenation, for instance above normal values, or hypo-oxygenation, for instance below normal values, may have effects for the absorption and/or the reaction of the tissue of the organ to medicaments and/or treatment substances.

Only one pump may be used for driving the fluid flow through the transport volume and for at least one of draining out body fluid and deliver cleaned/or oxygenated body fluid into the body of a subject. Thus only one pump may be used in a simple circuitry to fulfill several functions. The pump may generate a pulsed fluid flow, i.e. the natural kind of flow—i.e. clogging of blood may be impeded by turbulences caused by the pulsation, or a continuous fluid flow that may allow higher flow rates compared to a pulsed fluid flow.

Alternatively two or more than two pumps may be used. A first pump may be used for driving the fluid flow through the transport volume. A second pump may be used for the delivery of oxygenated blood into the body and/or for drainage of body fluid out of the body of a subject. At least one of the pumps may generate a pulsed fluid flow or both of the pumps may generate a pulsed fluid flow. The pumps may be adjusted separately to the appropriate flow rate. Pulsed fluid flow is the natural kind of fluid flow through the organ that is treated.

Alternatively, at least one of the pumps may generate a continuous fluid flow or both of the pumps may generate a continuous fluid flow. Continuous flows may allow higher flow rates.

Alternatively, the first pump may generate a pulsed fluid flow and the second pump may generate a continuous fluid flow. Vice versa, the second pump may generate a pulsed fluid flow and the first pump may generate a continuous fluid flow. There may be medical application in which one of these alternatives is preferred compared to two fluid flows of the same kind, i.e. only continuous or only pulsed.

The cannula may be a delivery cannula that is used to guide the fluid flow out of at least one opening of the delivery cannula into the transport volume. Preferably, a drainage cannula may be used to drain the fluid flow from the transport volume using at least one opening of the drainage cannula. The drainage cannula may have an expandable arrangement that isolates the transport volume on a second side of the transport volume, i.e. to complete the isolation of the transport volume form the body fluid system or from the body fluid circuit. Both cannulas may have only one pass through hole within the body and/or at least one expandable arrangement, for instance a balloon or a cage comprising a membrane. Thus, the technical effects that are mentioned above may apply, especially the complete isolation of the transport volume from the body fluid circuit, especially from the blood circuit.

Alternatively, the cannula may be a drainage cannula that is used to drain the fluid flow from the transport volume using at least one opening of the drainage cannula. Preferably, a delivery cannula may be used to guide the fluid flow out of at least one opening of the delivery cannula into the transport volume. The delivery cannula may have an expandable arrangement that isolates the transport volume on a second side of the transport volume, i.e. to complete the isolation of the transport volume form the body fluid system or from the body fluid circuit. Both cannulas may have only one pass-through hole within the body and/or at least one expandable arrangement, for instance a balloon or a cage comprising a membrane. Thus, the technical effects that are mentioned above may apply, especially the complete isolation of the transport volume from the body fluid circuit, especially from the blood circuit.

The cannula may be inserted jugularly. Preferably, a second cannula that is also used to guide the fluid flow is also inserted jugularly. This may allow mobility and higher comfort for a patient if the cannulas are used for more than 24 hours, more than one week or more than one month, e.g. an indwelling cannula/catheter. Furthermore, the circuitry may have a short length. This may allow higher pressures for the fluid flow.

The cannula may be guided at least partially through at least one chamber of a heart, preferably through the left atrium, preferably into both left pulmonary veins, especially if a split tip cannula is used, or into both right pulmonary veins, especially if a split tip cannula is used. Thus new medical applications for lung treatment are possible.

Alternatively, the cannula may be guided at least partially through the right atrium and/or through the right ventricle and preferably into the main pulmonary artery, the left pulmonary artery or the right pulmonary artery. A single tip cannula may be used thereby. New medical application for lung treatment may be feasible using this method.

The cannula may be a single lumen cannula that is easy to use and/or that is simple to produce.

Alternatively, the cannula that is used to perform the methods mentioned above may be a dual-lumen cannula or a multi-lumen cannula which may reduce the complexity of insertion of cannulas and/or allow other medical applications compared to the usage of only single lumen cannulas that are used independently of each other.

The dual-lumen cannula or a multi-lumen cannula may be a fixed dual-lumen or multi lumen cannula, wherein at least two lumens of the cannula are not axially movably with regard to each other. This may simplify the production of the cannula, e.g. there may be less problems with sealing.

Alternatively, the dual-lumen cannula or a multi-lumen cannula may be a non-fixed dual-lumen or multi-lumen cannula, wherein at least two lumens of the cannula are axially movably with regard to each other during use of the cannula, preferably during insertion of the cannula into a body of a subject. The non-fixed dual-lumen or multi-lumen cannula may allow less traumatic insertion compared to a fixed dual-lumen or multi-lumen cannula.

What may be claimed later is a method based on the disclosed methods or on one of its embodiments:

wherein a first multi lumen cannula system is used that comprises a first cannula and a second cannula that is partially arranged within the first cannula,

wherein a second multi lumen cannula system is used that comprises a first cannula and a second cannula that is partially arranged within the first cannula, and

wherein the cannula that is mentioned in claim 24 is the first cannula of the first multi lumen cannula system or the second cannula of the second multi lumen cannula system.

One embodiment that may be claimed later may relate to isolated lung perfusion of the whole lung with heart assist and lung assist, see for instance FIGS. 2A (retrograde flow) and 2B (antegrade flow):

wherein the distal end of the first cannula of the first multi lumen cannula system is placed within the left atrium,

wherein the distal end of the second cannula of the first multi lumen cannula system is placed within the aorta, preferably within the ascending aorta,

wherein a first expandable arrangement is mounted on the distal end portion of the first cannula of the first multi lumen cannula system, preferably covered with a membrane that has an opening that faces laterally and/or that is essentially parallel to two of the cage wires of the diameter variable arrangement,

wherein a second expandable arrangement is mounted on the distal end portion of the second cannula of the first multi lumen cannula system, preferably covered with a membrane that preferably has an opening facing distally and/or that is essentially transversally to the cage wires of the diameter variable arrangement,

wherein the distal end of the first cannula of the second multi lumen cannula system is placed within right atrium or within the right ventricle,

wherein the distal end of the second cannula of the second multi lumen cannula system is placed within the pulmonary artery, preferably within the left pulmonary artery or within the right pulmonary artery, and

wherein a third expandable arrangement is mounted on the distal end portion of the second cannula of the second multi lumen cannula system, preferably covered with a membrane that has preferably an opening facing distally and/or that is essentially transversally to the cage wires of the diameter variable arrangement.

With regard to an embodiment that may be claimed later a retrograde flow may be established within the vessels of the lung, see for instance FIG. 2A. This means that the following flows may be established:

a first flow from a distal end of the second cannula of the second multi lumen cannula system through the second cannula of the second multi lumen cannula out of an outlet port of the second cannula of the second multi lumen cannula system through a first circuitry, preferably comprising a pump, into an inlet port of the first cannula of the first multi lumen cannula system through the first cannula of the first multi lumen cannula system out of distal end of the first cannula of the first multi lumen cannula system, and

a second flow from a distal end of the first cannula of the second multi lumen cannula system through the first cannula of the second multi lumen cannula system through a first circuitry, preferably comprising a group of a pump and an oxygenator device, into an inlet port of the second cannula of the first multi lumen cannula system through the second cannula of the first multi lumen cannula system out of a distal end of the second cannula of the first multi lumen cannula system.

A retrograde flow may have other technical and/or medical effects compared to an antegrade flow. Side vessel between the pulmonary venous system and the bronchial venous system may be exploited to deliver drugs or treatment substance. Switching between retrograde fluid flow and antegrade fluid flow within the transport volume may also be advantageous, for instance to remove blood thrombus/embolism and or to bring fresh treatment substance to all parts of relevant organ tissue, for instance tissue of alveoli of the lung.

With regard to an embodiment that may be claimed later, for instance in a divisional application, an antegrade flow may be established within the vessels of the lung, see for instance FIG. 2B. This means that the following flows may be established:

a first flow from a distal end of the first cannula of the first multi lumen cannula system through the first cannula of the first multi lumen cannula system out of an outlet port of the first cannula of the first multi lumen cannula system through a first circuitry, preferably comprising a pump, into an inlet port of the second cannula of the second multi lumen cannula system through the second cannula of the second multi lumen cannula system out of a distal end of the second cannula of the second multi lumen cannula system, and

a second flow from a distal end of the first cannula of the second multi lumen cannula system through the first cannula of the second multi lumen cannula system through a first circuitry, preferably comprising a group of a pump and an oxygenator device, into an inlet port of the second cannula of the first multi lumen cannula system through the second cannula of the first multi lumen cannula system out of a distal end of the second cannula of the first multi lumen cannula system.

An antegrade flow may have other technical and/or medical effects compared to a retrograde flow, because the natural flow direction is maintained. Switching between retrograde fluid flow and antegrade fluid flow within the transport volume may also be advantageous, for instance to remove blood thrombus/embolism and or to bring fresh treatment substance to all parts of relevant organ tissue, for instance tissue of alveoli of the lung.

With regard to an embodiment (see for instance FIGS. 2D or 2E, antegrade flow) that may be claimed later, for instance in a divisional application, based on any one of the method claims the following may apply:

wherein a multi lumen cannula system is used that comprises a first cannula and a second cannula that is partially arranged within the first cannula,

wherein the second cannula of the first multi lumen cannula system is a delivery cannula for the fluid flow and wherein a single lumen cannula is a drainage cannula for the fluid flow, and

wherein the first cannula of the multi lumen cannula system is used for drainage of blood from the heart, especially from right ventricle and/or from right atrium.

Alternatively, the following may apply (see for instance FIGS. 2D or 2E, retrograde flow):

wherein a multi lumen cannula system is used that comprises a first cannula and a second cannula that is partially arranged within the first cannula,

wherein the second cannula of the first multi lumen cannula system is a drainage cannula for the fluid flow and wherein a single lumen cannula is a delivery cannula for the fluid flow, and

wherein the first cannula of the multi lumen cannula system is used for drainage of blood from the heart, especially from right ventricle and/or from right atrium.

With regard to an embodiment that may be claimed later based on any one of the two alternatives mentioned in the preceding two paragraphs the following may apply:

wherein a further single lumen cannula is used to deliver oxygenated blood into an artery of the body, preferably endovascular femoral, preferably transcaval, see for instance FIGS. 2D and 2E.

With regard to an embodiment that may be claimed later based on the independent method claim the following may apply:

wherein the transport volume is arranged within an organ other than the lung, preferably within the liver or within one of the kidneys or within the stomach or within the liver or within the brain or within an organ of the digestive system, preferably within the colon (for instance in small intestine and/or in large intestine) or within the gall bladder or within the urinary bladder or within the heart (for instance using the coronal vessels to insert the cannula(s)) or within the pancreas.

Further alternatives are the pancreas or other organs mentioned above. The same features may be applied to other organs or parts of organs that are mentioned above for lung or part of lung, for instance partially treatment, partially treatment in sequence of different parts of the same organ and/or retrograde/antegrade treatment.

With regard to an embodiment that may be claimed later based on the previous embodiment the following may apply:

wherein only a part of the organ is treated and wherein the other part of the organ fulfills its natural function during the treatment, and/or

wherein only one part of a pair of preferably essentially identical parts of the same organ is treated and wherein the other part of the organ fulfills its natural function during the treatment, and/or

wherein preferably the treatment of parts of the organ or parts of the pair is changed at least once, twice or more than twice.

With regard to an embodiment that may be claimed later based on the previous embodiment the following may apply:

the fluid flow is directed retrograde and/or wherein the direction of the fluid flow is changed at least once, at least twice or more than twice. The technical and/or medical effects may be similar to the technical and/or medical effects that are mentioned above for the treatment of the lung. Further technical and/or medical effects may be specific to the respective organ, e.g. liver or kidney, etc.

A local treatment of organs or of parts of organs may be possible. Local treatment may allow the usage of high doses of treatment substances that would be otherwise lethal or extremely detrimental.

The transport volume may be limited to only one organ or to only a part of an organ, especially to a pair of essentially identical parts of an organ. Again, a local treatment may be performed, allowing high doses of drugs or treatment substances and/or higher temperatures of the fluid flow and/or higher radiation doses (radioactive radiation, etc.).

The cannula or embodiments of the cannula, the cannula system or its embodiments as well as the kit and the embodiments of the kit may be used to perform the method or its embodiments. Thus, corresponding technical effects may apply. Vice versa, the cannula, the cannula system or the kit and the embodiments of all three devices may have features which are mentioned only for the method. These features may also be used for the devices and may have the same or similar technical effects.

Furthermore, the treatment substance may be applied through at least substance transport channel into a selected region of the lung simultaneously to the fluid flow through the transport volume. Other regions of the lung may be non-selected and may not be in contact with treatment substances. Thus, selective treatment by inhalation or in a different way is possible using for instance air transport channels of the lung to arrange the substance transport channel.

A further aspect relates to method for treating the lung, comprising:

transporting a treatment substance through at least one substance transport channel into a selected region of the lung to treat the selected region,

while treating the selected region transporting a liquid through a transport volume which is separated from a body fluid circuit, for instance the body fluid circuit mentioned above, wherein the selected region is associated with the transport volume.

The treatment substance may be a medicament (pharmaceutical substance) and/or another therapeutic substance. The treatment substance may be applied as aerosols, as a liquid or in another form.

The technical effect may be that comparably high doses may be applied through the substance transport channel, which is different from the blood circuit. At the same time spreading of the medicament or treatment substance may be prevented by using the liquid in the body fluid circuit. The treatment substance may be brought to the outside of the body using the liquid.

The transport volume may be limited by at least one semipermeable membrane of the lung. Additionally or alternatively, the transport volume may comprises tissue of alveoli adjacent to a semipermeable membrane of alveoli of the lung. If the semipermeable membrane and/or the tissue are treated it is possible to locate the treatment substance only to these tissues and to the liquid within the closed transport volume thereby preventing detrimental systemic effects to other body parts.

The at least one substance transport channel may be arranged within at least one air transport channel of the lung. This allows easy application of the treatment substance, e.g. it is only necessary to place a tube through the trachea and then further to the selected region. However, other application ways are also possible.

The method may further comprise:

while treating and/or while transporting the treatment substance through the at least one substance transport channel transporting the liquid through the transport volume, and

while treating and/or transporting the treatment substance through the at least one substance transport channel transporting the liquid to the outside of a body of which the lung is part of.

Thus, there is a time relationship between treatment and transporting of the liquid, e.g. both are uses simultaneously or within one hour or within several hours.

The treatment substance may be transported to only one half of the lung, to only one lobe of the lung or to only a part of a lobe of the lung. Local treatment of the lung enables to treat only tissue which needs a treatment. Healthy tissue may not be treated or not in an unnecessary manner Furthermore, the healthy tissue or tissue which is not treated, at least not in the moment, may be used to fulfill the normal functions of the lung, e.g. carbon dioxide removal from the blood and/or oxygen enhancement in the blood. The transport volume may be restricted to regions which are treated at the moment but not to healthy or untreated regions of the lung.

There are for instance the following possibilities for local treatment of the lung:

a) the treatment substance is transported only to a part or to all alveoli in one selected half of the lung but not to non-selected alveoli in a non-selected half of the lung and wherein the liquid is transported in an essentially closed or in a closed transport volume at least through the tissue of the selected alveoli in the selected half of the lung but not to alveoli in the non-selected half of the lung, or

b) the treatment substance is transported only to alveoli in one selected lobe of a selected half of the lung but not to non-selected lobes of the selected half of the lung or only to alveoli in some selected lobes of the selected half of the lung but not to a non-selected lobe in the selected half of the lung and wherein the liquid is transported in an essentially closed or in closed transport volume at least through the tissue of the alveoli in the selected lobe or in the selected lobes but not through the tissue of alveoli in a non-selected half of the lung or in the non-selected lobes of the lung, or

c) the treatment substance is transported only to a first part of the alveoli of a selected lobe of a selected half of the lung but not to a second part of alveoli of the selected lobe and not to alveoli in a non-selected half of the lung and wherein the liquid is transported in an essentially closed or in a closed transport volume at least through the tissue of the first part of the alveoli in the selected half of the lung but not through the tissue of alveoli in the non-selected half of the lung of through the tissue of alveoli in the non-selected lobes of the lung.

The method may be performed using a cannula according to any one of the embodiments mentioned above or using a cannula system according to any one of the embodiments mentioned above for transporting the liquid. Furthermore, the method according to the further aspect may be combined with the method (fourth aspect) according to any one of the embodiments mentioned above.

The treatment substance may comprise stem cells of at least one kind of tissue in the lung or a medicament against lung cancer or a medicament against a lung infection. Stem cell therapy is possible because the stem cells may be applied locally, e.g. growth of the stem cells in other organs will not take place.

The treatment substance or at least a part of the treatment substance may be removed from the liquid. The cleaned liquid may be transported back into the body, preferably back into the transport volume. Thus, limited resources of blood may be used in an efficient way. However, if replacement of the liquid is possible cleaning may not be necessary.

A further aspect relates to a further kit for defining at least one border of a transport volume for an in-vivo fluid transport, comprising:

a cannula as mentioned above, and/or

a cannula system as mentioned above, and:

a transport device comprising at least one substance transport channel for transporting a treatment substance preferably to only a selected region of the lung,

wherein preferably the substance transport channel may be adapted to be arranged at least partially or at least with 90 percent of its length in an air transport channel of the lung,

wherein the transport device may have at least one port outside of the lung and at least one port inside of the lung.

The kit may comprise at least one pump for driving a fluid flow through the transport volume, preferably a roller pump or a membrane pump or a centrifugal pump or a diagonal pump or an axial pump.

Additionally or alternatively the kit may comprise a delivery unit for the introduction of the at least one (first) treatment substance into the fluid flow in order to guide the treatment substance and/or a further (second) treatment substance into the transport volume via the fluid flow. Moreover, the kit may comprise a removing device for removing the treatment substance (first) and/or the further treatment substance (second) from the fluid flow.

The kit may be used for performing a method according to any one of the embodiments mentioned above for the further method aspect.

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

The term “haemoperfusion/hemoperfusion”, as used herein, may refer to a method of filtering blood extracorporeally (that is, outside the body) to remove one or more toxins. As with other extracorporeal methods, such as hemodialysis (HD), hemofiltration (HF), and hemodiafiltration (HDF), the blood travels from the patient into a machine, gets filtered, and then travels back into the patient, typically by venovenous access (out of a vein and back into a vein). During the extracorporeal process, inflammatory and other harmful molecules are removed from the blood of the organism of a patient. Adsorbers (filters), in particular macroporous resin beads, are preferably used, which allow an effective blood purification. These adsorbers in turn may be combined with other therapy components, which regulate the metabolism and strengthen the immune system. In this way, drug strategies can take effect more effectively. Alternatively and/or additionally absorber techniques may be used. However, the filtering may alternatively concern a fluid flow that does not contain blood or that does not contain blood as the main part, i.e. it contains blood or blood components only by 50 percent per volume or less than 50 volume percent.

The term “hyperoxygenated haemoperfusion/hemoperfusion”, as used herein, may refer to the haemoperfusion/hemoperfusion as described above but extended by an oxygenator that generates an oxygen level in the blood or in the fluid flow that is used for treatment that is higher than a normal oxygen level within blood, for instance if the body rests. Thus the oxygen content of the blood may be increased, for instance at least by 5 percent or at least by 10 percent. The oxygen content of body tissue may be increased even more, for instance by more than 10 percent or more than 50 percent compared for instance to a normal oxygen level, for instance if the body rests. Hyperoxygenation may have a positive effect for the uptake of medicaments and/or treatment substances by the body or more specific by the organ that is under treatment.

The term “hypooxygenated haemoperfusion/hemoperfusion”, as used herein, may refer to the haemoperfusion/hemoperfusion as described above but extended by an oxygenator and/or a carbon dioxide removal or introduction unit that generates an oxygen level that is lower than the normal oxygen level within blood, for instance if the body rests. This may reduce the oxygen content of the blood and/or of body tissue, for instance by more than 10 percent or by more than 50 percent. Hypooxygenation may have a positive effect for the uptake of medicaments and/or treatment substances by the body or more specific by the organ that is under treatment. Furthermore, there may be medicaments and/or treatment substances that react with oxygen. This reaction may be detrimental for the therapeutic effect and it may be advantageous to prevent or reduce the reaction as far as possible.

The term “disease”, as used herein, may refer to an abnormal condition that affects the body of an individual. A disease is often understood as a medical condition associated with specific symptoms and signs. A disease may be caused by factors originally from an external source, such as infectious disease, or it may be caused by internal dysfunctions, such as autoimmune disease. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infectious, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality. In the context of the present invention, the disease may be selected from the group consisting of or comprising cancer and infectious disease.

The terms “cancer disease” or “cancer”, as used herein, may refer to or describe the physiological condition in an individual that is typically characterized by unregulated cell growth. Examples of cancers include, but are not limited to, carcinoma, lymphoma, blastoma and sarcoma. More particularly, examples of such cancers include lung cancer, liver cancer or cancer of other organs.

The terms “individual” and “subject” can be used interchangeable herein. The individual or subject may be any mammal, including both a human and another mammal, e g an animal Human individuals or subjects are particularly preferred. The individual may be a patient.

The term “patient”, as used herein, may refer to any subject suffering from a disease, in particular suffering from cancer, an autoimmune disease, and/or infectious disease. The patient may be treated and/or the response to said treatment may be evaluated. The patient may be any mammal, including both a human and another mammal, e g an animal Human subjects as patients are particularly preferred.

The term “treatment”, in particular “therapeutic treatment”, as used herein, refers to any therapy which improves the health status and/or prolongs (increases) the lifespan of a patient. Said therapy may eliminate the disease in a patient, arrest or slow the development of a disease in a patient, inhibit or slow the development of a disease in a patient, decrease the frequency or severity of symptoms in a patient, and/or decrease the recurrence in a patient who currently has or who previously has had a disease.

A drug used in chemotherapy is a chemotherapeutic agent. The term “chemotherapeutic agent”, as used herein, may refer to a compound that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. The chemotherapeutic agent is preferably selected from the group consisting of alkylating agents, anthracyclines, cytoskeletal disruptors (taxanes), epothilones, histone deacetylase inhibitors, inhibitors of topoisomerase I, inhibitors of topoisomerase II, kinase inhibitors, nucleotide analogs and precursor analogs, peptide antibiotics, platinium-based agents, retinoids and vinca alkaloids and its derivatives. Liquid drugs may be used.

However, usage of small balls or beads may be advantageous to deliver the medicament and/or the therapeutic substance, for instance usage of nanoballs or nanoparticles or of micro particles Liposomes may be used as nano particles or as micro particles.

The term “radiation therapy (also called radiotherapy)”, as used herein, may refer to a cancer treatment that uses high doses of radiation to kill cancer cells and shrink tumors. At low doses, radiation is used in X-rays to see inside the body. At high doses, radiation therapy kills cancer cells or slows their growth by damaging their DNA. Cancer cells whose DNA is damaged beyond repair stop dividing or die. When the damaged cells die, they are broken down and removed from the body. Radiation therapy may not kill cancer cells right away. It may take days or weeks of treatment before DNA may be damaged enough for cancer cells to die. Then, cancer cells may keep dying for weeks or months after radiation therapy ends. Classical radiation by using ionizing radiation or electronic X-ray devices may be used. Alternatively, radioactive radiation may be used or radioactive substances may be brought into contact with the body, specifically with the treated organ or with a part of treated organ.

However, usage of small balls or beads may be advantageous to bring radioactive substances into the body, for instance usage of nanoballs or nanoparticles. Liposomes may be used to produce the nanoparticles or the micro particles.

The term “extracorporeal blood”, as used herein, may refer to blood removed/isolated from an individual's blood circulation.

The term “extracorporeal circuit”, as used herein, may refer to a procedure in which blood is taken from an individual's circulation to have a process applied to it before it is returned to the circulation. All of the system carrying the blood outside the body is termed the extracorporeal circuit.

The term “systemic administration”, a used herein, may refer to the administration of the therapeutic agent, e.g. chemotherapeutic agent, such that said agent becomes widely distributed in the body of a patient in significant amounts and develops a biological effect. Typical systemic routes of administration include administration by introducing the therapeutic agent, e.g. chemotherapeutic agent, directly into the vascular system or oral, pulmonary, or intramuscular administration wherein the therapeutic agent, e.g. the chemotherapeutic agent, enters the vascular system and is carried to one or more desired site(s) of action via the blood. The systemic administration may be by parenteral administration. However, the proposed cannulas and methods may be especially advantageous for more local treatment of organs or of parts of organs.

The term “parenteral administration”, as used herein, may refer to the administration of the therapeutic agent, e.g. chemotherapeutic agent, such that said compound does not pass the intestine. The term “parenteral administration” includes intravascular administration, intravenous administration, subcutaneous administration, intradermal administration, or intraarterial administration, but is not limited thereto.

It is also preferred that the extracorporeal blood is oxygenated blood. Preferably, the extracorporeal blood has been oxygenated by external means, e.g. using an oxygenator. The oxygenator enhances oxygen within the blood. Alternatively, the oxygen content of blood may be reduced below a normal level. However, normal oxygen levels within blood or other fluids may be used as well.

It is further preferred that the blood is purified blood. Extracorporeal blood purification (EBP) is a treatment in which a patient's/donor's blood is passed through a device (e.g. membrane, sorbent) in which solute (e.g. waste products, toxins) and possibly also water are removed. When fluid is removed, replacement fluid is usually added. It is preferred that purified blood does not comprise inflammatory, toxic molecules and/or other harmful molecules anymore or comprises a reduced amount of said molecules compared to unpurified blood. Extracorporeal therapies designed to remove or filter substances from the circulation in order to purify blood include hemodialysis, hemofiltration, hemoadsorption, plasma filtration, cell-based therapies and combinations of any of the above. Preferably, the purified blood is filtered blood. More preferably, the purified blood is dialyzed blood. Blood dialysis removes waste, salt, toxins, extra water to prevent them from building up in the body, keeps a safe level of certain chemicals in the blood such as potassium, sodium, and bicarbonate, and helps to control blood pressure. It is particularly preferred that the blood, e.g. the purified, filtered, or dialyzed blood, is free of inflammatory or other harmful molecules. The same features may apply to other transport fluids than blood.

It is further preferred that the cancer is selected from the group comprising or consisting of, lung cancer, urothelial cancer, bladder cancer, liver cancer, kidney cancer/renal cancer, stomach cancer, brain cancer.

It is further preferred that the chemotherapeutic agent is selected from the group comprising or consisting of alkylating agents, anthracyclines, cytoskeletal disruptors (taxanes), epothilones, histone deacetylase inhibitors, inhibitors of topoisomerase I, inhibitors of topoisomerase II, kinase inhibitors, nucleotide analogs and precursor analogs, peptide antibiotics, platinium-based agents, retinoids vinca alkaloids and its derivatives.

Systemic routes and/or local routes of administration may include administration by introducing the chemotherapeutic agent directly into the vascular system or pulmonary wherein the chemotherapeutic agent enters the vascular system and is carried to one or more desired site(s) of action. This is described in more detail below.

In particular, the chemotherapeutic agent may be suitable to be administered topically, intravenously, intraarterially, intrapleurally, by inhalation, via a catheter. The dose or more specifically the size of the dose which can be administered to a patient (“a therapeutically effective amount” or simply “an effective amount”) should be sufficient to effect a beneficial therapeutic response in the patient over time. The dose or more specifically the size of the dose will be determined by the efficacy of the particular chemotherapeutic agent employed and the condition of the patient, as well as the body weight or surface area of the patient to be treated. The size of the dose also will be determined by the existence, nature, and extent of any adverse side-effects that accompany the administration of the chemotherapeutic agent in a particular patient. The proposed invention may reduce adverse side-effects tremendously as the chemotherapeutic agent may be applied only locally and isolated from other body fluid circuits, especially form the blood circulation circuit of the body.

The chemotherapeutic agent may be administered in higher concentrations or even in much higher concentrations compared to systemic administration, i.e. to the whole body.

The proposed method and its embodiments may not be used for treatment of the human or animal body by surgery or therapy and may not be a diagnostic method practiced on the human or animal body. Alternatively, the proposed method and its embodiments may be used for treatment of the human or animal body by surgery or therapy and may be a diagnostic method practiced on the human or animal body.

The making and using of the presently preferred embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims.

Moreover, same reference signs refer to the same technical features if not stated otherwise. As far as “may” is used in this application it means the possibility of doing so as well as the actual technical implementation. The present concepts of the present disclosure will be described with respect to preferred embodiments below in a more specific context namely heart and lung surgery. The disclosed concepts may also be applied, however, to other situations and/or arrangements in heart and/or lung surgery as well, especially to surgery of other organs.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

For a more complete understanding of the presently disclosed concepts and the advantages thereof, reference is now made to the following description in conjunction with the accompanying drawings. The drawings are not drawn to scale. In the drawings the following is shown in:

FIG 1A an extra corporeal circular lung perfusion blood flow circuitry comprising two single lumen cannulas, a pump and at least on further device,

FIG. 1B an embodiment of pIVLP (percutaneous in-vivo lung perfusion) that may be used for instance with low fluid flow,

FIG. 1C a further embodiment of pIVLP (percutaneous in-vivo lung perfusion) that may be used for instance with middle fluid flow,

FIG. 2A an extra corporeal retrograde lung perfusion circular blood flow circuitry comprising two dual lumen cannulas,

FIG. 2B an alternative embodiment with antegrade lung perfusion,

FIG. 2C an alternative embodiment with lobe dedicated lung perfusion and an embodiment for right ventricle assist,

FIG. 2D a further embodiment of pIVLP (percutaneous in-vivo lung perfusion) that may be used for instance with high fluid flow and with blood oxygenation,

FIG. 2E a further embodiment of pIVLP (percutaneous in-vivo lung perfusion) that may be used for instance with high double fluid flow and optional inhalation.

FIG. 3 a cannula system having cannulas that are arranged coaxially,

FIG. 4 a cannula system having an inner (second) cannula that is arranged loosely within an outer (first) cannula,

FIG. 5 a cross section of another cannula system,

FIG. 6 an embodiment of a dual lumen system comprising at least one pre-bended cannula,

FIG. 7 a cage arrangement comprising a membrane having an opening that faces distally,

FIG. 8 a cage arrangement comprising a membrane having an opening that faces laterally,

FIG. 9 a cage arrangement comprising a portion that is bended backwards and that comprises no membrane,

FIG. 10 a cannula comprising a cage arrangement having wires that are arranged in parallel with regard to each other,

FIG. 11 a cannula comprising a cage arrangement having a cone like shape,

FIG. 12 the cannula of FIG. 11 in a state in which an introducer member stretches the cage arrangement for introducing the cannula into a body,

FIG. 13 an alternative embodiment wherein a cannula is pierced or punctured through a ventricle septum,

FIG. 14 a further alternative embodiment wherein a cannula is pierced or punctured through a ventricle septum,

FIG. 15 an alternative embodiment wherein a cannula is punctured transcaval from vena cava to the aorta,

FIG. 16 a further alternative embodiment wherein a cannula is punctured transcaval from vena cava to the pulmonary artery,

FIG. 17 a cannula that carries an inflatable expandable arrangement,

FIG. 18 a split tip cannula that carries two expandable arrangements, and

FIG. 19 an embodiment of a lung perfusion system in combination with an inhalation system for treating only one lobe of the lung.

### DESCRIPTION OF FIGURES:

The heart H of a patient is located within his or her thorax. The patient may be a male or female adult or a child. The heart H comprises the following chambers:
right atrium RA,
right ventricle RV,
left atrium LA, and
left ventricle LV.

The atrial septum AS is between right atrium RA and left atrium LA. The ventricle septum VS is between right ventricle RV and left ventricle LV.

The following valves of heart H are shown in the following figures:
tricuspid valve TVa between right atrium RA and right ventricle RV, and
mitral valve MVa between left atrium LA and left ventricle LV,—aortic valve AOV is between aorta AO and left ventricle LV, and
pulmonary valve PV between right ventricle RV and pulmonary artery PA.

There are two left pulmonary vein IPV and two right pulmonary veins that extend into the left atrium LA of the heart H. Blood that is enriched with oxygen comes from lung L into left atrium LA through pulmonary veins PV. This is an exception in that a vein transports blood that comprises more oxygen than blood in a comparable artery. The description of heart H will not be repeated below. However, it is clear that this description is valid for all Figures that show a heart H.

An isolation of lung L is reached possibly together with heart assist of heart H at the same moment for the circuitries that use percutaneous in-vivo lung perfusion (pIVLP). Thus, isolated perfusion and/or treatment of lung L diseases is enabled, especially antegrade fluid flow and/or retrograde fluid flow, preferably also with switching between antegrade and retrograde or between retrograde and antegrade flow. However, if only a part of lung L is treated, the other part may function normal. There may be a lobe dedicated treatment or treatment of only a part of a lobe of lung L. This may allow to treat lung L without heart H assist/support and or without lung L support, e.g. without external blood oxygenation and/or without external carbon dioxide (CO₂) removal. Alternatively, partially or full heart H assist and/or lung L assist may be used even if only a part of the lung is treated.

FIG. 1A illustrates an extra corporeal lung perfusion circular blood flow circuitry 906 comprising two single lumen cannulas 910 and 940, a pump P9 and at least one further device D9. Single lumen cannula 910 carries a cage arrangement 916 near at least one inlet port that is arranged in left atrium LA. Second single lumen cannula 940 has at least one outlet port within pulmonary artery PA. If the whole lung L is treated, circuitry 906 is a more theoretical embodiment because in addition a heart assist would be necessary. However, there are many ways to realize such a heart assist. One possibility is described below with reference to FIGS. 2A to 2E. However, if only a part of the lung is treated no heart H assist and/or lung L assist may be necessary.

Cannula 910 is inserted endovascularly through the left internal jugular vein IJV, superior vena cava SVC, right atrium RA, trans-septal, i.e. through the atrial septum AS between right atrium RA and left atrium LA and into left atrium LA. A guide wire (not shown) may be used to guide cannula 910 to its final position. Alternatively, cannula 910 may be inserted through the right subclavian vein. Almost the whole blood that enters left atrium LA through the left pair of pulmonary veins PV, i.e. left pulmonary veins IPV, and through the right pair of pulmonary veins PV, i.e. right pulmonary veins rPV, may be taken in by cannula 910, see arrow 960, using a membrane 919 that is explained in more detail below.

The second single lumen cannula 940 is inserted endovascularly through the right internal jugular vein IJV, superior vena cava SVC, right atrium RA, right ventricle RV, through pulmonary valve PVa into pulmonary artery PA. A guide wire (not shown) may be used to guide cannula 940 to its final position. Alternatively, cannula 940 may be inserted through the right subclavian vein and then along the same way as described above. Almost the whole blood that comes out of cannula 940 is injected into pulmonary artery PA, see arrow 970, using a membrane 949 that is explained in more detail below. Device D9 may be an injection device that injects a medicament or a treatment substance, for instance for treating lung L cancer.

An optional inlet tip 914 may be mounted on distal end 912 of cannula 910. Inlet tip 914 may comprise a plurality of inlet holes 915 in its side wall. Additionally, there may be a hole within the distal end of inlet tip 914. The sum of the cross section areas of the holes of tip 914 may be greater than the inner cross section area of cannula 910 at its distal end 912, for instance greater than twice the area or the triple of the area. This means that blood can be removed even if one or more of inlet holes 915 in inlet tip 914 is or are clogged.

However, in other embodiments no inlet tip 914 is used. Thus, there is only one inlet hole at distal end 912 of cannula 910. This single inlet hole (end-hole) would be surrounded by cage arrangement 916. Using a cannula without a separate tip allows high flow rates of a fluid that is drained into the cannula 910. The cage arrangement 916 prevents that a wall of left atrium LA is sucked into the end-hole of cannula 910.

Cage arrangement 916 is one possible example. Other possible examples are described below with reference to FIGS. 7 to 12. Cage arrangement 916 may comprise for instance between 6 to 12 flexible wires, beams or bars. There may be for instance 8 cage wires 918 that span a sphere. The sphere prevents that the side wall of the left atrium LA covers one of inlet holes 915 of tip 914. Furthermore, cage arrangement 916 fixes and/or places distal end 912 of cannula 910 to the atrial septum AS. Thus it is not possible that cannula 910 slides back through the atrial septum AS into right atrium RA.

Membrane 919 may cover only one half of cage arrangement 916, e.g. a half that is defined by two cage wires 918 that are arranged opposite to each other or nearly opposite. An examples of a membrane that may be used on cage arrangement 916 is described below with reference to FIG. 8.

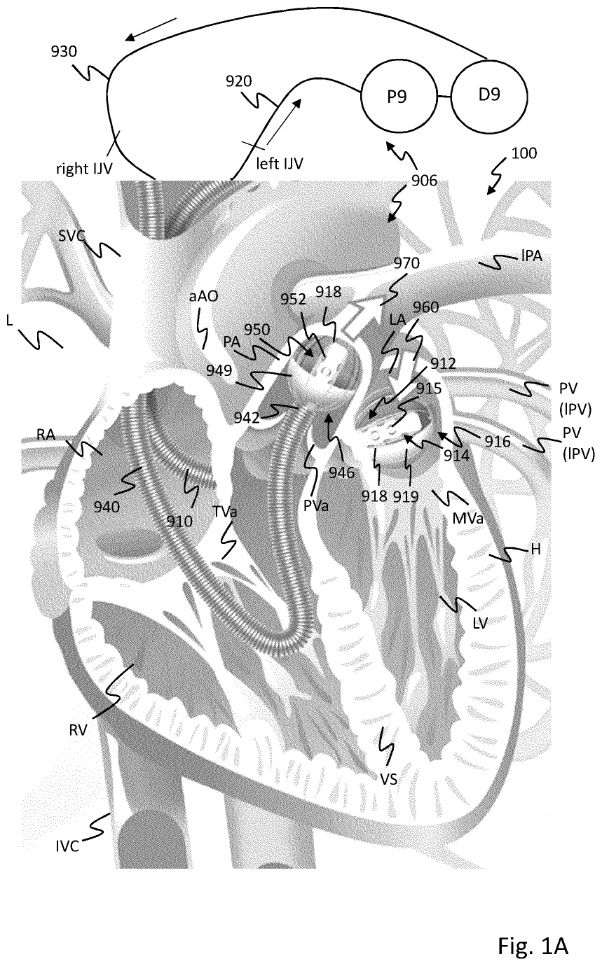

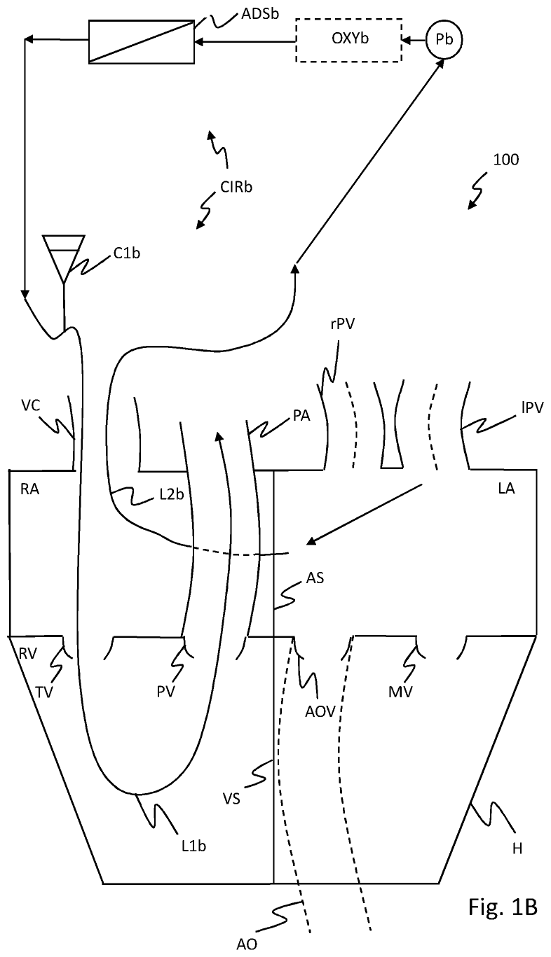

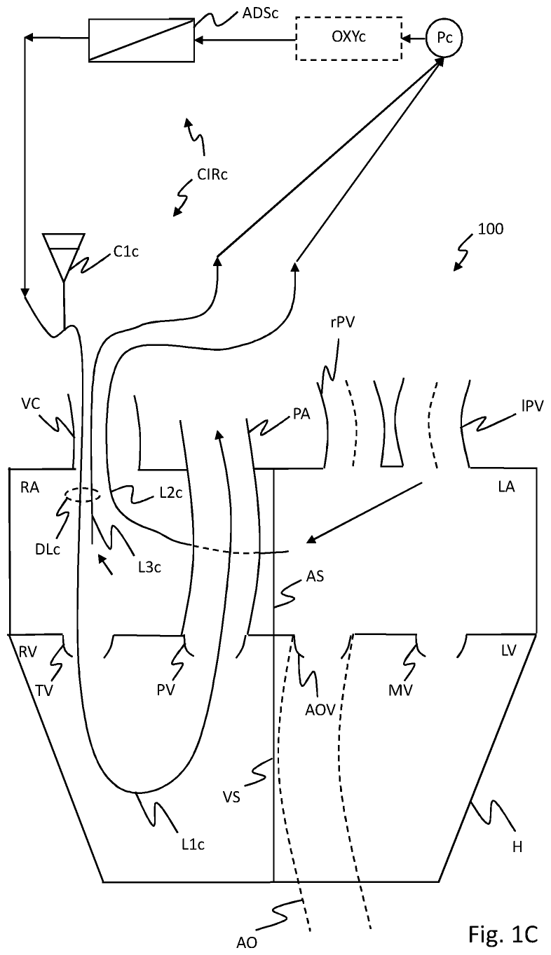

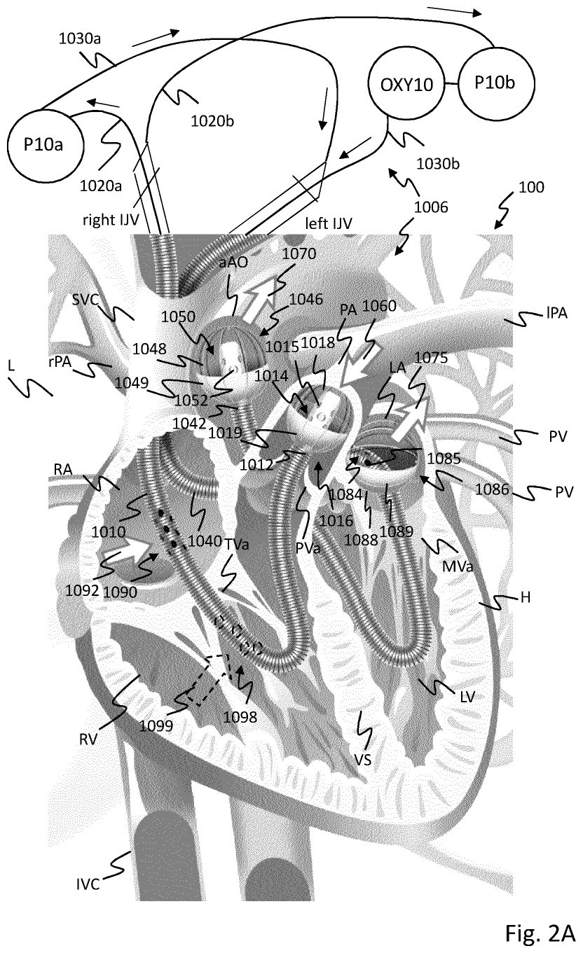

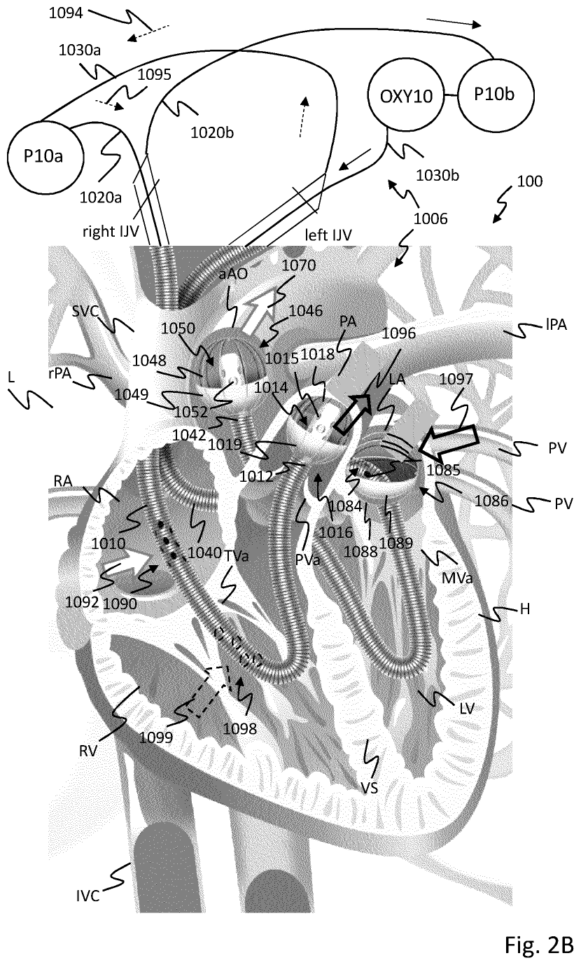

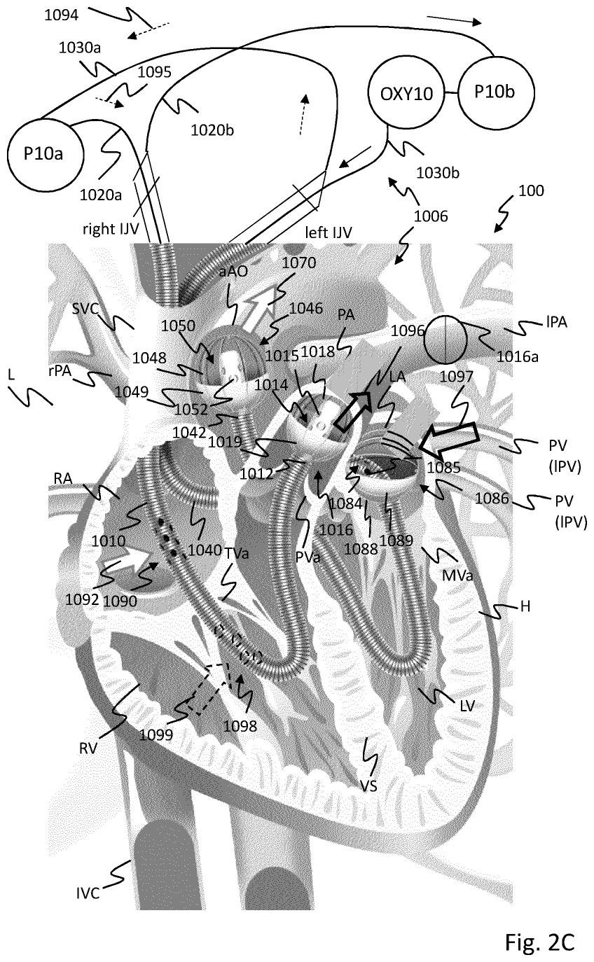

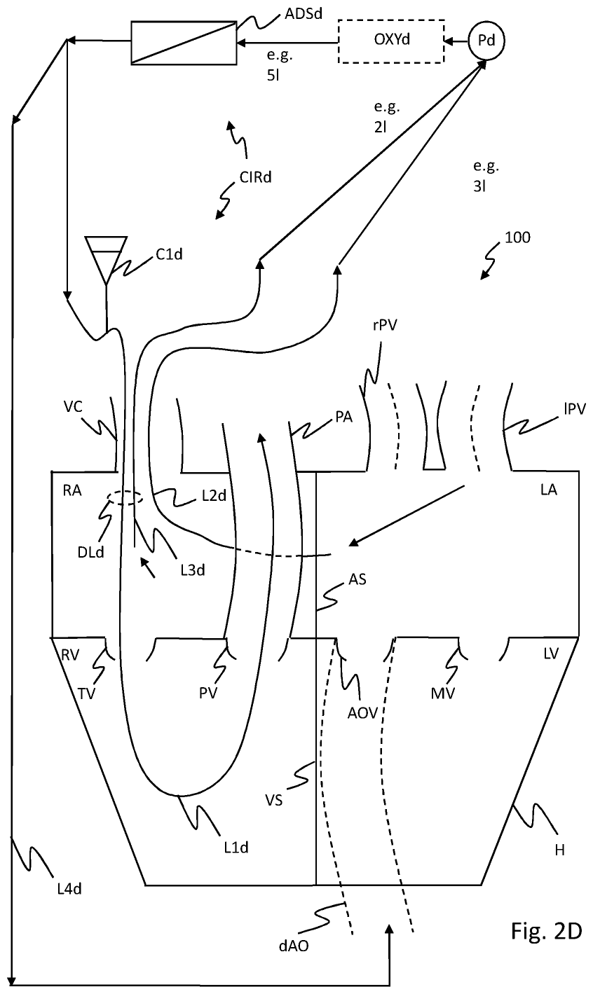

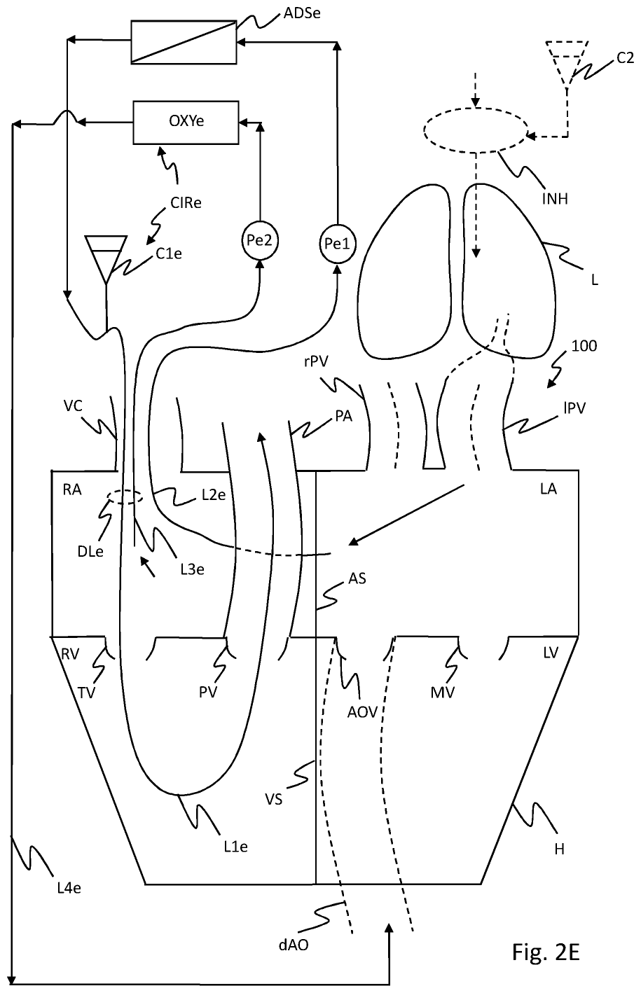

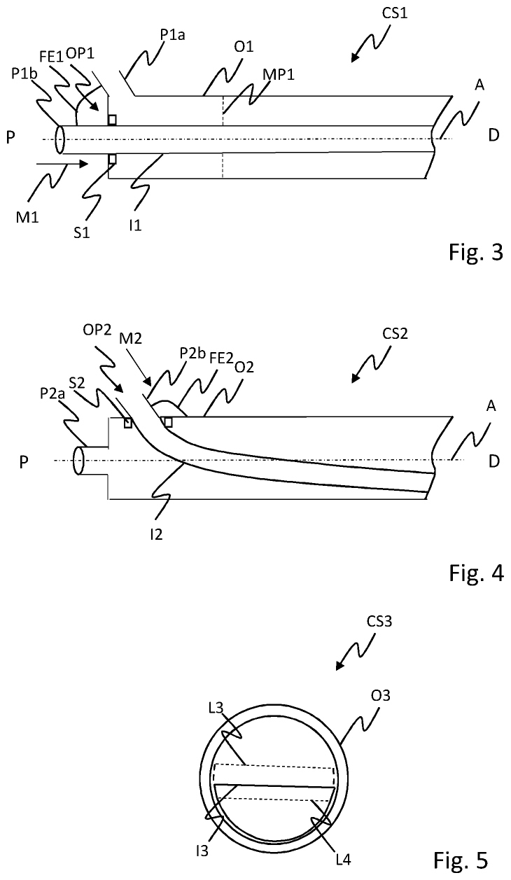

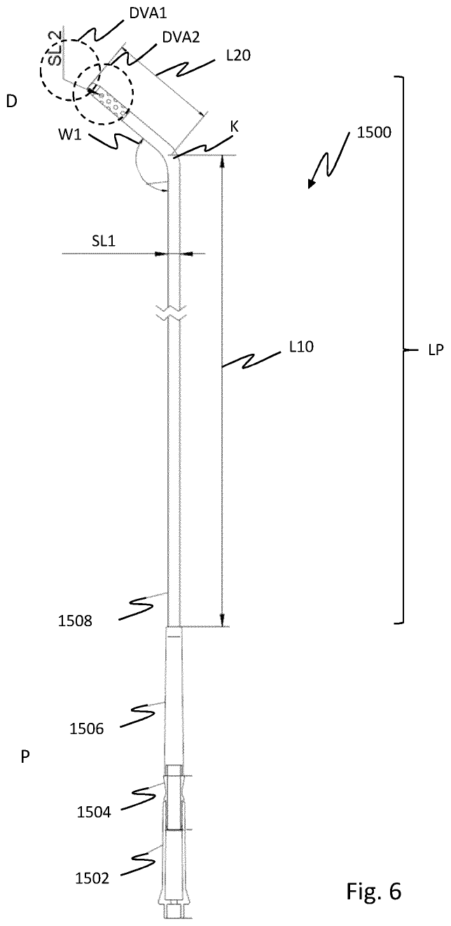

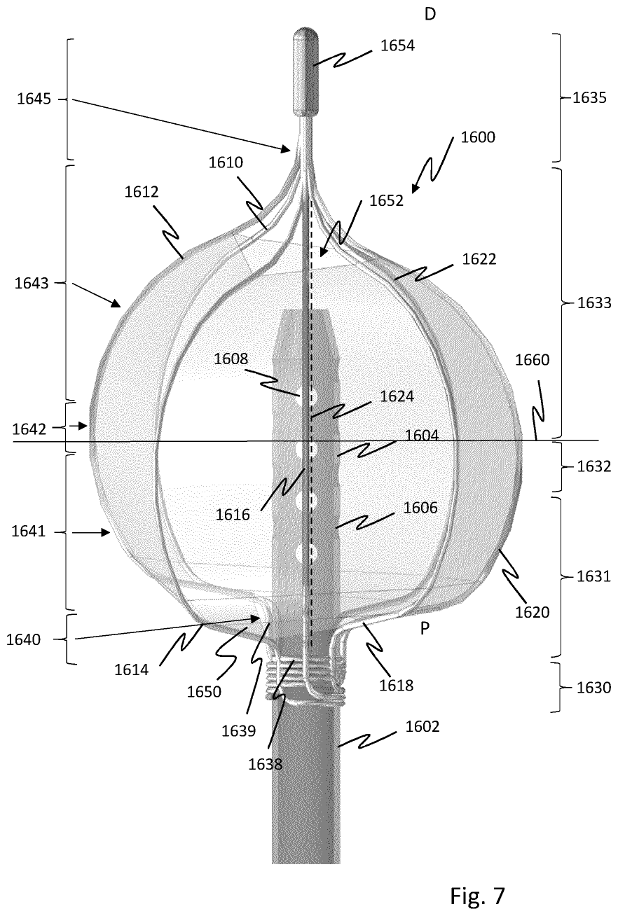

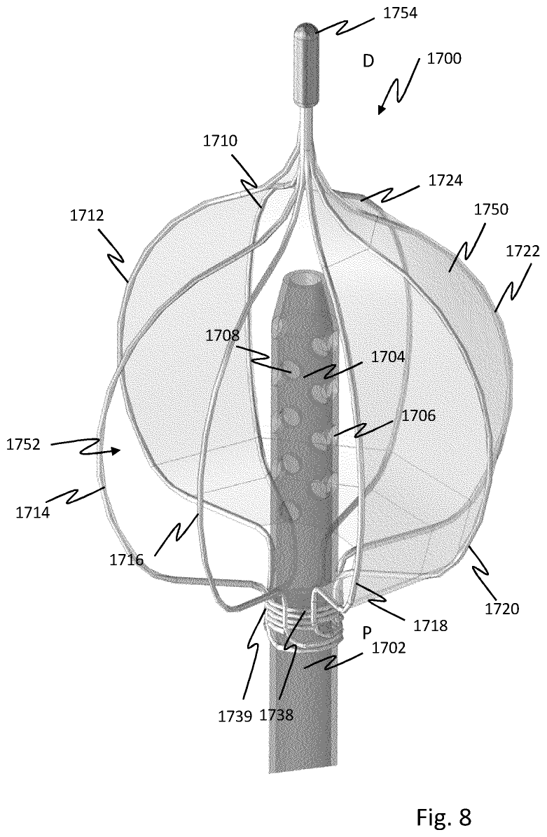

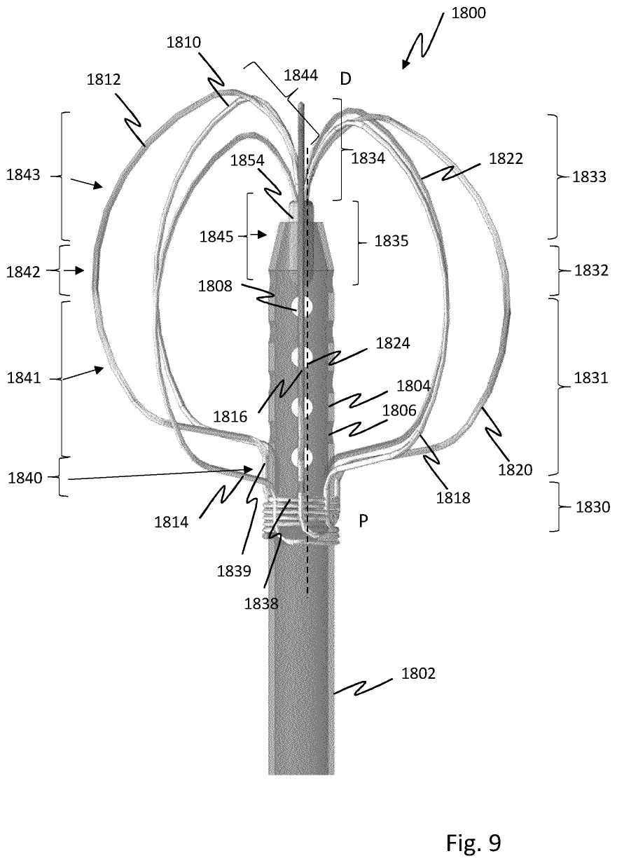

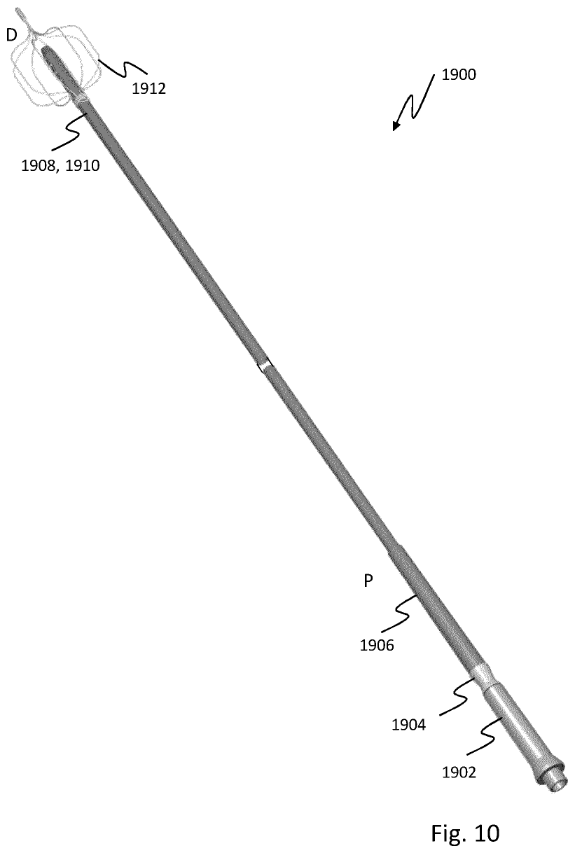

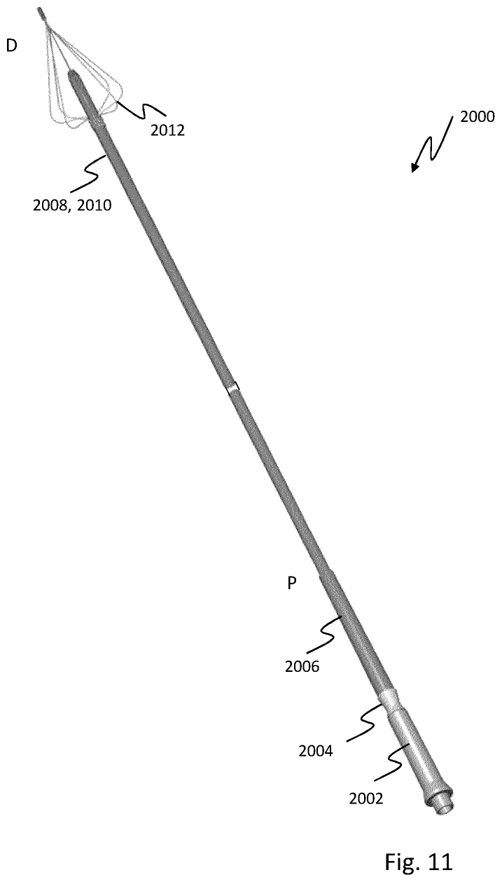

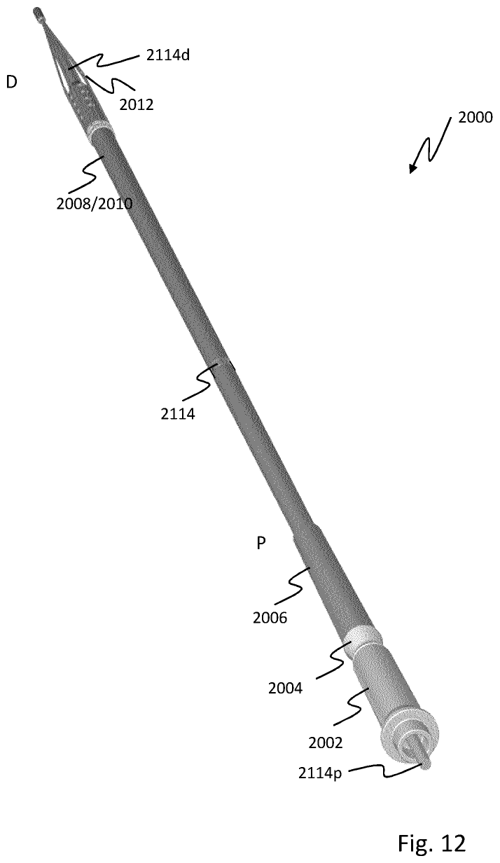

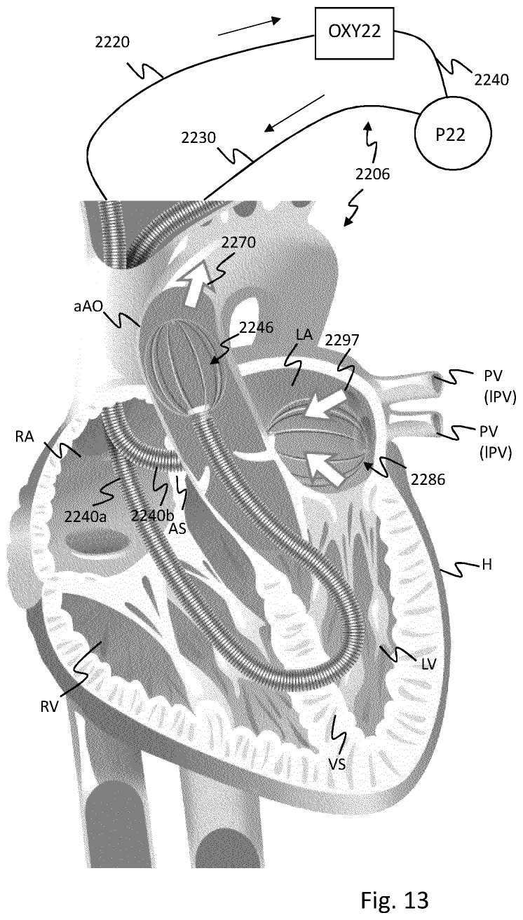

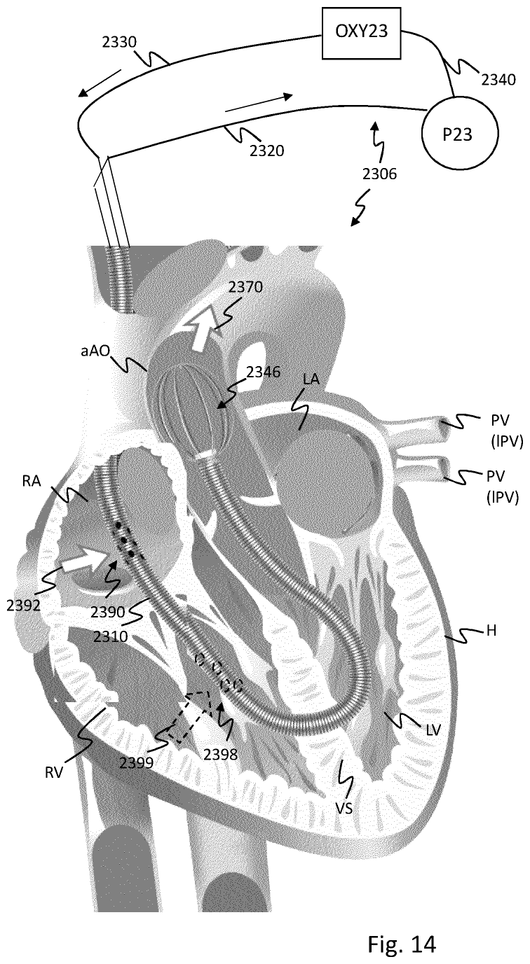

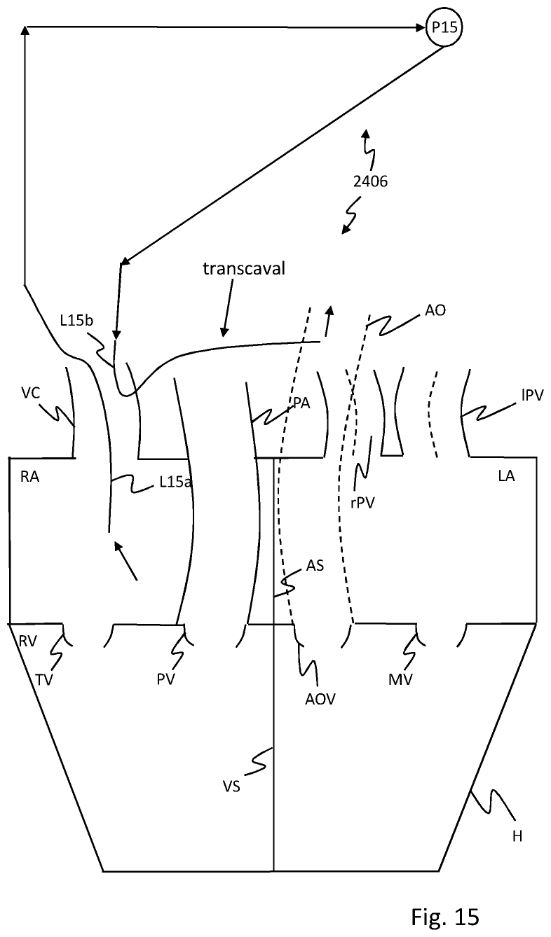

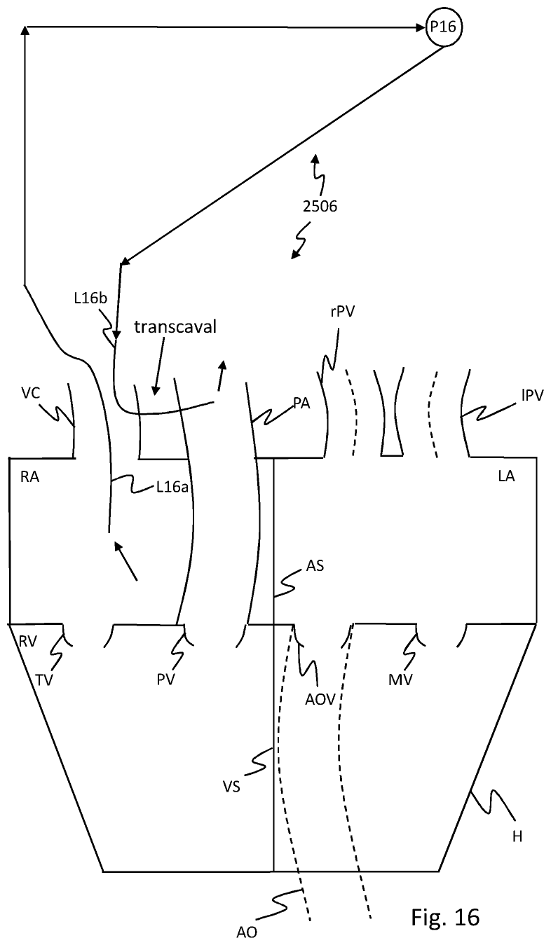

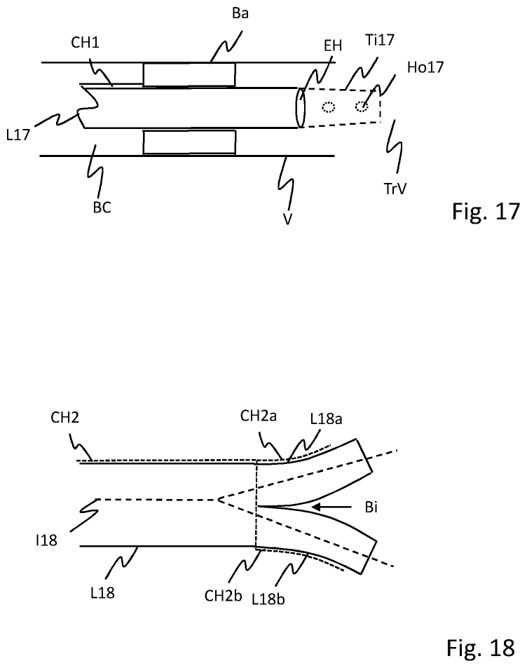

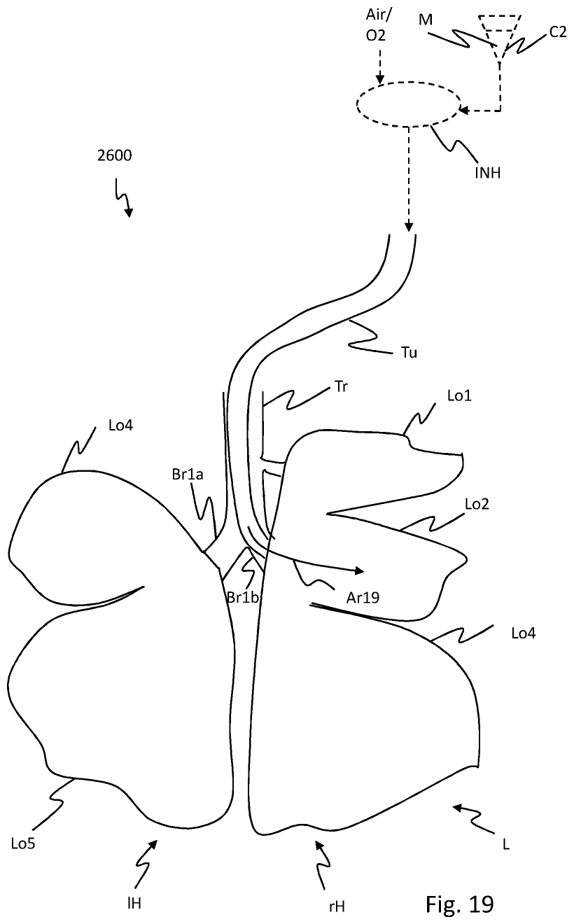

Further to FIG. 1A, a tube 920 is connected to a proximal end of cannula 910 and to an inlet of pump P9. An outlet of pump P9 may be connected to an inlet of device D9. A tube 930 is connected to an outlet of device D9 and to the proximal end of cannula 940. Device D9 may be used for instance for injecting a drug or medicament or a treatment substance into lung L of the patient.

Tubes 920, 930 may be made of a flexible material or of a more rigid material. The circuitry 906 may further include one or more blood filter units or units for dialysis of blood.

Cannula 940 may comprise an optional outlet tip 950 that may have the same or similar structure as inlet tip 914 of cannula 910. This means that outlet tip 950 may comprise a plurality of outlet holes 952 in its side wall and/or on its distal end. Additionally, cannula 940 may have an optional cage arrangement 946 on its distal end 942.

However, in other embodiments no outlet tip 950 is used. Thus, there is only one inlet hole (end-hole) at distal end 942 of cannula 940. This single inlet hole may be surrounded by cage arrangement 946.

Cage arrangement 946 is one possible example. Other possible examples are described below with reference to FIGS. 7 to 12. Cage arrangement 946 may comprise for instance between 6 to 12 flexible wires, beams or bars. There may be for instance 8 cage wires 948 that span a sphere. The sphere and also the expelled blood prevent that the side wall of pulmonary artery PA covers one of outlet holes 952 of optional outlet tip 950. Furthermore, cage arrangement 946 fixes distal end 942 of cannula 940 within pulmonary artery PA.

Membrane 949 may cover only one half of cage arrangement 946, e.g. a half that is between the distal end of cannula 940 and the mid of cage wires 948. An example of a membrane that may be used on cage arrangement 946 is described below with reference to FIGS. 7 to 12.

No extra care has to be taken because both cannulas 910 and 940 are inserted into veins in which there is comparably low blood pressure compared to the blood pressure in arteries. Antegrade infusion is performed into pulmonary artery PA that has many advantages because it corresponds to the natural direction of blood flow in lung L of the patient.

The arrangement shown in FIG. 1A may be used for patients with lung L problems. Mobility of the patient is possible because no cannulas are used in femoral veins or arteries. The arrangement shown in FIG. 1A may be named pIVLP (percutaneous in vivo lung perfusion).

In other embodiments it is possible to insert cannula 910 through right internal jugular vein IJV or right subclavian vein to left atrium LA as described above and cannula 940 through left internal jugular vein IJV or left subclavian vein to left atrium LA.

Alternatively and/or additionally, device D9 may be or may comprise a CO₂ (carbon dioxide) removal device, an oxygenator, etc.

Furthermore, the pumping direction may be directed reverse in each application scenario that is mentioned above, i.e. retrograde instead of antegrade. Furthermore, it is possible to switch direction of the fluid flow once or several times.

During treatment of the lung L it is possible that the patient inhales a medicament or treatment substance in order to promote the treatment by the substance or medicament that flows through the vessels of the lung L and through the tissue of the alveoli. The fluid flow within circuitry 906 may comprise blood as a carrier substance. Alternatively other carrier substances may be used, for instance based on saline and/or on water.

FIG. 1B illustrates an embodiment of a pIVLP (percutaneous in-vivo lung perfusion) circuitry CIRb that may be used for instance with comparably low fluid flow of for instance 0.5 liter per minute (L/min) to 1 liter per minute). Circuitry CIRb may comprise or consist of:

a single lumen cannula L1b,

a further single lumen cannula L2b,

a pump Pb,

an adsorber/filter unit ADSb,

a treatment substance delivery unit C1b, and

an optional oxygenator OXYb and/or carbon dioxide removal unit.

Single lumen cannula L1b may be inserted endovascularly through the right internal jugular vein rIJV or the left internal jugular vein WV, through superior vena cava SVC, right atrium RA, left ventricle LV and into pulmonary artery PA. An introducer member and/or a guide wire may be used to enable and/or to ease insertion of cannula L1b. The maximum outer diameter of cannula L1b may be for instance 18 F (French), 19 F or 21 F.

Single lumen cannula L1b may carry a border element at its distal end, preferably an expandable border element, for instance a cage arrangement, preferably a cage arrangement comprising a membrane, see description of FIGS. 7 to 12, or an inflatable balloon, see description of FIGS. 17 and 18, or another appropriate border element that allows the isolation of a fluid transport volume TrV that is used for treatment of lung L or of another organ from a body fluid circuit, for instance the blood circuit BC. The border element on cannula L1b that is not shown in FIG. 1B isolates one end of the transport volume from the blood circuit of the subject. An introducer member and/or a sheath member may be used to enable and/or to ease the switching of the expandable border element from a non-expanded state that is used during insertion to an expanded state that is used for fixation of cannula L1b.

Further, single lumen cannula L2b may be inserted endovascular through the left internal jugular vein lIJV or the right internal jugular vein rIJV, i.e. through the internal jugular vein IJV that is not used by cannula L1b. Thereafter, cannula L2b is guided further through superior vena cava SVC, right atrium RA and then into left atrium LA. Cannula L2b may be inserted before or after cannula L1b. An introducer member and/or a guide wire may be used to enable and/or to ease insertion of cannula L2b. The maximum outer diameter of cannula L2b may be for instance 21 F or 23 F.

Single lumen cannula L2b may carry a further border element at its distal end, preferably an expandable border element, for instance a cage arrangement, preferably a cage arrangement comprising a membrane, see description of FIGS. 7 to 12, or an inflatable balloon, see description of FIGS. 17 and 18, or another appropriate border element that allows the isolation of a fluid transport volume TrV that is used for treatment of lung L or of another organ from a body fluid circuit, for instance the blood circuit BC. The border element (not shown in FIG. 1B) on cannula L2b isolates the other end of the transport volume TrV from the blood circuit BC of the subject. An introducer member and/or a sheath member may be used to enable and/or to ease the switching of the expandable border element from a non-expanded state that is used during insertion to an expanded state that is used for fixation of cannula L2b.

Pump Pb pumps the fluid flow from left atrium LA into to pulmonary artery PA. Thus the fluid flow that comprises a treatment substance flows within a transport volume TrV that comprises the pulmonary artery PA, tissue of lung L and the pulmonary veins PV. Pump Pb may be a roller pump or another pump that allows pulsatile fluid flow. Alternatively, continuous fluid flow may be used within circuitry CIRb.

Adsorber/filter unit ADSb may remove a treatment substance and/or other particles from the fluid flow in order to allow for instance correct adjustment of concentration by delivery unit C1b. Adsorber/filter unit ADSb may be arranged downstream from pump Pb or at another appropriate position within circuitry CIRb.

Treatment substance delivery unit C1b may insert drugs or other treatment substances, preferably downstream of adsorber/filter unit ADSb. The treatment substance may be a chemotherapy substance that is mentioned in the first part of the description and/or below.

Optional oxygenator OXYb and/or carbon dioxide (CO₂) removal unit may be used within circuitry CIRb to adjust the oxygen level within the fluid flow. The oxygenator OXYb and/or carbon dioxide (CO₂) removal unit and the adsorber/filter unit ADSb may be integrated into one unit.

Circuitry CIRb may be used for a lobe dedicated treatment of lung L. Alternatively, only parts of a lobe of lung L may be treated at one time. Lobe dedicated or partially treatment of lung L enables to use the natural function of the part of lung L that is not treated, i.e. for enriching the blood with oxygen and for removal of carbon dioxide. Thus, lung L assist may not be necessary. Even heart H assist may not be necessary as a blood flow through the untreated part of lung L is possible and afterload is reduced thereby. However, heart H assist and/or lung L assist may also be used if lung L is partially treated.

There are the following possibilities for partial treatment of lung L, for example:

Distal end of cannula L1b may be positioned in left pulmonary artery IPA and distal ends of cannula L2b may be positioned in both left pulmonary veins IPV. Cannula L2b may be a split tip cannula in this case, preferably carrying two expandable border elements, see FIG. 18 and corresponding description.

Distal end of cannula L1b may be arranged in right pulmonary artery rPA and distal ends of cannula L2b may be arranged in both right pulmonary veins rPV. Cannula L2b may be a split tip cannula in this case, preferably carrying two expandable border elements, see FIG. 18 and corresponding description.

Cannula L1b may be inserted farther into the right pulmonary artery rPA or into the left pulmonary artery IPA and/or cannula L2b may be inserted farther into both or one of the left pulmonary veins IPV or right pulmonary veins rPV.

It may be contemplated that also cannula L1b is a split tip cannula carrying two expandable border elements, for special medical applications.

Furthermore, the pumping direction may be directed reverse in each application scenario that is mentioned above, i.e. retrograde instead of antegrade. Furthermore, it is possible to switch direction of the fluid flow once or several times.

During treatment of the lung L it is possible that the patient inhales a medicament or treatment substance in order to promote the treatment by the substance or medicament that flows through the vessels of the lung L and through the tissue of the alveoli. The fluid flow within circuitry CIRb may comprise blood as a carrier substance. Alternatively other carrier substances may be used, for instance based on saline and/or on water.

FIG. 1C illustrates a further embodiment of a pIVLP (percutaneous in-vivo lung perfusion) circuitry CIRc that may be used for instance with medium fluid flow (for instance 1.5 liter per minute (L/min) to 3.5 liter per minute). Circuitry CIRc may comprise or consist of:

a dual lumen cannula DLc that may comprise a cannula L1c and a cannula L3c,

a single lumen cannula L2c,

an Y-connector,

a pump Pc,

an adsorber/filter unit ADSc,

a treatment substance delivery unit C1c, and

an optional oxygenator OXYc and/or carbon dioxide removal unit.

Instead of a dual lumen cannula DLc two separate single lumen cannulas L1c and L3c may be used as well. Dual lumen cannula DLc may be a fixed or a non-fixed dual lumen cannula. A fixed dual lumen cannula DLc does not allow separate insertion of the cannulas L1c, L3c which form the dual lumen cannula DLc. A non-fixed dual lumen cannula DLc allows the insertion of an outer cannula L3c without the insertion of an inner cannula L1c. After insertion of the outer cannula L3c the inner cannula L1c may be inserted separately. The advantage of the non-fixed dual lumen cannula DLc is less traumatic insertion, for instance due to a reduced stiffness during the insertion of the outer cannula L3c. A third type of dual lumen cannula DLc that may be used comprises cannula L1c and L3c arranged in parallel to each other and fixed to each other. Each of the cannulas L1c and L3c is arranged outside of the other cannula L3c or L1c in the last case.

A fixed dual lumen cannula DLc may be inserted endovascular through the right internal jugular vein rIJV or the left internal jugular vein lIJV, through superior vena cava SVC, right atrium RA, left ventricle LV and into pulmonary artery PA. An introducer member and/or a guide wire may be used to enable and/or to ease insertion. Thus, both cannulas L1c and L3c are in the right place if dual lumen cannula DLc is inserted. The distal end of the inner cannula L1c may extend up to the pulmonary artery PA. However, the distal end of outer cannula L3c may be arranged in right atrium RA thereby positioning at least one inlet hole in right atrium RA. Alternatively, it is possible to use an outer cannula that extends up to the right ventricle RV and comprises there at least one inlet hole. Additionally, at least one inlet hole may be arranged within the right atrium RA.

The maximum outer diameter of inner cannula L1c may be appropriately selected. The maximum outer diameter of cannula L3c may also be appropriately selected.

Independent of the type of cannula that is used, cannula L1c may carry a border element at its distal end, preferably an expandable border element, for instance a cage arrangement, preferably a cage arrangement comprising a membrane, see description of FIGS. 7 to 12, or an inflatable balloon, see description of FIGS. 17 and 18, or another appropriate border element that allows the isolation of a fluid transport volume TrV that is used for treatment of lung L or of another organ from a body fluid circuit, for instance the blood circuit BC. The border element (not shown in FIG. 1C) on cannula L1c may isolate one end of the transport volume TrV from the blood circuit BC of the subject. An introducer member and/or a sheath member may be used to enable and/or to ease the switching of the expandable border element from a non-expanded state that is used during insertion of cannula L1c to an expanded state that is used for fixation of cannula L1c. At least one optional expandable border element may be used on cannula L3c, e.g. within right atrium RA and/or right ventricle RV. If a cage arrangement is used, some of the wires may be omitted to allow the inner cannula to extend through the cage of the cage arrangement.

Examples of non-fixed dual lumen cannulas are described below with reference to FIGS. 3 to 6. A non-fixed dual lumen cannula DLc may be inserted as follows. The outer cannula L3c may be inserted endovascular through right internal jugular vein rIJV or through left internal jugular vein lIJV, through superior vena cava SVC up to right atrium RA or up to right ventricle RV. At least one optional expandable arrangement or two expandable arrangements may be used to fixate or to position the outer cannula L3c within right atrium RA and/or right ventricle RV. After the insertion of outer cannula L3c the inner cannula L1c is inserted into the outer cannula L3c until the inner cannula extends through the distal opening of outer cannula L3c and then further through left ventricle into pulmonary artery PA, where it is secured by the border element that comprises preferably an expandable arrangement. An introducer member and/or a guide wire may be used to enable and/or to ease insertion of the parts of the non-fixed dual lumen cannula DLc.

Furthermore, single lumen cannula L2c may be inserted endovascular through the left internal jugular vein rIJV or the right internal jugular vein lIJV, i.e. through the internal jugular vein IJV that is not used by cannula L1b. Cannula L2c may be inserted before or after cannula L1c and/or L3c. Thereafter, cannula L2c is guided further through superior vena cava SVC, right atrium RA into left atrium LA. An introducer member and/or a guide wire may be used to enable and/or to ease insertion of cannula L2c. The maximum outer diameter of cannula L2c may be for instance 21 F or 23 F.

Single lumen cannula L2c may carry a further border element at its distal end, preferably an expandable border element, for instance a cage arrangement, preferably a cage arrangement comprising a membrane, see description of FIGS. 7 to 12, or an inflatable balloon, see description of FIGS. 17 and 18, or another appropriate border element that allows the isolation of a fluid transport volume TrV that is used for treatment of lung L or of another organ from a body fluid circuit, for instance the blood circuit BC. The border element (not shown in FIG. 1C) on cannula L2c isolates the other end of the transport volume TrV from the blood circuit BC of the subject. An introducer member and/or a sheath member may be used to enable and/or to ease the switching of the expandable border element from a non-expanded state that is used during insertion to an expanded state that is used for fixation of cannula L2c.

The Y-connector may be used upstream of pump Pc in order to unite the flows that come from cannulas L2c and L3c. Further tubes, e.g. flexible tubes, may be used, to connect both cannulas L2c and L3c to pump Pc. The Y-connector may comprise three ports that are connected together by an inner lumen of the Y-connector.

Pump Pc may pump the fluid flow from left atrium LA and from right atrium RA and/or right ventricle RV into to pulmonary artery PA. Thus, the fluid flow that comprises a treatment substance flows within an isolated transport volume that comprises the pulmonary artery PA, tissue of the lung and the pulmonary veins PV. Pump Pc may be a centrifugal pump or another pump that allows pulsatile fluid flow. Alternatively, continuous fluid flow may be used.

Adsorber/filter unit ADSc may remove treatment substance and/or other particles from the fluid flow in order to allow correct adjustment of concentration by delivery unit C1b. Adsorber/filter unit ADSc may be arranged downstream from pump Pc or at another appropriate place within circuitry CIRc.

Treatment substance delivery unit C1c may insert drugs or other treatment substances, preferably downstream of adsorber/filter unit ADSc. The treatment substance may be a chemotherapy substance that is mentioned in the first part of the description or below.

Optional oxygenator OXYc and/or carbon dioxide (CO₂) removal unit may be used within circuitry CIRc to adjust the oxygen level within the fluid flow. The oxygenator OXYc and/or carbon dioxide (CO₂) removal unit and the adsorber/filter unit ADSc may be integrated into one unit.

Circuitry CIRc may be used for a lobe dedicated treatment of lung L. Alternatively, only parts of a lobe of lung L may be treated at one time. Lobe dedicated or partially treatment of lung L enables to use the natural function of the part of lung L that is not treated, i.e. for enriching the blood with oxygen and for removal of carbon dioxide. Thus, lung L assist may not be necessary. Even heart H assist may not be necessary as blood flow through the untreated part of lung L is possible and afterload is reduced thereby. However, heart H assist and/or lung L assist may also be used if lung L is partially treated.

There are the following possibilities for partial treatment of lung L, for example:

Distal end of cannula L1c may be positioned in left pulmonary artery IPA and distal ends of cannula L2c may be positioned in both left pulmonary veins IPV. Cannula L2c may be a split tip cannula in this case, preferably carrying two expandable border elements, see FIG. 18 and corresponding description.

Distal end of cannula L1c may be arranged in right pulmonary artery rPA and distal ends of cannula L2c may be arranged in both right pulmonary veins rPV. Cannula L2c may be a split tip cannula in this case, preferably carrying two expandable border elements, see FIG. 18 and corresponding description.

Cannula L1c may be inserted farther into the right pulmonary artery rPA or into the left pulmonary artery IPA and/or cannula L2c may be inserted farther into both or one of the left pulmonary veins IPV or right pulmonary veins rPV.

It may be contemplated that also cannula L1c is a split tip cannula carrying two expandable border elements, for special medical applications.

Furthermore, the pumping direction that is shown in FIG. 1C is antegrade. Retrograde flow through the lung may be established as well, for instance by changing the ports of circuitry CIRc to which cannula L1c and cannula L2c are connected. Furthermore, it is possible to switch the direction of the fluid flow once or several times.

In addition to the treatment of the lung L, especially of parts of lung L, right heart H assist may be realized by draining blood from right atrium RA and/or from right ventricle RV.

During treatment of the lung L it is possible that the patient inhales a medicament or treatment substance in order to promote the treatment by the substance or medicament that flows through the vessels of the lung L and through the tissue of the alveoli. The fluid flow within circuitry CIRc may comprise blood as a carrier substance. Alternatively other carrier substances may be used, for instance based on saline and/or on water.

FIG. 2A illustrates an extracorporeal retrograde lung perfusion circular blood flow circuitry 1006 comprising two dual lumen cannulas 1010 and 1040, two pumps P10a, P10b and an oxygenator device OXY10. Dual lumen cannula 1010 carries a cage arrangement 1016 near at least one inlet port that is arranged in pulmonary artery PA and an inlet portion 1090 that is arranged in right atrium RA. Second dual lumen cannula 1040 has at least one outlet port within ascending aorta aAO and an outlet portion 1084 in left atrium LA. The circuitry 1006 allows for instance the removal of thrombus from the lung L of patient. Alternatively, a chemotherapy of lung may be performed, a stem cell treatment or cleaning of the lung.

A fixed dual lumen cannula 1010 may be endovascularly inserted through right internal jugular vein IJV, superior vena cava SVC, right atrium RA, right ventricle RV, through pulmonary valve PVa into pulmonary artery PA. A guide wire (not shown) may be used to guide cannula 1010 to its final position. Alternatively, cannula 1010 may be inserted through the right subclavian vein and then along the same way as described above. Almost the whole blood that comes out of pulmonary artery PA is extracted into inner lumen of dual lumen cannula 1010, see arrow 1060, by using a membrane 1019 that is explained in more detail below. Other possibilities for insertion of a non-fixed dual lumen cannula 1010 will be explained below, e.g. first insertion of outer lumen and then insertion of inner lumen.

A fixed dual lumen cannula 1040 may be endovascularly inserted through left internal jugular vein IJV, superior vena cava SVC, right atrium RA, trans-septal, i.e. through the atrial septum AS between right atrium RA and left atrium LA, into left atrium LA. A guide wire (not shown) may be used to guide cannula 1040 to its final position. Alternatively, cannula 1040 may be inserted through the right subclavian vein. Almost the whole blood that exits the distal tip of the inner lumen of cannula 1040 is injected into ascending aorta aAO, see arrow 1070, by using a membrane 1049 that is explained in more detail below. Other possibilities for insertion of a non-fixed dual lumen cannula 1040 will be explained below, e.g. first insertion of outer lumen and then insertion of inner lumen.

An optional inlet tip 1014 may be mounted on distal end 1012 of cannula 1010. Inlet tip 1014 may comprise a plurality of inlet holes 1015 in its side wall. Additionally, there may be a hole within the distal end of inlet tip 1014. The sum of the cross section areas of the holes of tip 1014 may be greater than the inner cross section area of cannula 1010 at its distal end 1012, for instance greater than twice the area or the triple of the area. This means that blood can be removed even if one or more of inlet holes 1015 in inlet tip 1014 is or are clogged.

However, in other embodiments no inlet tip 1014 is used. Thus, there is only one inlet hole at distal end 1012 of cannula 1010, i.e. at the proximal end of the cage arrangement. This single inlet hole would be surrounded by cage arrangement 1016. A single inlet hole may allow higher flow rates compared to inlet tip 1014 that comprises lateral inlet holes.

Cage arrangement 1016 is one possible example. Other possible examples are described below with reference to FIGS. 7 to 12. Cage arrangement 1016 may comprise for instance between 6 to 12 flexible wires, beams or bars. There may be for instance 8 cage wires 1018 that span a sphere. The sphere may prevent that the side wall of left atrium LA covers one of inlet holes 1015 of tip 1014 or the single end-hole if no inlet tip 1014 is used. Furthermore, cage arrangement 1016 fixes distal end 1012 of cannula 910 to pulmonary artery PA. Thus it may not possible that cannula 1010 slides back through pulmonary valve PVa into left ventricle LV.

Membrane 1019 may cover only one half of cage arrangement 1016, e.g. a half that is between distal end 1012 of cannula 1010 and the mid of cage wires 1018. Examples of membranes that may be used on cage arrangement 1016 are described below with reference to FIG. 8.

Inlet portion 1090 may comprise a plurality of inlet holes that extend through the side wall of outer lumen of cannula 1010. Blood may be extracted by suction from right atrium RA into outer lumen of cannula 1010, preferably all blood or nearly all (for instance more than 90 percent of volume) blood that comes into right atrium RA, see arrow 1092. An optional cage arrangement may be arranged at inlet portion 1090.

Further to FIG. 2A, a tube 1020a may be connected to a proximal end of inner lumen of cannula 1010 and to an inlet of pump P10a. An outlet of pump P10a may be connected to a proximal end of outer lumen of cannula 1040 using a tube 1030a.

A tube 1020b may be connected to a proximal end of outer lumen of cannula 1010 and to an inlet of pump P10b. An outlet of pump P10b may be connected to oxygenator device OXY10. A tube 1030b is connected to an outlet of oxygenator device OXY10 and to the proximal end of inner lumen of cannula 1040. It is also possible to exchange the sequence of pump P10b and oxygenator device OXY10.

Pumps P10a, P10b may be peristaltic pumps, centrifugal pumps, membrane pumps or other kind of pumps. Oxygenator device OXY10 enriches blood with oxygen that comes out of right atrium RA and/or right ventricle RV (see inlet portion 1098 that is described in more detail below) and is then injected into ascending aorta aAO. Thus, the function of the lung is fulfilled by oxygenator device OXY10 during treatment of lung L and the right heart H is supported.

Tubes 1020a, 1020b, 1030a, 1030b may be made of a flexible material or of a more rigid material. The circuitry 1006 may further include one or more blood filter units or units for dialysis of blood.

Furthermore, a device may be used within circuitry 1006 for instance for injecting a drug or medicament and/or a treatment substance into the fluid flow of the part of circuitry 1006 that is driven by pump P10a. This fluid flow is driven through transport volume TRV within lung L of the patient.

Cannula 1040 may comprise an optional outlet tip 1050 that may have the same structure as inlet tip 1014 of cannula 1010. This means that outlet tip 1050 may comprise a plurality of outlet holes 1052 in its side wall and/or on its distal end. Additionally, cannula 1040 may have an optional cage arrangement 1046 on its distal end 1042. If there is no outlet tip 1050 a single end-hole may be used at the distal end of cannula 1040, i.e. at the proximal end of cage arrangement 1046.

Cage arrangement 1046 is one possible example. Other possible examples are described below with reference to FIGS. 7 to 12. Cage arrangement 1046 may comprise for instance between 6 to 12 flexible wires, beams or bars. There may be for instance 8 cage wires 1048 that span a sphere. The sphere and also the expelled blood prevent that the side wall of the ascending aorta aAO covers one of outlet holes 1052 of optional outlet tip 1050. The sphere prevents that the injected blood damages the walls of the aorta AO, i.e. the “sand blasting effect” is prevented or mitigated even if no outlet tip 1050 is used. Furthermore, cage arrangement 1046 fixes distal end 1042 of cannula 1040 within ascending aorta aAO.

Membrane 1049 may cover only one half of cage arrangement 1046, e.g. a half that is between the distal end of cannula 1040 and the mid of cage wires 1048. Examples of membranes that may be used on cage arrangement 1048 are described below with reference to FIG. 7.

Outlet portion 1084 of cannula 1040 may comprise a plurality of outlet holes 1085 that extend through the sidewall of the outer lumen of cannula 1040. Blood is expelled through outlet portion 1084 into the pairs of left and right pulmonary veins PV, see arrow 1075. Blood flow to left ventricle LV is thereby prevented by using membrane 1089. Outlet portion 1084 may be surrounded by an optional cage arrangement 1086.

Cage arrangement 1086 is one possible example. Other possible examples are described below with reference to FIGS. 7 to 12. Cage arrangement 1086 may comprise for instance between 6 to 12 flexible wires, beams or bars. There may be for instance 8 cage wires 1088 that span a sphere. The sphere and also the expelled blood may prevent that the side wall of left atrium LA covers one of outlet holes 1085. Moreover, the sand blasting effect is prevented or mitigated. Furthermore, cage arrangement 1086 fixes outlet portion 1086 of cannula 1040 within left atrium LA.

Membrane 1089 may cover only one half of cage arrangement 1086, e.g. a half that is defined by two cage wires 1088 that are arranged opposite to each other or nearly opposite. Examples of membranes that may be used on cage arrangement 1086 are described below with reference to FIG. 8.

In summary, the following blood or other fluid flows are established within circuitry 1006:
a) from pulmonary artery PA through inner lumen of cannula 1010 via pump P10a through outer lumen of cannula 1040 to left atrium LA, i.e. lung perfusion, and
b) from right atrium RA and/or right ventricle RV through outer lumen of cannula 1010 via pump P1Ob and OXY10 through inner lumen of cannula 1040 to ascending aorta aAO, i.e. external enrichment of blood with oxygen.

Flow a) is closed via transport volume TrV, i.e. via right and left pulmonary veins PV, tissue of lung L and right pulmonary artery rPA and left pulmonary artery IPA, and main pulmonary artery PA. Flow b) is closed via arteries of the body 100, for instance common femoral artery CFA, tissues of the body 100 and the veins of the body 100, for instance common femoral vein CFV.

No extra care has to be taken because both cannulas 1010 and 1040 are inserted into veins in which there is comparably low blood pressure compared to blood pressure in arteries.

The arrangement shown in FIG. 2A may be used for patients with lung L problems. Mobility of the patient is possible because no cannulas in femoral veins or femoral arteries are used. The arrangement shown in FIG. 2A may be named pIVLP (percutaneous in vivo lung perfusion) retrograde.

In other embodiments it is possible to insert cannula 1010 through internal jugular vein IJV/left subclavian vein to pulmonary artery PA as described above and cannula 1040 through right internal jugular vein IJV/right subclavian vein to ascending aorta aAO.

An optional inlet portion 1098 may be arranged on a part of the outer lumen of cannula 1010 that is within right ventricle RV if cannula 1010 is put in place. Thus, it is possible to extract more blood from the right side of heart H using inlet portions 1090 and 1098 during retrograde lung perfusion. An optional cage arrangement may be arranged around inlet portion 1098.

During treatment of lung L it is possible that the patient inhales a medicament or treatment substance in order to promote the treatment by the substance or medicament that flows through the vessels of the lung L and through the tissue of the alveoli. The same or different substances may be used for inhalation and perfusion.

The fluid flow within the part of circuitry 1006 which comprises pump P10a may comprise blood as a carrier substance. Alternatively other carrier substances may be used, for instance based on saline and/or on water. The treatment substance may be injected into the part of circuitry 1006 that comprises pump 10a. Furthermore, an adsorber/filter unit ADS and/or an oxygenator OXY and/or a carbon dioxide removal unit may be arranged within the part of circuitry 1006 that comprises pump 10a.

Furthermore, FIG. 2B illustrates an extracorporeal antegrade lung perfusion circular blood flow circuitry 1006 comprising two dual lumen cannulas 1010 and 1040, two pumps P10a, P10b and an oxygenator device OXY10. The proposed antegrade lung perfusion uses the arrangement 1006 of cannulas 1010 and 1040 as described above for retrograde lung perfusion. Reference is made to the description above in order to avoid unnecessary repetition. However, only the differences will be described. The main difference is that the direction of fluid flow that is generated in pump P10a is in the opposite direction now, see arrows 1094 and 1095. The direction of the blood flow or other fluid flow within the veins and arteries of lung L is now antegrade.

Arrow 1096 shows that fluid is expelled from distal end 1012 of cannula 1010 into pulmonary artery PA. Holes 1015 are outlet holes for antegrade lung perfusion and optional tip 1014 is an optional outlet tip antegrade lung perfusion. However, alternatively, a single end-hole may be used. Membrane 1014 directs fluid flow into pulmonary artery PA completely or almost completely. Furthermore, membrane 1014 may have a valve function allowing blood/fluid flow from right ventricle RV into pulmonary artery PA but not in the inverse direction.

Arrow 1097 shows that blood or other fluid that comes out of pulmonary veins PV is extracted by suction into outer lumen of cannula 1040. Holes 1085 are inlet holes for antegrade lung perfusion and portion 1084 is an inlet portion for antegrade lung L perfusion. However, a single end-hole may be used as well. Membrane 1089 directs fluid flow from pulmonary veins PV completely or almost completely into outer lumen of cannula 1040.

In summary, the following flows are established for antegrade lung perfusions within circuitry 1006:
a) from left atrium LA through outer lumen of cannula 1040 via pump P10a through inner lumen of cannula 1010 to pulmonary artery PA, i.e. lung perfusion, and
b) from right atrium RA and/or right ventricle RV through outer lumen of cannula 1010 via pump P10b and OXY10 through inner lumen of cannula 1040 to ascending aorta aAO, i.e. external enrichment of blood with oxygen.

Flow a) is closed via pulmonary artery PA, right pulmonary artery rPA/left pulmonary artery IPA, tissue of the lung L and right/left pulmonary veins PV. Flow b) is closed via arteries of the body, for instance common femoral artery CFA, tissues of the body and the veins of the body, for instance common femoral vein CFV.

Even for antegrade lung perfusion, no extra care has to be taken because both cannulas 1010 and 1040 are inserted into veins in which blood pressure is comparably low compared to blood pressure in arteries.

The arrangement shown in FIG. 2B may be used for patients with lung L problems. Mobility of the patient is possible because no cannulas in femoral veins or arteries are used. The arrangement for antegrade lung L perfusion shown in FIG. 2B may be named pIVLP (percutaneous in vivo lung perfusion) antegrade.

Also for antegrade lung L perfusion, it is possible to insert cannula 1010 through left internal jugular vein IJV or left subclavian vein to pulmonary artery PA as described above and cannula 1040 through right internal jugular vein IJV or right subclavian vein to ascending aorta aAO.

An alternative or optional inlet portion 1098 may be arranged on a part of the outer lumen of cannula 1010 that is within right ventricle RV if cannula 1010 is put in place. Thus, it is possible to extract more blood from the right side of heart H using inlet portions 1090 and 1098 during antegrade lung perfusion.

During treatment of lung L it is possible that the patient inhales a medicament or treatment substance in order to promote the treatment by the substance or medicament that flows through the vessels of lung L and through the tissue of the alveoli. Some or different substances may be used for inhalation and perfusion.

The fluid flow within the part of circuitry 1006 which comprises pump P10a may comprise blood as a carrier substance. Alternatively other carrier substances may be used, for instance based on saline and/or on water. The treatment substance may be injected into the part of circuitry 1006 that comprises pump 10a. Furthermore, an adsorber/filter unit ADS and/or an oxygenator OXY and/or a carbon dioxide removal unit may be arranged within the part of circuitry 1006 that comprises pump 10a.

Furthermore, it is possible to switch fluid flow direction between antegrade and retrograde, starting with antegrade fluid flow in lung L vessels or with retrograde fluid flow whichever is appropriate. Switching may be repeated during one treatment as often as necessary. Switching may ease the removal of at least one thrombus, especially of a blood thrombus.

Moreover, FIG. 2C illustrates an extra corporeal lobe dedicated antegrade lung perfusion circular blood flow circuitry 1006 comprising two dual lumen cannulas 1010 and 1040, two pumps P10a, P10b and an oxygenator device OXY10. The proposed lobe dedicated antegrade lung perfusion uses arrangement 1006 of cannulas 1010 and 1040 as described above for retrograde lung perfusion. Reference is made to the description above in order to avoid unnecessary repetition. However, only the differences will be described. One main difference is that the direction of blood/fluid flow generated by pump P10a is opposite to the direction mentioned above for FIG. 2A, see arrows 1094 and 1095. The other direction of fluid flow results in another direction of the blood flow or other fluid flow within the veins and arteries of lung L.

A further difference is that cannula 1010 would have a longer portion between inlet portion 1090 and distal end 1012 enabling an arrangement of a cage arrangement 1016a within left pulmonary artery IPA as shown in FIG. 2C. Cage arrangement 1016a is adapted to the diameter of left pulmonary artery IPA, i.e. it would be smaller than cage arrangement 1016. The other features of cage arrangement 1016a would be similar to the corresponding features of cage arrangement 1016 and would have an appropriate reduction in size, i.e. cage wires, membrane, optional outlet tip etc.

However, it is also possible to use an inflatable balloon Ba instead of cage arrangement 1016a, see description of FIGS. 17 and 18 below.

The membrane of cage arrangement 1016a may direct fluid flow that is expelled through inner lumen of cannula 1040 completely or almost completely into left pulmonary artery IPA. Furthermore, this membrane may have an optional valve function allowing blood flow from pulmonary artery PA also into left pulmonary artery IPA but not in the inverse direction.

Arrow 1097 shows that blood or other fluid that comes out of pulmonary veins PV is extracted by suction into outer lumen of cannula 1040. Holes 1085 are inlet holes for antegrade lung L perfusion and portion 1084 is an inlet portion for antegrade lung perfusion. Membrane 1089 may direct fluid flow from pulmonary veins PV completely or almost completely into outer lumen of cannula 1040. Right pulmonary veins rPV will expel the fluid flow that is injected by cage arrangement 1016a and left pulmonary veins IPV will expel normal blood flow.

In summary, the following flows are established for dedicated antegrade lung L perfusions within modified circuitry 1006:
a) from left atrium LA through outer lumen of cannula 1040 via pump P10a through inner lumen of cannula 1010 to pulmonary artery PA, i.e. lung perfusion, and
b) from right atrium RA and/or right ventricle RV through outer lumen of cannula 1010 via pump P10b and OXY10 through inner lumen of cannula 1040 to ascending aorta aAO, i.e. external enrichment of blood with oxygen.
c) normal blood flow from right ventricle RV through pulmonary artery PA, through right pulmonary artery rPA, tissue of lung L back via right pulmonary vein rPV into left atrium LA.

Flow a) is closed via the transport volume, i.e. via left pulmonary artery IPA, tissue of left lung lobe of lung L and left pulmonary veins IPV. Flow b) is closed via arteries of the body 100, for instance common femoral artery CFA, tissues of the body 100 and the veins of the body 100, for instance common femoral vein CFV.

Even for lobe dedicated antegrade lung L perfusion, no extra care has to be taken because both cannulas 1010 and 1040 are inserted into veins in which there is comparably low blood pressure compared to blood pressure within arteries.

The modified arrangement shown in FIG. 2C may be used for patients with lung problems. Mobility of the patient is possible because no cannulas in femoral veins or arteries are used. The arrangement for dedicated lobe antegrade lung perfusion shown in FIG. 2C may be named pIVLP (percutaneous in vivo lung perfusion) antegrade lobe dedicated.

Also for dedicated lobe antegrade lung perfusion, it is possible to insert cannula 1010 through left internal jugular vein IJV or left subclavian vein to pulmonary artery PA as described above and cannula 1040 through right internal jugular vein IJV or right subclavian vein to ascending aorta aAO.

In another embodiment the right lobe of lung L may be flushed in the same way as described above for the left lobe of lung L. In this case, cage arrangement 1016a of cannula 1010 having a longer portion between inlet portion 1090 and distal end 1012 than shown in FIG. 2C would be arranged within right pulmonary artery rPA. Left pulmonary artery IPA would be filled with normal blood flow coming from right ventricle RV.

Furthermore, treatment of both lobes of lung L is possible sequentially, e.g. treating the left lobe first and then the right lobe or vice versa. Several changes of the lobes that are treated are possible as well. The afterload that arises within heart H may be reduced in this way. Further positive effects may be possible as well. Detrimental effects may be limited to only one lobe. After a period of recreation the other lobe of lung L may be treated. Moreover, there may be diseases that require only the treatment of one lobe of lung L, for instance a thrombus in only one of the lobes of lung L.

An optional inlet portion 1098 may be arranged on a part of the outer lumen of cannula 1010 that is within right ventricle RV if cannula 1010 is put in place. Thus, it is possible to extract more blood from the right side of heart H using inlet portions 1090 and 1098 during dedicated lobe antegrade lung perfusion.

During dedicated treatment of only one lobe of the lung L it is possible that the patient inhales a further medicament or treatment substance in order to promote the treatment by the substance or medicament that flows through the vessels of the lung L and through the tissue of the alveoli. The further treatment substance may be the same as the treatment substance used for the transport volume TrV. Alternatively, substances may be used that are different from each other.

The fluid flow within the part of circuitry 1006 which comprises pump P10a may comprise blood as a carrier substance. Alternatively other carrier substances may be used, for instance based on saline and/or on water. The treatment substance may be injected into the part of circuitry 1006 that comprises pump 10a. Furthermore, an adsorber/filter unit ADS and/or an oxygenator OXY and/or a carbon dioxide removal unit may be arranged within the part of circuitry 1006 that comprises pump 10a.

A dedicated retrograde treatment of the lobes of lung L seems feasible if further measures are taken, for instance usage of at least one split tip cannula, for instance within the left pulmonary veins IPV or within the right pulmonary veins rPV, preferably comprising at least two border elements, preferably expandable border elements, see FIGS. 17 and 18.

FIG. 2D illustrates a further embodiment of a pIVLP (percutaneous in-vivo lung perfusion) circuitry CIRd that may be used for instance with high fluid flow and with blood oxygenation, for instance 3.5 liter per minute (L/min) to 5.0 liter per minute. Circuitry CIRd may comprise or consist of:

a dual lumen cannula DLd that may comprise a cannula L1d and a cannula L3d,

a single lumen cannula L2d,

a single lumen cannula L4d,

a first Y-pconnector,

a pump Pd,

an adsorber/filter unit ADSd,

a second Y-connector,

a treatment substance delivery unit C1d, and

an optional oxygenator OXYd and/or carbon dioxide removal unit.

Instead of a dual lumen cannula DLd two separate single lumen cannulas L1d and L3d may be used as well. Dual lumen cannula DLd may be a fixed or a non-fixed dual lumen cannula. A fixed dual lumen cannula DL2d does not allow separate insertion of the cannulas L1d, L3d which form the dual lumen cannula DL2d. A non-fixed dual lumen cannula DL2d allows the insertion of an outer cannula L3d without the insertion of an inner cannula L1d. After insertion of the outer cannula the inner cannula may be inserted separately. The advantage of the non-fixed dual lumen cannula DL2d is a less traumatic insertion, for instance due to a reduced stiffness during the insertion of the outer cannula L3d. A third type of dual lumen cannula DL2d that may be used comprises cannula L1d and L3d arranged in parallel to each other and fixed to each other. Each of the cannulas L1d and L3d is arranged outside of the other cannula L3d or L1d in the latter case.

A fixed dual lumen cannula DLd may be inserted in the same way as fixed dual lumen cannula DLc, see description above.

The maximum outer diameter of inner cannula L1d may be selected appropriately. The maximum outer diameter of cannula L3d may be appropriately selected

Independent of the type of cannula that is used, cannula L1d may carry a border element at its distal end, preferably an expandable border element, for instance a cage arrangement, preferably a cage arrangement comprising a membrane, see description of FIGS. 7 to 12, or an inflatable balloon Ba, see description of FIGS. 17 and 18, or another appropriate border element that allows the isolation of a fluid transport volume TrV that is used for treatment of lung L or of another organ from a body fluid circuit, for instance the blood circuit BC. The border element (not shown in FIG. 2D) on cannula L1d may isolate one end of the transport volume TrV from the blood circuit BC of the subject. An introducer member and/or a sheath member may be used to enable and/or to ease the switching of the expandable border element from a non-expanded state that is used during insertion to an expanded state that is used for fixation of the cannula L1d. At least one optional expandable border element may be used on cannula L3d, e.g. within right atrium RA and/or right ventricle RV. If a cage arrangement is used on cannula L3d, some of the wires may be omitted to allow the inner cannula to extend through the cage.

Examples of non-fixed dual lumen cannulas DLd are described below with reference to FIGS. 3 to 6. A non-fixed dual lumen cannula DLd may be inserted as described above for non-fixed dual lumen cannula DLc.

Furthermore, single lumen cannula L2d may be inserted endovascular as described above for single lumen cannula L2c. The maximum outer diameter of cannula L2d may be for instance 21 F or 23 F.

Single lumen cannula L2d may carry a further border element at its distal end, preferably an expandable border element, for instance a cage arrangement, preferably a cage arrangement comprising a membrane, see description of FIGS. 7 to 12, or an inflatable balloon Ba, see description of FIGS. 17 and 18, or another appropriate border element that allows the isolation of a fluid transport volume TrV that is used for treatment of lung L or of another organ from a body fluid circuit, for instance the blood circuit BC. The border element (not shown in FIG. 2D) on cannula L2d may isolate the other end of the transport volume TrV from the blood circuit BC of the subject. An introducer member and/or a sheath member may be used to enable and/or to ease the switching of the expandable border element from a non-expanded state that is used during insertion to an expanded state that is used for fixation of cannula L2d.

Single lumen cannula L4d may be inserted endovascular into an artery of the body 100 of the subject, for instance into the left femoral artery or into the right femoral artery. However, a transcaval access may be used, for instance from common femoral vein CFV to common femoral artery CFA.

The first Y-connector may be used upstream of pump Pd in order to unite the flows that come from cannulas L2d and L3d. Further tubes, e.g. flexible tubes, may be used, to connect both cannulas L2d and L3d to pump Pd. The first Y-connector may comprise three ports that are fluidically connected together by an inner lumen of the first Y-connector.

Pump Pd may pump the fluid flow from left atrium LA and the blood flow from right atrium RA and/or right ventricle RV into pulmonary artery PA and into an artery, preferably femoral. Thus, the fluid flow that comprises a treatment substance flows within an isolated transport volume TrV that comprises the pulmonary artery PA, tissue of lung L and the pulmonary veins PV. Pump Pd may be a centrifugal pump or another pump that allows pulsatile fluid flow. Alternatively, continuous fluid flow may be used.

Adsorber/filter unit ADSd may remove treatment substance and/or other particles from the fluid flow in order to allow correct adjustment of concentration by delivery unit C1d. Adsorber/filter unit ADSd may be arranged downstream from pump Pd or at another appropriate place within circuit CIRd.

Second Y-connector may comprise three ports that are fluidically connected together by an inner lumen of the first Y-connector. A first port of the second Y-connector may be connected to an output port of adsorber/filter unit ADSd. A second port of the second Y-connector may be connected to cannula L1d. A third port of the second Y-connector may be connected to single lumen cannula L4d,

Treatment substance delivery unit C1d may insert drugs or other treatment substances, preferably downstream of adsorber/filter unit ADSc and upstream of cannula L1d. The treatment substance may be a chemotherapy substance that is mentioned in the first part of the description.

Optional oxygenator OXYd and/or carbon dioxide (CO₂) removal unit may be used within circuitry CIRd to adjust the oxygen level within the fluid flow and/or within the blood circuit BC. The oxygenator OXYd and/or carbon dioxide (CO₂) removal unit and the adsorber/filter unit ADSd may be integrated into one unit. Oxygenator OXYd may be arranged between pump Pd and adsorber/filter unit ADSd or at another appropriate position within circuitry CIRd.

Circuitry CIRd may be used for a lobe dedicated treatment of lung L. Alternatively, only parts of a lobe of lung L may be treated at one time. Lobe dedicated or partially treatment of lung L enables to use the natural function of that part of the lung L that is not treated, i.e. for enriching the blood with oxygen and for removal of carbon dioxide. Thus, lung L assist may not be necessary. Even heart H assist may not be necessary as blood flow through the untreated part of lung L is possible and afterload is reduced thereby. However, heart H assist and/or lung L assist may also be used if lung L is partially treated.

Because heart H assist and lung L support are realized by circuitry CIRd it is also possible to treat both lobes of the lung at the same time, i.e. simultaneously.

There are the following possibilities for partial treatment of lung L, for example:

Distal end of cannula L1d may be positioned in left pulmonary artery IPA and distal ends of cannula L2d may be positioned in both left pulmonary veins IPV. Cannula L2d may be a split tip cannula in this case, preferably carrying two expandable border elements, see FIG. 18 and corresponding description.

Distal end of cannula L1d may be arranged in right pulmonary artery rPA and distal ends of cannula L2d may be arranged in both right pulmonary veins rPV. Cannula L2d may be a split tip cannula in this case, preferably carrying two expandable border elements, see FIG. 18 and corresponding description.

Cannula L1d may be inserted farther into the right pulmonary artery rPA or into the left pulmonary artery IPA and/or cannula L2d may be inserted farther into both or one of the left pulmonary veins IPV or right pulmonary veins rPV.

It may be contemplated that also cannula L1d is a split tip cannula carrying two expandable border elements, for special medical applications.

Furthermore, the pumping direction is antegrade for the example that is shown in FIG. 2D. Retrograde flow through the lung may be established as well, for instance by changing the ports of circuitry CIRd to which cannula L1d and cannula L2d are connected. Furthermore, it is possible to switch direction of the fluid flow once or several times.

In addition to the treatment of lung L, especially of parts of lung L, right heart H assist may be realized by draining blood from right atrium RA and/or from right ventricle RV.

The part of lung L that is not treated may fulfill its normal function, i.e. enriching the blood with oxygen and removing carbon dioxide. Furthermore, lung L assist may be realized via the oxygenated blood that is delivered by cannula L4d.

Assuming for example that the flow rate of the overall fluid within circuitry CIRd is 5 liters there may be the following flow rates:

for instance 3 liters per minute (L/min) within cannula L2d,

for instance 2 liters per minute within cannula L3d,

for instance 5 liters per minute at an input port of adsorber/filter unit ADSd.

This would result in a flow rate of about 3 liters within cannula L1d and in a flow of about 2 liters per minute within cannula L4d. However, other proportions of blood may be adjusted, for instance using other outer diameters of the cannulas L1d to L4d resulting also into other inner diameters.

During treatment of lung L it is possible that the patient inhales via the trachea a medicament or treatment substance in order to promote the treatment by the substance or medicament that flows within the fluid flow through the vessels of the lung L and through the tissue of the alveoli. Both substances may be the same substance. Alternatively, different treatment substances may be used within the fluid flow and for inhalation.

The fluid flow within circuitry CIRd may comprise blood as a carrier substance. Alternatively other carrier substances may be used, for instance based on saline and/or on water.

FIG. 2E illustrates a further embodiment of a pIVLP (percutaneous in-vivo lung perfusion) circuitry CIRe that may be used with for instance with high double fluid flow and optional inhalation. The fluid flow rate may be in the range of 5 liters per minute to 8 liters per minute. Circuitry CIRe may comprise or consist of:

a dual lumen cannula DLe that may comprise a cannula L1e and a cannula L3e,

a single lumen cannula L2e,

a single lumen cannula L2e,

a first pump Pe1,

a second pump Pe2,

an adsorber/filter unit ADSe,

a treatment substance delivery unit C1e, and

an optional oxygenator OXYe and/or carbon dioxide removal unit.

Instead of a dual lumen cannula DLe two separate single lumen cannulas L1e and L3e may be used as well. Dual lumen cannula DLe may be a fixed or a non-fixed dual lumen cannula. A fixed dual lumen cannula DLe does not allow separate insertion of the cannulas L1e, L3e which form the dual lumen cannula DLe. A non-fixed dual lumen cannula DLe allows the insertion of an outer cannula L3e without the insertion of an inner cannula L1e. After insertion of the outer cannula L3e the inner cannula L1e may be inserted separately. The advantage of the non-fixed dual lumen cannula DLe is a more atraumatic insertion, for instance due to a reduced stiffness during the insertion of the outer cannula L3e. A third type of dual lumen cannula DLe that may be used comprises cannula L1e and L3e arranged in parallel to each other and fixed to each other. Each of the cannulas L1e and L3e may be arranged outside of the other cannula L3e or L1e in this third type of dual lumen cannula DLe.

A fixed dual lumen cannula DLe may be inserted in the same way as fixed dual lumen cannula DLc, see description above.

The maximum outer diameter of inner cannula L1e may be for instance 21 F or less. The maximum outer diameter of cannula L3e may be for instance 29 F or less.

Independent of the type of cannula that is used, cannula L1e may carry a border element at its distal end, preferably an expandable border element, for instance a cage arrangement, preferably a cage arrangement comprising a membrane, see description of FIGS. 7 to 12, or an inflatable balloon Ba, see description of FIGS. 17 and 18, or another appropriate border element that allows the isolation of a fluid transport volume TrV that is used for treatment of lung L or of another organ from a body fluid circuit, for instance from the blood circuit BC. The border element on cannula L1e that is not shown in FIG. 2E may isolate one end of the transport volume TrV from the blood circuit BC of the subject. An introducer member and/or a sheath member may be used to enable and/or to ease the switching of the expandable border element from a non-expanded state that is used during insertion to an expanded state that is used for fixation of the cannula L1e. At least one optional expandable border element may be used on cannula L3e, e.g. within right atrium RA and/or right ventricle RV. If a cage arrangement is used, some of the wires may be omitted to allow the inner cannula L1e to extend through the cage of the cage arrangement.

Examples of non-fixed dual lumen cannulas are described below with reference to FIGS. 3 to 6. A non-fixed dual lumen cannula DLe may be inserted as described above for non-fixed dual lumen cannula DLc.

Furthermore, single lumen cannula L2e may be inserted endovascularly as described above for single lumen cannula L2c. The maximum outer diameter of cannula L2e may be for instance 21 F or 23 F or more.

Single lumen cannula L2e may carry a further border element at its distal end, preferably an expandable border element, for instance a cage arrangement, preferably a cage arrangement comprising a membrane, see description of FIGS. 7 to 12, or an inflatable balloon Ba, see description of FIGS. 17 and 18, or another appropriate border element that allows the isolation of a fluid transport volume TrV that is used for treatment of lung L or of another organ from a body fluid circuit, for instance the blood circuit BC. The border element (not shown in FIG. 2E) on cannula L2e may isolate the other end of the transport volume TrV from the blood circuit BC of the subject. An introducer member and/or a sheath member may be used to enable and/or to ease the switching of the expandable border element from a non-expanded state that is used during insertion to an expanded state that is used for fixation of cannula L2e.

Single lumen cannula L2e may be inserted endovascular into an artery of the body 100 of the subject, for instance into the left femoral artery or into the right femoral artery. However, a transcaval access may be used, for instance from common femoral vein CFV to common femoral artery CFA.

Pump Pe1 may be a centrifugal pump or another pump that allows pulsatile fluid flow. Alternatively, continuous fluid flow may be used. An input port of pump Pe1 may be connected to the proximal end of cannula L2e. An output port of pump Pe1 may be connected to an input of adsorber/filter unit ADSe. An output port of adsorber/filter unit ADSe may be connected to the proximal end of cannula L1e and to treatment substance delivery unit C1e.

Pump Pe1 may pump the fluid flow from left atrium LA into to pulmonary artery PA. Thus, the fluid flow that comprises a treatment substance flows within an isolated transport volume TrV that comprises pulmonary artery PA, tissue of lung L and pulmonary veins PV.

Pump Pe2 may be a centrifugal pump or another pump that allows pulsatile fluid flow. Alternatively, continuous fluid flow may be used. An input port of pump Pe2 may be connected to the proximal end of cannula L3e. An output port of pump Pe2 may be connected to an input of oxygenator OXYe and/or carbon dioxide removal unit. An output port of oxygenator OXYe and/or carbon dioxide removal unit may be connected to the proximal end of cannula L2e

Pump Pe2 may pump body fluid, especially blood from right atrium RA and/or right ventricle RV into an artery, preferably femoral. This blood may be oxygenated by oxygenator OXYe.

Adsorber/filter unit ADSe may remove treatment substance and/or other particles from the fluid flow in order to allow correct adjustment of concentration by delivery unit C1e. Adsorber/filter unit ADSe may be arranged downstream from pump Pe1 or on another appropriate place within circuitry CIRe.

Treatment substance delivery unit C1e may insert drugs or other treatment substances, preferably downstream of adsorber/filter unit ADSc and upstream of cannula L1e. The treatment substance may be a chemotherapeutic substance that is mentioned in the first part of the description.

Oxygenator OXYe and/or carbon dioxide (CO₂) removal unit may be used within circuitry CIRe to adjust the oxygen level within the blood flow.

Circuitry CIRe may be used for a lobe dedicated treatment of lung L. Alternatively, only parts of a lobe of lung L may be treated at one time. Lobe dedicated or partially treatment of lung L enables to use the natural function of that part of lung L that is not treated, i.e. for enriching the blood with oxygen and/or for removal of carbon dioxide. Thus, lung L assist may not be necessary. Even heart H assist may not be necessary as blood flow through the untreated part of lung L is possible and afterload to heart H is reduced thereby. However, heart H assist and/or lung L assist may also be used if lung L is partially treated.

Because heart H assist and lung L support are realized by circuitry CIRe it is also possible to treat both lobes of lung L at the same time, i.e. simultaneously.

There are the following possibilities for partial treatment of lung L, for example:

Distal end of cannula L1e may be positioned in left pulmonary artery IPA and distal ends of cannula L2e may be positioned in both left pulmonary veins IPV. Cannula L2e may be a split tip cannula in this case, preferably carrying two expandable border elements, see FIG. 18 and corresponding description.

Distal end of cannula L1e may be arranged in right pulmonary artery rPA and distal ends of cannula L2e may be arranged in both right pulmonary veins rPV. Cannula L2e may be a split tip cannula in this case, preferably carrying two expandable border elements, see FIG. 18 and corresponding description.

Cannula L1e may be inserted farther into the right pulmonary artery rPA or into the left pulmonary artery IPA and/or cannula L2e may be inserted farther into both or one of the left pulmonary veins IPV or right pulmonary veins rPV.

It may be contemplated that also cannula L1e is a split tip cannula carrying two expandable border elements, for instance for special medical applications.

Furthermore, the pumping direction in the example that is shown in FIG. 2E is antegrade. Retrograde flow through lung L may be established as well, for instance by changing the ports of circuitry CIRe to which cannula L1e and cannula L2e are connected. Alternatively, it is possible to change the pumping direction of pump Pe1 and exchange the place of unit C1e and adsorber/filter unit ADSe. Furthermore, it is possible to switch direction of the fluid flow once or several times.

In addition to the treatment of lung L, especially of parts of lung L, right heart H assist is realized by draining blood from right atrium RA and/or from right ventricle RV.

The part of lung L that is not treated may fulfill its normal function, i.e. enriching the blood with oxygen and/or removing carbon dioxide. Furthermore, lung L assist may be realized via the oxygenated blood that is delivered by cannula L2e.

The flow rate that is generated by pump P1e may be adjusted independently of the flow rate that is generated by pump P2e. Thus, a wider range of medical application scenarios is possible. Adjustments may be made before and/or during treatment. A further technical effect of the embodiment that is shown in FIG. 2E is the strict separation between both liquid flows, i.e. the fluid flow that is used for treatment of lung L and that is driven by pump Pe1 and the oxygenated blood flow that is driven by pump Pe2.

During treatment of lung L it is possible that the patient inhales via the trachea a medicament or treatment substance in order to promote the treatment by the substance or medicament that flows within the fluid flow through the vessels of the lung L and through the tissue of the alveoli. The two treatment substances may be the same substances or may be different from each other. An inhalation device INH may be used that is coupled to a treatment substance delivery unit C2.

The fluid flow within circuitry CIRe may comprise blood as a carrier substance. Alternatively, other carrier substances may be used, for instance based on saline and/or on water, see description above and below.

Furthermore, only one lobe of the lung L may be treated. Alternatively, several parts/lobes of lung L may be treated in sequence. There may be only one change between the treatments of two different parts, for instance lobes, or there may be more than one change between these parts. This is valid also for the other embodiments, for instance the embodiments shown in FIG. 1B, 1C and 2D. These changes of parts may be combined with changes of the flow directions.

A further oxygenator and/or carbon dioxide removal unit may be included in the part of circuitry CIRe that is driven by pump Pe1 in order to adjust the oxygen level within the fluid flow that flows through the transport volume TrV within the lung L.

Other applications of the proposed cage arrangements and/or dual lumen cannulas than these shown in FIGS. 1A to 2E are possible as well. All cannulas that are shown may be used with or without cages or balloons BA.

The cannula systems CS1 to CS3 that are described below with reference to FIGS. 3 to 5 are further examples for dual lumen cannula systems shown in FIG. 1A to 2E. FIG. 3 illustrates a cannula system CS1 having an inner cannula I1 and an outer cannula O1 that are arranged coaxially relative to each other with the inner cannula I1 arranged inside the outer cannula O1. Outer cannula O1 may be named as a first cannula in the claims. Inner cannula I1 may be named as a second cannula in the claims.

Outer cannula O1 is inserted into body 100 first, i.e. preferably before the inner cannula I1 will be inserted. Only after the insertion of the outer cannula O1 into body 100, preferably after the insertion of outer cannula O1 is completed, i.e. the outer cannula O1 has reached its destination position, inner cannula I1 is inserted into outer cannula O1 and then further beyond the distal end of outer cannula O1.

Both cannulas O1 and I1 are bendable up to a specific degree, i.e. they are bendable in radial directions. However, the diameter of cannulas O1 and I1 may not be variable in the sense that the area of the diameter cross section may be increased or decreased essentially.

Outer cannula O1 may have a circular or oval cross section along its entire length. A port P1a of outer cannula O1 may be arranged at a proximal P end of a sidewall of outer cannula O1. The proximal P end of outer cannula O1 may comprise a proximal surface, for instance a flat surface, that may have an opening OP1. Opening OP1 may be arranged on the longitudinal axis A of outer cannula O1.

Inner cannula I1 may also have a circular or oval cross section along its entire length. A port P1b of inner cannula I1 may be arranged at a proximal P end of inner cannula I1. Inner cannula I1 may be inserted through opening OP1 into outer cannula O1. Thereby, the inner cannula I1 may be arranged on the longitudinal axis A of outer cannula O1. At least one mounting portion MP1 or mounting elements may be arranged on an outer surface of inner cannula I1, e.g. protruding radially outward, and/or on an inner surface of outer cannula O1, e.g. protruding radially inward. Mounting portions MP1 may center inner cannula I1 within outer cannula O1.

A sealing element S1 may be used to seal cannula system CS1 proximally. Sealing element S1 may be arranged within opening OP1 or at another appropriate location. Sealing element S1 may be an O-ring in the simplest case. Alternatively, a multi-flap valve or a membrane may be used.

An optional fixation element FE1 may be arranged completely outside of outer cannula O1. Fixation element FE1 may have a first state in which axial movement M1 of inner cannula I1 relative to outer cannula O1 is possible or allowed and a second state that blocks such axial movement. Fixation element FE1 may operate automatically or semi-automatically or may be operated manually. Thus, fixation element FE1 may block axial movement if a predetermined length of inner cannula I1 is introduced into outer cannula O2. Alternatively, blocking may be performed manually at several positions of inner cannula I1 within outer cannula O1. It may be possible to bring fixation element FE1 back to the first state after it is in the blocking state.

Alternatively, fixation element FE1 may be arranged partly or completely within outer cannula O1. If it is completely within outer cannula O1 manual access to fixation element FE1 may be possible by operating elements. Alternatively, no manual access may be possible, i.e. fixation element FE1 may be operated in an automatic or semi-automatic mode depending for instance on the overlapping length of both cannulas I1 and O1.

FIG. 4 illustrates a cannula system CS2 having an inner (second) cannula I2 that is arranged loosely within an outer (first) cannula O2. Outer cannula O2 may be named as a first cannula in the claims. Inner cannula I2 may be named as a second cannula in the claims.

Outer cannula O2 is inserted into body 100 first, i.e. preferably before the inner cannula I2 will be inserted. Only after the insertion of the outer cannula O2 into body 100, preferably after the insertion of outer cannula O2 is completed, i.e. the outer cannula O2 has reached its destination position, inner cannula I2 is inserted into outer cannula O2 and then further beyond the distal end of outer cannula O2.

Both cannulas O2 and I2 are bendable up to a specific degree, i.e. they are bendable in radial directions. However, the diameter of cannulas O2 and I2 may not be variable in the sense that the area of the diameter cross section may be increased or decreased essentially.

Outer cannula O2 may have a circular or oval cross section along its entire length. A port P2a of outer cannula O2 may be arranged at a proximal P end of outer cannula O2 that may be arranged on the longitudinal axis of outer cannula O2. The sidewall of outer cannula O2 may have an opening OP2 at its proximal P end. Opening OP2 may face laterally and or transversally relative to longitudinal axis A of outer cannula O2.

Inner cannula I2 may also have a circular or oval cross section along its entire length. A port P2b of inner cannula I2 may be arranged at a proximal P end of inner cannula I1. Inner cannula I2 may be inserted through opening OP2 into outer cannula O2. Thereby, the inner cannula I1 may be arranged loosely radially to longitudinal axis A of outer cannula O1. A mounting portion may not be necessary.

A sealing element S2 may be used to seal cannula system CS2 proximally. Sealing element S2 may be arranged within opening OP2 or at another appropriate location. Sealing element S2 may be an O-ring in the simplest case. Alternatively, a multi-flap valve or a membrane may be used.

An optional fixation element FE2 may be arranged completely outside of outer cannula O2. Fixation element FE2 may have a first state in which an axial movement M2 of inner cannula I2 relative to outer cannula O2 is possible or allowed and a second state that blocks such axial movement. Fixation element FE2 may operate automatically or semi-automatically or may be operated manually. Thus fixation element FE2 may block axial movement if a predetermined length of inner cannula I2 is introduced or inserted into outer cannula O2. Alternatively, blocking may be performed manually at several positions of inner cannula I2 within outer cannula O2. It may be possible to bring fixation element FE2 back to the first state after it is in the blocking state.

Alternatively, fixation element FE2 may be arranged partly or completely within outer cannula O2. If it is completely within outer cannula O2 manual access to fixation element FE2 may be possible by operating elements. Alternatively, no manual access may be possible, i.e. fixation element FE2 may be operated in an automatic or semi-automatic mode depending for instance on the overlapping length of both cannulas I2 and O2.

FIG. 5 illustrates a cross section of another cannula system CS3 that comprises an outer cannula O3 and an inner cannula I3. Outer cannula O3 may be named as a first cannula in the claims. Inner cannula I3 may be named as a second cannula in the claims.

Outer cannula O3 has a circular inner cross section, preferably along its whole length. Alternatively, outer cannula O3 may have an oval or elliptic inner cross section, preferably along its whole length.

Inner cannula I3 has an outer cross section that is complementary to the inner cross section of outer cannula O3 and that leaves a lumen (first lumen in the claims) for the transport a fluid through outer cannula O3. If outer cannula O3 has an oval inner cross section, the outer cross section of inner cannula may also be oval or elliptic minus a part that is used for fluid transport in outer cannula O3.

The fluid may be blood or may comprise blood, for instance blood enhanced with a medicament or drug. Alternatively other fluids than blood may be used.

Inner cannula I3 may have a flat outer surface that is arranged for instance along the longitudinal axis A of outer cannula O3. Alternatively, this flat surface of inner cannula I3 may be arranged on a side of the longitudinal axis A of outer cannula O3 on which the first lumen of the outer cannula O3 for fluid transport is located, see line L3. In a further alternative, the flat surface of inner cannula I3 may be arranged on a side of the longitudinal axis A of outer cannula O3 that is opposite to the side that comprises the main part of the first lumen of the outer cannula O3 for fluid transport, see line L4.

No mounting elements may be necessary in cannula system CS3. However, it is possible to use mounting elements that position or fix the inner cannula I3 radially relative to outer cannula O3. Positioning would be easier than in cannula system CS1 because the complementary shapes of inner cannula I3 and outer cannula O3 may be used to enhance a specific positioning of inner cannula I3 within outer cannula O3.

FIG. 6 illustrates an embodiment of a dual lumen cannula system 1500 comprising at least one pre-bended outer cannula 1508 and an inner cannula that is not shown. Cannula system 1500 is adapted for a stepwise insertion of the cannula 1508 and of the other cannula, i.e. first outer cannula 1508 and then inner cannula. Dual lumen system 1500 may comprise:

a locking mechanism 1502 that may lock an introducer that is used for introducing outer cannula 1508. FIG. 12 shows an example in which an introducer is inserted into a cannula system. The introducer may be locked by force fitting or by another appropriate mechanism.

an adapter portion 1504 that may be used to connect locking mechanism removably to cannula system 1500,

a handle portion 1506 that may also be connected removably to cannula system 1500 and that is used to ease introducing of outer cannula 1508 or inner cannula into body 100 of a patient. Handle portion 1506 may be compressible by a medical clamp or forceps.

There may be a pre-bended kink K or bend that is between a long straight portion of cannula 1508 of system 1500 and a shorter straight portion. Cannula 1508 may have a straight insertable length L10 up to kink K and a short straight portion of cannula system 1500 having an insertable length L20 between kink K and the distal end. Kink K may also be positioned on other positions than the position shown in FIG. 6, e.g. more distally D or more proximally P. Cannula system 1500, especially cannula 1508, is or may be flexible, i.e. it is possible to bend each portion and of course to bring the whole cannula system 1500 in a straight shape. However, without external forces, kink K will bring cannula 1508 of system 1500 in the shape that is shown in FIG. 6 again. The inner cannula of cannula system 1500 may not have a kink K but may be straight. Alternatively, the inner cannula of cannula system 1500 may have a kink K

Although, cannula 1508 is shown having no diameter variable arrangement DVA or cage arrangement on the distal end there may be such a diameter variable arrangement DVA1. Cannula 1508 may comprise only one end hole that may be closed by a closure element that allows passage of the inner cannula but not of blood into cannula 1508 through the end hole or vice versa. Insertion of the inner cannula may further be promoted if at least one wire of diameter variable arrangement DVA1 is omitted. The lateral or side holes of cannula 1508 may be placed within the right atrium RA of the heart H whereas the cage arrangement DVA1 may be placed within the left atrium LA of the heart H.

In an alternative embodiment a cage arrangement or diameter variable arrangement DVA2 is used and the distal tip with lateral holes is omitted. There may be a membrane connected to diameter variable arrangement DVA2 that has an opening which faces laterally, see cage arrangement 1086 in FIGS. 2A, 2B and 2C. Cannula 1508 may have only one end-hole in this case, preferably an end-hole that is not closed by a valve and/or membrane. Introduction of the inner cannula of cannula system 1500 may be easier compared to the case in which a distal tip having lateral holes is include within diameter variable arrangement DVA2. However, in a further alternative, a distal tip with lateral holes and/or with an end hole may be used. Insertion of the inner cannula may further be promoted if at least one wire of diameter variable arrangement DVA2 is omitted.

Cannula 1508 may be used as a delivery cannula or as a drainage cannula.

Cannula system 1500 may be used for jugular access to heart H or for other purposes. Length L10 refers to the length from the proximal end of handle portion 1506 to the pre bended kink K, i.e. the length of the longer straight portion of cannula 1508. An example for length L10 is 300 mm Other values for length L10 are also possible.

Length L20 is the length of the pre bended distal portion, i.e. measured from the pre bended kink K to the distal end of cannula 1508, and without the length of a diameter variable arrangement if present. An example for length L20 may be for instance 70 mm (millimeter). Other values for length L20 are possible as well. Preferred values for length L20 are within the range of 3 cm to 7 cm.

An angle W1 between the two straight portions of cannula system 1500 at kink K may have a value of 130 degrees. However, a value within the range of 70 degrees to 145 degrees is also possible.

Cannula system 1500 may be used for left or right jugular or for left or right subclavian access to heart H or for other purposes. Longer cannulas are necessary for left side access and or for femoral access to the heart H. Modifications may be made with regard to length L10 and or length L20. Furthermore, the kink K may be at another position and angle W1 may have another value. It may also be useful to have a second pre-bended kink

For cannula 1508 of cannula system 1500 the following table may be valid:

| size of cannula in F/ | | | |
|---|---|---|---|
| overall length in cm | SL1 in mm | SL2 in mm | L10 + L20 in cm |
| 21 F/32 | 7.0 | 2.3 | 32 |
| 21 F/42 | 7.0 | 2.3 | 42 |
| 21 F/62 | 7.0 | 2.3 | 62 |
| 21 F/72 | 7.0 | 2.3 | 72 |
| 31 F/42 | 10.3 | 2.3 | 42 |

There may be further intermediate sizes of cannulas having for instance an outer diameter of 23 F (French), 25 F, 27 F and 29 F combined with an overall length L10 plus L20 of for instance 32 cm, 42 cm or 62 cm. The overall length L10 plus L20 is the insertable length of cannula 1408.

SL1 is the outer diameter of outer or first cannula 1508 in French. SL2 is the diameter of lateral holes in the distal tip. The numbers given in the table or given above may vary within a range of minus 10 percent to plus 10 percent. Other sizes of the cannula system 1500 are possible as well.

Furthermore, a cage arrangement (diameter variable arrangement DVA) may be mounted on distal end of inner cannula of cannula system 1500. Additionally or alternatively, a cage arrangement (diameter variable arrangement DVA) may be mounted on distal end of outer cannula 1508. None, one or both cage arrangements (diameter variable arrangement DVA) may be covered by a respective membrane. The membrane of the outer cannula 1508 may have an opening that faces laterally. The membrane of the outer cannula 1508 may have an opening that faces distally. In this case, cannula system 1500 may be used as cannula 1040.

FIGS. 7 to 12 show embodiments of cage arrangements that may be used in all of the embodiments shown in FIGS. 1A to 2D. FIG. 7 illustrates a cage arrangement 1600 comprising a membrane 1650 having an opening 1652 that faces distally. The shape of cage arrangement 1600 in its expanded state may be similar to a sphere or to a ball. Alternatively, an ellipsoid or another shape may be used. Cage arrangement 1600 is mounted to a cannula 1602 that may be an outer cannula or an inner cannula of a cannula system, for instance of one of cannula systems CS1 to CS3.

Cannula 1602 may comprise an optional cannula tip 1604 having apertures 1606, 1608 arranged in the pattern that is shown. However, other arrangements of apertures 1606, 1606 may be used, especially comprising a different number of apertures 1606, 1606. If cannula tip 1604 is not used there may be a single end-hole at the distal tip of cannula 1602. Cannula 1602 may not extend or may only extend by less than 10 mm into cage arrangement 1600.

If cage arrangement 1600 is viewed from above, it comprises in a counter clock wise direction eight cage wires 1610, 1612, etc. to 1624. More or less cage wires 1610 to 1624 may also be used. Cage wire 1624 is at the rear side of cage arrangement 1600. Cage wires 1610 to 1624 have, at a given axial position, same distances, especially same angularly distances, to the neighboring cage wires 1610 to 1624. One of the cage wires 1610 to 1624 or some of the cage wires 1610 to 1624 may be omitted, for instance to allow the insertion of further cannulas and/or cage arrangements through cage arrangement 1600.

Cage arrangement 1600 may have the following portions with increasing distance from mounting portion 1630:

a mounting portion 1630 at which the cage wires 1610 to 1624 are wound around distal end of cannula 1602, for instance at least three quarters of the circumference. Mounting portion 1630 may alternatively comprise an additional mounting element on which cage wires 1610 to 1624 are mounted, for instance a mounting sleeve or a jacket. In both cases a circumferential notch in cannula 1602 may be used to prevent axial movement of cage arrangement 1600 relative to cannula 1602. Additional or alternative mounting techniques may be used, i.e. welding, soldering, glue etc.

a proximal portion 1631 in which neighboring cage wires have increasing distances with regard to each other and with increasing distance to mounting portion 1630,

a comparably short optional transition portion 1632 in which cage wires 1610 to 1624 are arranged essentially parallel relative to each other and/or the longitudinal axis of cannula 1602 as well as to the longitudinal axis of cage arrangement 1600,

a distal portion 1633 in which neighboring cage wires have decreasing distances with regard to each other and with increasing distance to mounting portion 1630, and

a cage tip portion 1635 in which the cage wires 1610 to 1624 are connected together, for instance by a plastic cap. Within tip portion 1635 cage wires 1610 to 1624 may be twisted or be arranged parallel with regard to each other.

All cage wires 1610 to 1624 may be pre-bended in the same way and/or may have the same shape memorized within the shape memory of the material of the cage wires 1610 to 1624. An example is given for cage wire 1612 that is arranged mainly within a plane that is equal to the plane of the sheet that shows FIG. 7. Cage wire 1612 comprises portions that correspond to portions 1630 to 1635 of cage arrangement 1600:

a mounting portion 1640 that comprises:
an optional circumferential portion 1638 in which the wire is shaped circular, for instance along at least three quarters of the circumference of a circle, and
an optional straight portion 1639 that may be arranged parallel to the longitudinal axis of cage arrangement 1600 and that may serve as a reference axis in the following — alternatively the longitudinal axis of cannula 1602 may be uses as a reference axis,

a proximal portion 1641 in which wire 1612 has an increasing radial distance to the reference axis with increasing distance to mounting portion 1640,

an optional transition portion 1642 in which wire 1612 has a constant radial distance to the reference axis with increasing distance to mounting portion 1640,

a distal portion 1643 in which wire 1612 has a decreasing radial distance to the reference axis with increasing distance to mounting portion 1640, and

a cage tip portion 1645 that may be covered by plastic cap and/or in which the wire 1612 is parallel to the reference axis or is spirally and/or helically wounded.

Membrane 1650 extends circumferential from proximal P end almost up to distal D end of cage arrangement 1600, i.e. portions 1631 and 1632 are covered completely and portion 1633 is covered at more than half of its axial length. An opening 1652 faces distally to distal cage tip 1654 relative to longitudinal axis of cannula 1602 or of cage arrangement 1600.

Membrane 1650 may cover only or at least the lower half or only or at least the upper half of cage arrangement 1600, see line. In the latter case the opening of membrane 1650 would be facing proximally Membrane 1650 may cover also only the lower quarter or the lower three quarters of cage arrangement 1600. Reference may be made thereby to the axial length of cage arrangement 1600. Further, membrane 1650 may cover also only the upper quarter or the upper three quarters of cage arrangement 1600. However, cage arrangement 1600 may also be used without membrane 1600.

A separate spirally wounded wire that forms a coil may be used to form a mounting portion that is similar to mounting portion 1630. The number of windings within the coil may be in the range of 3 windings to 15 windings and/or in the range of 3 windings to 10 windings. There may be no space between adjacent or neighboring windings. Alternatively, there may be a small space between adjacent windings. The wires may have or may not have the circumferential portions, for instance 1638. The straight portions, for instance 1639, may be connected to the coil with the same axial position or with the same axial offset between angularly neighboring/adjacent wires, i.e. comparably to the arrangement that is shown in FIG. 7.

A sleeve may be used to form a mounting portion. The wires may have or may not have the circumferential portions, for instance 1638. The straight portions, for instance 1639, may be connected to the sleeve with the same axial position or with the same axial offset between angularly neighboring/adjacent wires, i.e. comparably to the arrangement that is shown in FIG. 7. The sleeve may be an inner sleeve relative to the wires or an outer sleeve. Furthermore, it is possible to use an inner sleeve and an outer sleeve with portions of the wires arranged therein between, especially straight portions that may be arranged in parallel or oblique to a longitudinal axis of the sleeve and/or of the cannula.

Other possibilities of the connection of the cage arrangement to the cannula may be used as well.

FIG. 8 illustrates a cage arrangement 1700 comprising a membrane 1750 having an opening that faces laterally. However, cage arrangement 1700 corresponds to cage arrangement 1600 except of the placement of membrane 1750. In order to avoid repetition reference is made to the description of FIG. 7 above. There are the following corresponding parts:

cage arrangement 1600, 1700,

cannula 1602, 1702,

optional cannula tip 1702, 1704,

apertures 1606, 1608, 1706, 1708,

cage wires 1610 to 1624, 1710 to 1724,

cage portions 1630 to 1635 are also valid for cage arrangement 1700,

circumferential portion 1638, 1738,

straight portions 1639, 1739,

cage wire portions 1640 to 1645 are also valid for cage arrangement 1700, and

cage tip 1654, 1754.

Features that are mentioned above for parts 1600 to 1645 apply also to the corresponding parts 1700 to 1739 and to the corresponding parts that are not indicated by reference signs in FIG. 17.

Membrane 1750 covers slightly more than half of cage arrangement 1700 and has an opening 1752 that faces laterally or transversally relative to the longitudinal axis of cannula I702 or of cage arrangement 1700. Thus, in the expanded state of cage arrangement 1700, membrane 1750 is arranged between cage wires 1718, 1720; 1720, 1722; 1722, 1724; 1724, 1710 and 1710, 1712. Membrane 1750 may extend through all main portions of cage arrangement 1700 between these cage wires, i.e. proximal portion, optional transition portion and distal portion, see corresponding portions 1631 to 1633 in FIG. 16.

Other arrangements of membrane 1750 are possible as well each having an opening that faces laterally:

less than 90 degrees in circumferential direction, preferably more than 10 degrees or more than 45 degrees,

90 degrees or more than 90 degrees of coverage in circumferential direction, but preferably less than 110 degrees, less than 135 degrees or less than 180 degrees,

180 degrees of coverage, i.e. membrane 1750 is only arranged between cage wires 1720 to 1724 and further between cage wire 1724 and 1710 as well as between cage wire 1710 and 1712, alternatively there may be at least 180 degrees of coverage, or

270 degrees of coverage, i.e. membrane 1750 is arranged additionally between cage wires 1716 and 1718, alternatively there may be at least 270 degrees of coverage.

Other angles of coverage for membrane 1750 may be easily realized if more or less than eight cage wires 1710 to 1724 are used in cage arrangement 1700. However, cage arrangement 1700 may also be used without a membrane. Combinations of lateral and distal/proximal facing openings are also possible.

A separate spirally wounded wire that forms a coil may be used to form a mounting portion that is similar to mounting portion 1730. The number of windings within the coil may be in the range of 3 windings to 15 windings and/or in the range of 3 windings to 10 windings. There may be no space between adjacent or neighboring windings. Alternatively, there may be a small space between adjacent windings. The wires may have or may not have the circumferential portions, for instance 1738. The straight portions, for instance 1739, may be connected to the coil with the same axial position or with the same axial offset between angularly neighboring/adjacent wires, i.e. comparably to the arrangement that is shown in FIG. 8.

A sleeve may be used to form a mounting portion. The wires may have or may not have the circumferential portions, for instance 1738. The straight portions, for instance 1739, may be connected to the sleeve with the same axial position or with the same axial offset between angularly neighboring/adjacent wires, i.e. comparably to the arrangement that is shown in FIG. 8. The sleeve may be an inner sleeve relative to the wires or an outer sleeve. Furthermore, it is possible to use an inner sleeve and an outer sleeve with portions of the wires arranged therein between, especially straight portions that may be arranged in parallel or oblique to a longitudinal axis of the sleeve and/or of the cannula.

Other possibilities of the connection of the cage arrangement to the cannula may be used as well.

FIG. 9 illustrates a cage arrangement 1800 comprising a portion that is bended backwards, i.e. backwards bended portion 1834. The shape of cage arrangement 1800 in its expanded state may be similar to a sphere or to a ball. Alternatively, an ellipsoid or another shape may be used. Cage arrangement 1800 is mounted to a cannula 1802 that may be an outer cannula or an inner cannula of a cannula system, for instance of one of cannula systems CS1 to CS3.

Cannula 1802 may comprise an optional cannula tip 1804 having apertures 1806, 1808 arranged in the pattern that is shown. However, other arrangement of apertures 1806, 1806 may be used, especially comprising a different number of apertures 1806, 1806. If cannula tip 1804 is not used, there may be a single end-hole at the distal tip of cannula 1802. Cannula 1802 may not extend or may only extend by less than 10 mm into cage arrangement 1800.

If cage arrangement 1800 is viewed from above, it comprises in a counter clock wise direction eight cage wires 1810, 1812, etc. to 1824. More or less cage wires 1810 to 1824 may also be used. Cage wire 1810 to 1824 is at the rear side of cage arrangement 1600. Cage wires 1810 to 1824 have at a given axial position same distances to the neighboring cage wires 1810 to 1824. One of the cage wires 1810 to 1824 or some of the cage wires 1810 to 1824 may be omitted, for instance to allow the insertion of further cannulas and/or cage arrangements through cage arrangement 1800.

Cage arrangement 1800 may have the following portions with increasing distance from mounting portion 1830:

a mounting portion 1830 at which the cage wires 1810 to 1824 are wound around distal end of cannula 1802, for instance at least three quarters of the circumference. Mounting portion 1830 may alternatively comprise an additional mounting element on which cage wires 1810 to 1824 are mounted, for instance a mounting sleeve or a jacket. In both cases a circumferential notch in cannula 1802 may be used to prevent axial movement of cage arrangement 1800 relative to cannula 1802. Additional or alternative mounting techniques may be used, i.e. welding, soldering, glue etc.

a proximal portion 1831 in which neighboring cage wires have increasing distances with regard to each other and with increasing distance to mounting portion 1830,

a comparably short optional transition portion 1832 in which cage wires 1810 to 1824 are arranged essentially parallel relative to each other and/or the longitudinal axis of cannula 1802 as well as to the longitudinal axis of cage arrangement 1800, and

a distal portion 1833 in which neighboring cage wires have decreasing distances with regard to each other and with increasing distance to mounting portion 1830.

There may be a short optional radial portion that forms a plane for contact with a wall of a vessel or a chamber of the heart. The radial length of this radial portion may be in the range of 3 mm to 10 mm (millimeters). In the expanded state, wire portions within the radial portion extend only radially but not axially, i.e. the wire portions have the same axial position and extend to the extended longitudinal axis of cannula 1802.

Furthermore, cage arrangement 1800 may comprise following the distal portion 1833:

a backwards bended portion 1834 in which cage wires 1810 to 1824 change direction and in which neighboring cage wires 1810 to 1824 have decreasing distances with regard to each other and with decreasing distance to mounting portion 1830, and

a cage tip portion 1835 in which the cage wires 1810 to 1824 are connected together, for instance by a plastic cap. Within tip portion 1835 cage wires 1810 to 1824 may be twisted or be arranged parallel with regard to each other.

All cage wires 1810 to 1824 may be pre bended in the same way and/or may have the same shape memorized within the shape memory of the material of the cage wires 1810 to 1824. An example is given for cage wire 1812 that is arranged mainly within a plane that is equal to the plane of the sheet that shows FIG. 9. Cage wire 1812 comprises portions that correspond to portions 1830 to 1835 of cage arrangement 1800:

a mounting portion 1840 that comprises:
an optional circumferential portion 1838 in which the wire is shaped circular, for instance along at least three quarters of the circumference of a circle, and
an optional straight portion 1839 that may be arranged parallel to the longitudinal axis of cage arrangement 1800 and that may serve as a reference axis in the following—alternatively the longitudinal axis of cannula 1802 may be uses as a reference axis,

a proximal portion 1841 in which wire 1812 has an increasing radial distance to the reference axis with increasing distance to mounting portion 1840,

an optional transition portion 1842 in which wire 1812 has a constant radial distance to the reference axis with increasing distance to mounting portion 1840, and

a distal portion 1843 in which wire 1812 has a decreasing radial distance to the reference axis with increasing distance to mounting portion 1840.

There may be the optional radial portion that is mentioned above. The optional radial portion may be arranged between the distal portion 1843 and the backwardly bended wire portion 1844.

Furthermore cage wire 1812 may comprise following the distal portion 1843:

a backwardly bended wire portion 1844, and

a cage tip portion 1845 that may be covered by plastic cap and/or in which the wire 1812 is parallel to the reference axis or is spirally and/or helically wounded.

In another embodiment cage arrangement 1800 may be covered at least partially by a membrane. The membrane may have an opening that faces distally or proximally, see description of FIG. 7, for instance coverage of lower three quarters. Alternatively, the membrane may have an opening that faces laterally, see description of FIG. 8, for instance coverage of at least half of the circumference or of three quarter of circumference. Combinations of lateral and distal/proximal facing openings are also possible.

Other shapes of cage arrangements with a backward bended portion are also possible, see for instance shapes similar to the shapes that are shown in FIGS. 10 and 11, e.g. cone shape or cylinder shape.

A separate spirally wounded wire that forms a coil may be used to form a mounting portion that is similar to mounting portion 1830. The number of windings within the coil may be in the range of 3 windings to 15 windings and/or in the range of 3 windings to 10 windings. There may be no space between adjacent or neighboring windings. Alternatively, there may be a small space between adjacent windings. The wires may have or may not have the circumferential portions, for instance 1838. The straight portions, for instance 1839, may be connected to the coil with the same axial position or with the same axial offset between angularly neighboring/adjacent wires, i.e. comparably to the arrangement that is shown in FIG. 9.

A sleeve may be used to form a mounting portion. The wires may have or may not have the circumferential portions, for instance 1838. The straight portions, for instance 1839, may be connected to the sleeve with the same axial position or with the same axial offset between angularly neighboring/adjacent wires, i.e. comparably to the arrangement that is shown in FIG. 9. The sleeve may be an inner sleeve relative to the wires or an outer sleeve. Furthermore, it is possible to use an inner sleeve and an outer sleeve with portions of the wires arranged therein between, especially straight portions that are arranged in parallel or oblique to a longitudinal axis of the sleeve and/or of the cannula.

Other possibilities of the connection of the cage arrangement to the cannula may be used as well.

FIG. 10 illustrates cannulas 1908, 1910 of a cannula system 1900. Cannula 1908 is an outer cannula 1908 of cannula system 1900. Cannula 1910 is an inner cannula 1910 of cannula system 1900. The length and/or outer diameters of cannulas 1908 and 1910 may be different, i.e. inner cannula 1910 may be longer and thinner than outer cannula 1908. Disregarding these differences, both cannulas 1908 and 1910 may correspond to the picture that is shown in FIG. 10. One of the cannulas 1908, 1910 or both cannulas 1908, 1910 may comprise a cage arrangement 1912.

Cannula system 1900 may be adapted for a stepwise insertion of the cannulas 1908 and 1910, i.e. first outer cannula 1908 and then inner cannula 1910. Dual lumen cannula system 1900 may comprise, preferably as separate systems for each cannula 1908 and 1910 or only one system for both cannulas 1908 and 1910:

a locking mechanism 1902 that may lock an introducer that is used for introducing outer cannula 1908 or inner cannula 1910, especially after outer cannula 1908 has been introduced. FIG. 12 shows an example in which an introducer or introducer member is inserted into a cannula system. The introducer may be locked by force fitting or by another appropriate mechanism.

an adapter portion 1904 that may be used to connect locking mechanism 1902 removably to cannula system 1900, and

a handle portion 1906 that may also be connected removably to cannula system 1900 and that is used to ease introducing of outer cannula 1908 or inner cannula 1910 into body 100 of a patient.

Inner cannula 1908 and/or outer cannula 1910 may comprise a cage arrangement 1912 having wires that are essentially arranged in parallel with regard to each other in the main portion of the cage arrangement 1912, e.g. along the entire axial length of cage arrangement 1912 or along at least 90 percent of this length. Thus cage arrangement 1912 has the shape of a cylinder.

Cage arrangement 1912 of inner cannula 1908 and/or outer cannula 1910 may have or may comprise a membrane, for instance a membrane that has an opening facing distally or laterally, see for instance FIG. 7 and FIG. 8 and corresponding descriptions.

Cannula 1908 and/or cannula 1910 may be pre bended as described above for cannula system 1900. The tip of cannula 1908 and/or 1909 may be optional, i.e. there may be only one opening or end-hole within cage arrangement 1912, for instance at its proximal end.

The cage arrangement 1912 may be used also for a single lumen cannula, preferably with or without a membrane.

FIG. 11 illustrates cannulas 2008, 2010 of a cannula system 2000 comprising a cage arrangement 2012 having a cone like shape. Cannula 1908 is an outer cannula 1908 of cannula system 1900. Cannula 2010 is an inner cannula 2010 of cannula system 2000. The length and/or outer diameters of cannulas 1908 and 1910 may be different, i.e. inner cannula 1910 may be longer and thinner than outer cannula 1908. Disregarding these differences, both cannulas 1908 and 1910 may correspond to the picture that is shown in FIG. 11. One of the cannulas 2008, 2010 or both cannulas 2008, 2010 may comprise a cage arrangement 2012.

Cannula system 2000 may be adapted or is adapted for a stepwise insertion of cannulas 2008 and 2010, i.e. first outer cannula 2008 and then inner cannula 2010. Dual lumen cannula system 2000 may comprise, preferably as separate systems for each cannula 2008 and 2010 or only one system for both cannulas 2008 and 2010:

a locking mechanism 2002 that may lock an introducer or introducer member that is used for introducing outer cannula 2008 or inner cannula 2010, especially after outer cannula 2008 has been introduced. FIG. 12 shows an example in which an introducer is inserted into a cannula system 2000. The introducer may be locked by force fitting or by another appropriate mechanism.

an adapter portion 2004 that may be used to connect locking mechanism 2002 removably to cannula system 2000, and

a handle portion 2006 that may also be connected removably to cannula system 2000 and that is used to ease introducing of outer cannula 2008 or inner cannula 2010 into body 100 of a patient.

Inner cannula 2008 and/or outer cannula 2010 may comprise a cage arrangement 2012 having wires that extend in the proximal portion of cage arrangement 2012 essentially radially outward. Within an optional short transition portion of cage arrangement 2012 the wires are parallel to each other and/or to the longitudinal axis of cannula 2008, 2010. Within a distal portion of cage arrangement 2012 the wires are arranged on a surface that would define the inclined surface of a cone. This distal portion may extend along almost the entire axial length of cage arrangement 2012 or along at least 90 percent of this length. Thus, it may be said that cage arrangement 2012 has the shape of a cone. Within the distal portion of cage arrangement 2012 the distances between neighboring wires are decreasing with increasing distance to a mounting portion of cage arrangement 2012.

Cage arrangement 2012 of inner cannula 2008 and/or outer cannula 2010 may have or may comprise a membrane, for instance a membrane that has an opening facing distally or laterally, see for instance FIG. 7 and FIG. 8 and corresponding descriptions.

Cannula 2008 and/or cannula 2010 may be pre-bended as described above for cannula system 2000. The tip of cannula 2008 and/or 2009 may be optional, i.e. there may be only one opening within cage arrangement 2012, for instance at its proximal end.

The cage arrangement 2012 may be used also for a single lumen cannula, preferably with or without a membrane.

FIG. 12 illustrates cannula system 2000 in a state in which an introducer 2114 (introducer member) stretches the cage arrangement 2012 for introducing cannula 2008 or 2010 into body 100. Introducer 2114 may also be named as mandrel. Introducer 2114 has a proximal end 2114p and a distal end 2114d. Proximal end 2114p may be clamped within locking mechanism 2002 in the position in which cage arrangement 2012 is stretched enabling the practitioner or the physician to fully concentrate on careful introduction of cannula system 2000 into body 100. Distal end 2114d may be adapted to engage with cage arrangement 2012, preferable with the distal tip and/or the distal portion of cage arrangement 2012. Distal end 2114d may be tapered. If cannula 2008, 2010 is in place, introducer 2114 is unlocked by operating locking mechanism 2002. Thereafter, introducer 2114 is pulled out of cannula 2008, 2010.

At the end of the medical treatment, introducer 2114 may be used again to stretch cage arrangement 2012 and to remove cannula 2008, 2010 out of body 100.

The diameter of introducer 2114 is adapted to have only small slit/gap between an outer surface of introducer 2114 and an inner surface of cannula 2008, 2010. The slit/gap may be smaller than 0.5 millimeter or smaller than 250 micron (micrometer). However, the slit/gap may be greater than 100 micron to allow axial movement of introducer 2114 within cannula 2008, 2010.

Alternatively, introducer 2114 may also be used for cannula system 1900 or for other cannula systems, for instance the cannula systems that are shown in FIGS. 1A to 2E. Introducer 2144 may also be used for cage arrangements (diameter variable arrangements) that comprise a membrane, see for instance FIGS. 7 and 8. An adapted introducer may be used to introduce cannulas comprising cage arrangement 1800 that is shown in FIG. 9.

FIG. 13 illustrates an alternative embodiment of a circuitry 2206 wherein a cannula 2240a is pierced or punctured through a ventricle septum VS of heart H. Circuitry 2206 is extracorporeal. Circuitry 2206 may comprise or may consist of:

single lumen cannula 2240a, inserted preferably endovascular jugular,

a single lumen cannula 2240b, inserted preferably endovascular jugular,

a pump P22, and

an oxygenator OXY22.

A proximal end of cannula 2240b may be connected to an input port of oxygenator OXY22 via a flexible tube 2220. An output port of oxygenator OXY22 may be connected to an input port of pump P22, for instance via a tube 2240. An outlet port of pump P22 may be connected to a proximal end of cannula 2240a.

Cannula 2240a may carry a cage arrangement 2246 at its distal end. Cage arrangement 2246 may be placed within ascending aorta aAO. Cage arrangement 2246 may comprise a membrane, for instance a membrane having an opening that faces distally. Alternatively, cage arrangement 2246 may not have a membrane. Cannula 2240a may be inserted endovascular through left internal jugular vein IJV, superior vena cava SVC, right atrium RA, right ventricle RV, ventricle septum VS, left ventricle LV up to ascending aorta aAO. Especially cage arrangement 2246 may be placed within ascending aorta aAO. Only a small part of the distal end of cannula 2240a may be located within cage arrangement 2246 and therefore also within ascending aorta aAO, for instance less than 5 mm Thus, cage arrangement 2246 does not comprise a separate distal tip (for instance made of a different material compared to the material of cannula 2240a) that has lateral side holes and/or a distal end-hole. However, in an alternative embodiment, cage arrangement 2246 may comprise a distal tip that has lateral side holes and/or a distal end-hole.

Cannula 2240b may carry a cage arrangement 2286 at its distal end. Cage arrangement 2286 may be placed within left atrium LA preferably transseptal through the atrial septum AS of the heart H. Cage arrangement 2286 may comprise a membrane, for instance a membrane having an opening that faces laterally. Alternatively, cage arrangement 2286 may not have a membrane. Cannula 2240b may be inserted endovascular through right internal jugular vein IJV, superior vena cava SVC and atrial septum AS into left atrium LA. Especially cage arrangement 2286 may be placed within left atrium LA. Only a small part of the distal end of cannula 2240b may be located within cage arrangement 2286 and therefore also within left atrium LA, for instance less than 5 mm Thus, cage arrangement 2286 does not comprise a separate distal tip (for instance made of a different material compared to the material of cannula 2240b) that has lateral side holes and/or a distal end-hole. However, in an alternative embodiment, cage arrangement 2286 may comprise a distal tip that has lateral side holes and/or a distal end-hole.

Alternatively, it is possible to insert cannula 2240b through right internal jugular vein rIJV and cannula 2240b through left internal jugular vein WV. Guide wires and/or introducer members may be used in all cases for the insertion of cannulas 2240a and 2240b.

Pump P22 may drive a drainage flow that comes in through all four pulmonary (see arrows 2297) veins PV out of left atrium LA, through cannula 2240b, tube 2220, oxygenator OXY22, tube 2240, pump P22, tube 2230 and finally through cannula 2240a into ascending aorta aAO, see arrow 2270. Pump P22 may be operated in pulsed mode or may be a pump that generates a pulsatile blood flow, for instance a roller pump. Synchronization to the diastole and systole phases of heart H pumping is possible if a sensor is used, for instance a blood pressure sensor. Alternatively, blood pump P22 may generate a continuous blood flow.

Alternatively, cannula 2240a may be a dual lumen cannula or a multi lumen cannula. Cannula 2240b may be omitted if cannula 2240a is a dual lumen cannula or a single lumen cannula. If cannula 2240b is omitted and if cannula 2240a is a dual lumen cannula the outer cannula may be used to drain blood from left ventricle LV or to deliver blood, see FIG. 14 and corresponding description.

The ventricle septum VS may be a preferred place for puncturing, for instance if the atrial septum AS may not be used. Other medical devices may be placed within atrial septum AS or it may have been punctured too often. There may also be a disease of the atrial septum AS. However, even without special reasons the ventricle septum VS may be used and not the atrial septum AS.

The ventricle septum VS may be used for instance in variants of the following embodiments:

in the embodiment that is shown in FIG. 1A for cannula 910,

in the embodiment that is shown in FIG. 1B for cannula L2b,

in the embodiment that is shown in FIG. 1C for cannula L2c,

in the embodiment that is shown in FIG. 2A for cannula 1040,

in the embodiment that is shown in FIG. 2B for cannula 1040,

in the embodiment that is shown in FIG. 2C for cannula 1040,

in the embodiment that is shown in FIG. 2D for cannula L2d, and

in the embodiment that is shown in FIG. 2E for cannula L2e.

It may be possible to avoid two cannulas within the right ventricle RV if the main pulmonary artery PA or especially the right pulmonary artery rPA or the left pulmonary artery IPA are reached transcaval from vena cava VC, especially from superior vena cava SVC by puncturing of the vena cava VC and of the respective pulmonary artery PA, IPA, rPA, see for instance FIG. 16. The cannula that takes the transcaval “short cut” may be inserted endovascular jugular, preferably through one of the internal jugular veins UV, e.g. left internal jugular vein lIJV or right internal jugular vein rIJV. However, endovascular femoral access to vena cava VC and then transcaval to one of the pulmonary arteries PA, IPA, rPA is possible as well. The transcaval cannula may be a single lumen cannula.

FIG. 14 illustrates a further alternative embodiment of a circuitry 2306 wherein a dual lumen cannula 2310 is pierced or punctured through ventricle septum VS. Circuitry 2306 is extracorporeal. Circuitry 2306 may comprise or may consist of:

multi lumen or dual lumen cannula 2310, inserted preferably endovascular jugular,

a pump P23, and

an oxygenator OXY23.

A proximal end of the outer cannula of dual lumen cannula 2310 may be connected to an input port of pump P23 via a flexible tube 2320. An output port of pump P23 may be connected to an input port of oxygenator OXY23, for instance via a tube 2340. An outlet port of oxygenator OXY23 may be connected to a proximal end of the inner cannula of dual lumen cannula 2310.

The inner cannula of dual lumen cannula 2310 may carry a cage arrangement 2346 at its distal end. Cage arrangement 2346 may be placed within ascending aorta aAO. Cage arrangement 2346 may comprise a membrane, for instance a membrane having an opening that faces distally. Alternatively, cage arrangement 2346 may not have a membrane.

Cannula 2310 may be a fixed dual lumen cannula or a non-fixed dual lumen cannula. A fixed dual lumen cannula 2310 may be inserted endovascular through right internal jugular vein IJV or left internal jugular vein IJV, superior vena cava SVC, right atrium RA, right ventricle RV, ventricle septum VS, left ventricle LV up to ascending aorta aAO. Especially cage arrangement 2346 may be placed within ascending aorta aAO. Only a smart part of the distal end of the inner cannula of cannula 2310 may be located within cage arrangement 2346 and therefore within ascending aorta aAO, for instance less than 5 mm Thus, cage arrangement 2346 does not comprise a separate distal tip (for instance made of a different material compared to the material of the inner cannula of dual lumen cannula 2310 that has lateral side holes and/or a distal end-hole. However, in an alternative embodiment, cage arrangement 2346 may comprise a distal tip that has lateral side holes and/or a distal end-hole.

The outer cannula of dual lumen cannula 2310 may have an inlet portion 2390 comprising a group of inlet holes, for instance a number of holes within the range from 4 to 20. Inlet portion 2390 may be placed within the right atrium RA if cannula 2310 is in its final position within heart H. Additionally or alternatively, the outer cannula of dual lumen cannula 2310 may have an inlet portion 2398 comprising a group of inlet holes, for instance a number of holes within the range from 4 to 20. Inlet portion 2398 may be placed within the right ventricle RV if cannula 2310 is in its final position within heart H. A group of holes may also be arranged within superior vena cava SVC in addition to at least one of the group of holes mentioned above.

Alternatively, a group of holes may be arranged within the left ventricle for delivery or drainage of blood.

There may be an optional cage arrangement around inlet portion 2390. An outer sheet member may be used to hold this cage arrangement in its closed or non-expanded state during insertion of dual lumen cannula. An optional cage arrangement may be used around inlet portion 2398. An outer sheet member may be used to hold this cage arrangement in its closed or non-expanded state during insertion of dual lumen cannula if a fixed dual lumen cannula is used. For a non-fixed dual lumen cannula it is possible to use an introducer member to bring the cage arrangement around inlet holes 2398 in its non-expanded state.

Alternatively, if cannula 2310 is a non-fixed dual lumen cannula it is possible to insert outer cannula first, i.e. through left internal jugular vein lIJV or right internal jugular vein rIJV, through vena cava VC, right atrium RA up to left ventricle LV. After the insertion of outer cannula of dual lumen cannula 2310 inner cannula is inserted through outer cannula and then through ventricle septum VS, left ventricle LV and up to ascending aorta aAO.

Guide wires and/or introducer members may be used in all cases for the insertion of cannula 2310 in one step or in two single steps that are performed in sequence, i.e. first insertion of outer cannula and then insertion of inner cannula.

Pump P23 drives a drainage flow from right atrium (see arrow 2392) and/or from right ventricle RV (see arrow 2399) through outer cannula of dual lumen cannula 2310, tube 2320, pump P23, tube 2340, oxygenator OXY23, tube 2330 and finally through inner cannula of dual lumen cannula 2310 into ascending aorta aAO, see arrow 2370. Pump P23 may be operated in pulsed mode or may be a pump that generates a pulsatile blood flow, for instance a roller pump. Synchronization to the diastole and systole phases of heart pumping is possible if a sensor is used, for instance a blood pressure sensor. Alternatively, blood pump P23 may generate a continuous blood flow.

The ventricle septum VS may be a preferred place for puncturing, for instance if the atrial septum AS may not be used. Other medical devices may be placed within atrial septum AS or it may have been punctured too often. However, even without a special reason the ventricle septum VS may be used.

The ventricle septum VS may be used for instance in variants of the following embodiments:

in the embodiment that is shown in FIG. 1A for the insertion of cannula 910,

in the embodiment that is shown in FIG. 1B for the insertion of cannula L2b,

in the embodiment that is shown in FIG. 1C for the insertion of cannula L2c, cannula L3c may be realized by outer cannula of cannula 2310, i.e. no dual lumen cannula DLc may be used

in the embodiment that is shown in FIG. 2A for cannula 1040, the inlet portion of outer cannula of cannula 2310 may be arranged within left atrium LA and used as an outlet portion,

in the embodiment that is shown in FIG. 2B for cannula 1040, the inlet portion of outer cannula of cannula 2310 may be arranged within left atrium LA,

in the embodiment that is shown in FIG. 2C for cannula 1040, the inlet portion of outer cannula of cannula 2310 may be arranged within left atrium LA,

in the embodiment that is shown in FIG. 2D for cannula L2d, cannula L3d may be realized by outer cannula of cannula 2310, i.e. no dual lumen cannula DLd may be used, and

in the embodiment that is shown in FIG. 2E for cannula L2e, cannula L3e may be realized by outer cannula of cannula 2310, i.e. no dual lumen cannula DLe may be used.

It is possible to avoid two cannulas within the right ventricle RV if the main pulmonary artery PA or especially the right pulmonary artery rPA or the left pulmonary artery IPA are reached transcaval from vena cava VC, especially from superior vena cava SVC by puncturing of the vena cava VC and of the respective pulmonary artery PA, IPA, rPA. The cannula that takes the transcaval “short cut” may be inserted endovascular jugular, preferably through one of the internal jugular veins IJV, e.g. left internal jugular vein lIJV or right internal jugular vein rIJV. However, endovascular femoral access to vena cava VC and then transcaval to one of the pulmonary arteries PA, IPA, rPA is possible as well. The transcaval cannula may be a single lumen cannula.

FIG. 15 illustrates an alternative embodiment of a circuitry 2406 wherein a cannula L15b may be punctured transcaval from vena cava VC to the aorta AO. Circuitry 2406 may comprise or consist of:

a single lumen cannula L15a,

single lumen cannula L15b,

a pump P15, and

an optional oxygenator OXY.

A proximal end of cannula L15a may be connected to an inlet port of pump P15, for instance via a flexible tube. An outlet port of pump P15 may be connected to the proximal end of cannula L15b, for instance via a flexible tube. An oxygenator OXY and/or a carbon dioxide removal unit and/or an adsorber/filter unit and/or another medical device may be included within circuitry 2406 at an appropriate location.

Cannula L15a may be a single lumen cannula that carries at least one expandable arrangement, for instance a cage arrangement, especially a cage arrangement as describes above. If a cage arrangement is used, a membrane may be used as well that is connected to the cage arrangement. However, at least one cage arrangement without a membrane may be used alternatively.

Cannula L15a may be inserted endovascular through left internal jugular vein lIJV or through right internal jugular vein rIJV or through another appropriate vessel. Cannula L15a is farther inserted through vena cava VC into the right atrium RA and/or into the right ventricle RV. An inlet portion comprising a group of inlet holes may be arranged within right atrium RA on cannula L15a. Alternatively or additionally, an inlet portion comprising a group of inlet holes may be arranged within right ventricle RV on cannula I5a.

Cannula L15b may be a single lumen cannula that carries an expandable arrangement, for instance a cage arrangement, especially a cage arrangement as describes above, or a balloon, see description of FIGS. 17 and 18 below. If a cage arrangement is used, a membrane may be used as well that is connected to the cage arrangement. The membrane may have an opening that faces distally with regard to the longitudinal axis of cannula L15b.

Cannula L15b may be inserted endovascular through left internal jugular vein lIJV or through right internal jugular vein rIJV or through another appropriate vessel. Cannula L15b is farther inserted through vena cava VC, especially through superior vena cava SVC, through a hole within the wall of vena cava VC, especially a hole in superior vena cava SVC, transcaval to a hole within ascending aorta aAO up to the ascending aorta aAO, where it is fixed for instance by the expandable arrangement mentioned above.

Pump P15 may drive a drainage flow from right atrium RA (see arrow) and/or from right ventricle RV through cannula L15a, pump P15 and finally through cannula L15b into ascending aorta aAO, see arrow. Pump P15 may be operated in pulsed mode or may be a pump that generates a pulsatile blood flow, for instance a roller pump. Synchronization to the diastole and systole phases of heart pumping is possible if a sensor is used, for instance a blood pressure sensor. Alternatively, blood pump P15 may generate a continuous blood flow.

Optional oxygenator OXY may increase the oxygen content of the blood extracorporeal. Thereby, carbon dioxide may be removed.

Alternatively a split tip cannula may be used that comprises both cannulas L15a and L15b, especially a split tip dual lumen cannula wherein the inner cannula may comprise alt least two distal tips.

The atrial septum AS and/or the ventricle septum VS may not be a preferred place for puncturing, for instance if other medical devices are placed within the atrial septum AS and/or ventricle septum VS or if one of these septa has or both have been punctured too often. There may also be a disease affecting one or both of the atrial septum AS and/or of the ventricle septum VS. Furthermore, the proposed transcaval shortcut from vena cava VC, preferably from superior vena cava SVC, to ascending aorta aAO may be used if the valves of heart H do not function properly any more, for instance because of a disease. However, even without special reasons the shortcut to the aorta AO may be chosen and not a way through one of the septa AS or VS.

The following embodiments may be modified:

FIG. 2A, cannula L15b may be used for inner cannula of dual lumen cannula 1040, outer cannula of dual lumen cannula 1040 may still be used as a single lumen cannula,

FIG. 2B, cannula L15b may be used for inner cannula of dual lumen cannula 1040, outer cannula of dual lumen cannula 1040 may still be used as a single lumen cannula, and

FIG. 2C, cannula L15b may be used for inner cannula of dual lumen cannula 1040, outer cannula of dual lumen cannula 1040 may still be used as a single lumen cannula.

FIG. 16 illustrates a further alternative embodiment wherein a cannula L16b is punctured or pierced transcaval from vena cava VC to main pulmonary artery PA or to right pulmonary artery rPA or to left pulmonary artery IPA. A circuitry 2506 may comprise or consist of:

a single lumen cannula L16a,

single lumen cannula L16b,

a pump P16, and

an optional oxygenator OXY.

A proximal end of cannula L16a may be connected to an inlet port of pump P16, for instance via a flexible tube. An outlet port of pump P16 may be connected to the proximal end of cannula L16b, for instance via a flexible tube. An oxygenator OXY and/or a carbon dioxide removal unit and/or an adsorber/filter unit and/or another medical device may be included within circuitry 2506 at an appropriate location.

Cannula L16a may be a single lumen cannula that carries at least one expandable arrangement, for instance a cage arrangement, especially a cage arrangement as describes above. If a cage arrangement is used, a membrane may be used as well that is connected to the cage arrangement. However, at least one cage arrangement without a membrane may be used alternatively.

Cannula L16a may be inserted endovascularly through left internal jugular vein lIJV or through right internal jugular vein rIJV or through another appropriate vessel. Cannula L16a is farther inserted through vena cava VC into the right atrium RA and/or into the right ventricle RV. An inlet portion comprising a group of inlet holes may be arranged within right atrium RA on cannula L16a. Alternatively or additionally, an inlet portion comprising a group of inlet holes may be arranged within right ventricle RV on cannula L16a.

Cannula L16b may be a single lumen cannula that carries an expandable arrangement, for instance a cage arrangement, especially a cage arrangement as describes above, or a balloon, see description of FIGS. 17 and 18 below. If a cage arrangement is used, a membrane may be used as well that is connected to the cage arrangement. The membrane may have an opening that faces distally with regard to the longitudinal axis of cannula L16b.

Cannula L16b may be inserted endovascular through left internal jugular vein lIJV or through right internal jugular vein rIJV or through another appropriate vessel. Cannula L16b is farther inserted through vena cava VC, especially through superior vena cava SVC, through a hole within the wall of vena cava VC, especially a hole in superior vena cava SVC, transcaval to a hole within main pulmonary artery PA or to right pulmonary artery rPA or to left pulmonary artery IPA up to main pulmonary artery PA or to right pulmonary artery rPA or to left pulmonary artery lPA, where it is fixed for instance by the expandable arrangement mentioned above.

Pump P16 may drive a drainage flow from right atrium RA (see arrow) and/or from right ventricle RV through cannula L16a, pump P16 and finally through cannula L16b into main pulmonary artery PA, right pulmonary artery rPA or left pulmonary artery, see arrow. Pump P16 may be operated in pulsed mode or may be a pump that generates a pulsatile blood flow, for instance a roller pump. Synchronization to the diastole and systole phases of heart pumping is possible if a sensor is used, for instance a blood pressure sensor. Alternatively, blood pump P16 may generate a continuous blood flow.

Optional oxygenator OXY may increase the oxygen content of the blood extracorporeal. Thereby, carbon dioxide may be removed.

Alternatively a split tip cannula may be used that comprises both cannulas L16a and L16b, especially a split tip dual lumen cannula wherein the inner cannula comprises at least two tips.

The atrial septum AS and/or the ventricle septum VS may not be a preferred place for puncturing, for instance if other medical devices are placed within the atrial septum AS and/or ventricle septum VS or if one of these septa AS, VS has or both have been punctured too often. There may also be a disease affecting one or both of the atrial septum AS and/or of the ventricle septum VS. Furthermore, the proposed transcaval shortcut from vena cava VC, preferably from superior vena cava SVC, to main pulmonary artery PA, to right pulmonary artery rPA or to left pulmonary artery IPA may be used if the valves of heart H do not function properly any more, for instance because of a disease. However, even without special reasons shortcut to the pulmonary artery (PA, rPA, IPA) may be chosen and not a way through one of the septa AS, VS.

The following embodiments may be modified for instance:

in the embodiment that is shown in FIG 1A cannula L16b may be used for cannula 910, cannula L16a is not necessary,

in the embodiment that is shown in FIG. 1B cannula L16b may be used for cannula L2b, cannula L16a is not necessary,

in the embodiment that is shown in FIG. 1C cannula L16b may be used for cannula L2c, cannula L3c may be realized by cannula L16a, i.e. no dual lumen cannula DLc may be used,

in the embodiment that is shown in FIG. 2A cannula L16b may be used for inner cannula of dual cannula 1010, the inlet portion(s) 1090 and/or 1098 of outer cannula of cannula 1010 may still be used, cannula L16a is not necessary,

in the embodiment that is shown in FIG. 2B cannula L16b may be used for inner cannula of dual cannula 1010, the inlet portions 1090 and/or 1098 of outer cannula of cannula 1010 may still be used, cannula L16a is not necessary,

in the embodiment that is shown in FIG. 2C cannula L16b may be used for inner cannula of dual for cannula 1010, the inlet portion s 1090 and/or 1098 of outer cannula of cannula 1010 may still be used, cannula L16a is not necessary,

in the embodiment that is shown in FIG. 2D cannula L16b may be used for cannula L1d, cannula L3d may be realized by cannula L16a, i.e. no dual lumen cannula DLd may be used, and

in the embodiment that is shown in FIG. 2E cannula L16b may be used for cannula L1e, cannula L3e may be realized by cannula L16a, i.e. no dual lumen cannula DLe may be used.

FIG. 17 illustrates a cannula L17 that carries an inflatable expandable arrangement Ba, for instance a balloon. Balloon Ba may have a cylindrical shape and may be connected to a distal portion of cannula L17, for instance using an adhesive.

A channel CH1 may be arranged on an outer surface of cannula L17. Channel CH1 may extend from a proximal part of cannula L17 up to balloon Ba. If a fluid is driven into channel CH1 balloon Ba inflates. If the fluid is driven out of channel CH1 then balloon Ba deflates.

Thus, balloon Ba may form a border element that is between cannula L17 and a vessel V of the blood circuit. Vessel V may be a pulmonary artery PA, IPA, rPA of lung L or a pulmonary vein PV, IPV or rPV of lung L. There may be a transport volume TrV that is used to treat lung L and that is on the distal side of inflated balloon Ba. The natural blood circuit BC may be on the proximal side of balloon Ba. Balloon Ba may isolate the natural blood circuit BC from transport volume TrV.

Transport volume TrV may be directly in fluidic connection with cannula L17 through the holes in a separate distal tip Til7, see for instance holes Hol7. Alternatively, cannula L17 may have only a single end-hole EH at its distal end.

Cannula L17 may be one of the cannulas L1b, L1c, L1d, L1e, L2b, L2c, L2d, L2e or one of the other cannulas mentioned above during the description of FIG. 1A, 2A, 2B, 2C, 3 to 6, 13 or 14.

Alternatively and/or additionally, channel CH1 may be arranged within cannula L17. Cannula L17 may be a single lumen cannula or a multi lumen cannula, especially the inner cannula or the outer cannula of a dual lumen cannula. A combination of an internal channel and an external channel is possible as well.

Deflated balloon Ba may not have a further protection shield during insertion of cannula L17. However, alternatively a removable sheath may be wrapped around balloon Ba during insertion of cannula L17.

FIG. 18 illustrates a split tip cannula L18 that carries two expandable arrangements on its two distal end lumens L18a and L18b. An inner lumen of cannula L18 bifurcates in distal end lumen L18a and distal end lumen L18b at a bifurcation point or bifurcation region Bi. Each of the expandable arrangements may be a balloon Ba as shown in FIG. 17 and described above. A channel CH2 may correspond to channel CH1 mentioned above. Channel CH2 may be connected to a channel CH2a that extends on distal end lumen L18a to the respective balloon and to a channel CH2b that extends on distal end lumen L18b to the respective balloon. Alternatively or additionally, internal channels may be used to inflate or deflate the balloons. A combination of an internal channel and an external channel is possible as well. Furthermore, it is possible to use two separate channels CH2a1 and CH2b1 that extend from a proximal part of cannula L18 to either distal end lumen L18a or distal end lumen L18b. Separate and independent control of balloon on distal end lumen L18a and on distal end lumen L18b is possible in this variant. Introduction and fixation of cannula L18 may be easier with separate control of both balloons.

Alternatively two cage arrangements may be used on the distal end lumens L18a and L18b. Cage arrangements that are mentioned above may be used, see for instance FIGS. 1A, 2A, 6 to 12, 13 and 14. An introducer member 118 may be used that has a split tip, i.e. a bifurcation. Alternatively two separate introducer members may be used within cannula L18, one extending into distal end lumen L18a and the other extending into distal end lumen L18b.

Cannula L18 may be a single lumen cannula having a split tip. Alternatively, cannula L18 may comprise two separate lumens, one connected to lumen L18a and the other connected to lumen L18b.

Furthermore, for each embodiment of cannula L18 described above cannula L18 may not be inserted into a further cannula or may be inserted in a further cannula—thus forming a dual lumen cannula, for instance in a fixed dual lumen cannula or multi lumen cannula or non-fixed cannula dual lumen cannula or multi lumen cannula.

FIG. 19 illustrates an embodiment of a lung perfusion system 2600 in combination with an inhalation system INH for treating only one lobe Lo2 of lung L. Lung L is illustrated in a posterior view. Therefore, a left half lH of lung L is shown on the right side of FIG. 19 and a right half rH of lung L is shown on left side of FIG. 19. There are the following lobes of lung L in right half rH:

an upper lobe Lo1 (apical lobe),

a middle lobe Lo2, and

a lower lobe Lo3.

There are the following lobes of lung L in left half lH:

an upper lobe Lo4, and

a lower lobe Lo5, i.e. there is no middle lobe at left half lH of lung L.

The trachea Tr is the main air channel of lung L and extends from throat to a lower bifurcation. At the lower bifurcation trachea Tr branches in left primary bronchus Brla and right primary bronchus Brlb. A tube Tu is arranged in trachea Tr and extends through trachea Tr into right primary bronchus Brlb but not into left primary bronchus Brla or vice versa. Tube Tu is inserted for instance through the mouth of a patient, through an incision in trachea Tr or in another way.

Primary left bronchus Brla has at least one further bifurcation (not shown) where it branches in secondary bronchi which branch further in tertiary bronchi and then in small bronchi of the left half lH of lung L. Primary right bronchus Brlb has at least one further bifurcation (not shown) where it branches in secondary bronchi which branch further in tertiary bronchi and then in small bronchi of the right half rH of lung L.

An arrow Ar19 illustrates an example in which only the alveoli of middle lobe Lo2 are brought into contact with a medicament M which is inhaled by the patient through tube Tu. Instead of inhalation other transport methods may be used for instance dropping a liquid into tube Tu which liquid comprises the treatment substance M. A liquid flow in the blood vessel system of lung L may be used to remove the medicament from the blood or from another auxiliary liquid, for instance saline solution. This allows using very high doses of the medicament which is transported through at least one air channel of lung L. Although comparable high doses are used detrimental systemic effects may be prevented or considerably mitigated by preventing uncontrolled distribution of the medicament via the blood circuit, for instance to other organs, by using the closed transport volume mentioned above. This enables for instance to use stem cells in a stem cell therapy of lung L. It is prevented that the stem cells and/or other treatment substances are distributed to other organs via the blood circuit because the closed transport volume is used which enables removal of the stem cells in the auxiliary liquid outside of the body. This means that no treatment substance is introduces into the body via blood or another liquid which is transported in the closed transport volume TrV. A medicament/treatment substance is introduced or several medicaments/treatment substances are introduced only via inhalation or another transport method through at least one air channel of lung L.

Alternatively, during treatment of lung L using a medicament which flows through the blood vessels of lung L it is possible that the patient inhales or gets in another way via trachea Tr a medicament or treatment substance M in order to promote the treatment by the substance or medicament that flows within the fluid flow through the vessels of the lung L and through the tissue of the alveoli. The two treatment substances may be the same substances or may be different from each other.

In both cases, an inhalation device INH may be used that is coupled to a treatment substance delivery unit C2. Instead of inhalation other transport methods may be used for instance dropping a liquid into tube Tu which liquid comprises the treatment substance M. Air and/or oxygen O₂ may be sucked into inhalation device INH as a carrier for aerosols of the treatment substance which is inhaled. Not shown are optional one way valves, etc. which direct exhausted air into the environment or into a waste air system and which direct inhaled air to tube Tu.

Thus a method for treating the lung L of a body of a patient with a pharmaceutical or therapeutic treatment substance M is described. The method comprising:

transporting a treatment substance M through at least one substance transport channel of lung L to only a part of the alveoli of lung L in a selected region of lung L, the substance transport channel is preferably arranged within at least one air transport channel of lung L,

while treating and/or while transporting the treatment substance M through the at least one substance transport channel transporting a liquid, for instance blood or an auxiliary liquid, through the tissue of the part of the alveoli thereby using the transport volume TrV, and/or

while treating and/or while transporting the treatment substance M through the at least one substance transport channel transporting the liquid to the outside of a body of which lung L is part of.

The liquid may be cleaned outside of the body, for instance using a filter unit. The cleaned liquid may be used again in the closed transport volume TrV. Alternatively, the liquid may not be cleaned but replaced by fresh liquid. The fresh liquid (blood, auxiliary liquid, e.g. saline) may then be used in the closed transport volume. If only small amounts of liquid are used no cleaning or replacement may be necessary.

The treatment substance M may be transported to only one half lH, rH of lung L, to only one lobe Lo1 to Lo5 of lung L or to only a part of a lobe Lo1 to Lo5 of the lung L. Thus, treatment is extended only to an area of the lung which needs treatment. Healthy, areas of the lung may not be treated. This may allow that the untreated areas fulfill normal lung L functions during treatment of the selected area(s) of lung L. Normal lung functions are oxygen enhancement and carbon dioxide removal. If only small areas of the lung are treated no further lung L assist and/or heart H assist may be necessary. However, additional lung L assist and/or heart H assist may be used, especially if appropriate and/or necessary.

One of the following three variants or other variants may be used:

a) the treatment substance M may be transported only to a part or to all alveoli in one selected half lH, rH of lung L but not to non-selected alveoli in a non-selected half lH, rH of lung L and the liquid may be transported in an essentially closed or in a closed transport volume TrV at least through the tissue of the selected alveoli in the selected half lH, rH of lung L but not to alveoli in the non-selected half lH, rH of lung L, or

b) the treatment substance M may be transported only to alveoli in one selected lobe Lo1 to Lo5 of a selected half lH, rH of lung L but not to non-selected lobes Lo1 to Lo5 of the selected half lH, rH of lung L or only to alveoli in some selected lobes Lo1 to Lo5 of the selected half lH, rH of lung L but not to a non-selected lobe Lo1 to Lo5 in the selected half lH, rH of lung L and the liquid may be transported in an essentially closed or in closed transport volume TrV at least through the tissue of the alveoli in the selected lobe Lo1 to Lo5 or in the selected lobes Lo1 to Lo5 but not through the tissue of alveoli in a non-selected half lH, rH of lung L or in the non-selected lobes Lo1 to Lo5 of lung L, or

c) the treatment substance M may be transported only to a first part of the alveoli of a selected lobe Lo1 to Lo5 of a selected half lH, rH of lung L but not to a second part of alveoli of the selected lobe Lo1 to Lo5 and not to alveoli in a non-selected half lH, rH of lung L and the liquid may be transported in an essentially closed or in a closed transport volume TrV at least through the tissue of the first part of the alveoli in the selected half lH, rH of lung L but not through the tissue of alveoli in the non-selected half lH, rH of lung L of through the tissue of alveoli in the non-selected lobes Lo1 to Lo5 of the lung L.

A cannula L1b, L2b, etc. according to any one of examples mentioned above or a cannula system L1b, L2b; L1c, L2c, etc. according to any one of the examples mentioned above may be used for transporting the liquid. Furthermore, a method according to any one of the examples mentioned above may be used.

The treatment substance M may comprise stem cells of at least one kind of tissue in lung L. Alternatively or additionally the treatment substance M may comprise a medicament against lung L cancer or a medicament against a lung L infection, for instance against a virus infection, especially a SARS (Severe Acute Respiratory Syndrome) virus infection, more specifically SARS-CoV or SARS Covid 19.

The treatment substance M or at least a part of the treatment substance M may be removed from the liquid and the liquid may be transported back into the body, preferably back to the tissue.

A kit may be produced for defining at least one border of a transport volume for an in-vivo fluid transport. The kit or set may comprise:

a cannula L1b, L2b, etc. according to any one of the examples mentioned above, and/or

a cannula system L1b, L2b; L1c, L3c, etc. according to any one of the examples mentioned above, and:

at least one pump Pb for driving a fluid flow through the transport volume TrV, preferably a roller pump or a membrane pump or a centrifugal pump or a diagonal pump or an axial pump, etc.,

a transport device (tube Tu, inhalation device INH) comprising a treatment substance transport channel for transporting a treatment substance M, preferably through at least one air transport channel, for instance trachea Tr, bronchus Brla, Brlb, of lung L to only a part of the alveoli of lung L or to all alveoli of lung l, and

at least one of
a delivery unit C1b, C2 for the introduction of at least one treatment substance M into the fluid flow in order to guide treatment substance M and/or another treatment substance M into the transport volume TrV via the fluid flow, and/or p1 a removing device for removing the treatment substance M from the fluid flow.

The removing device may be preferably extracorporeal, for instance a filter unit as mentioned above.

The kit/set may be used for performing a method according to any one of the examples mentioned above.

In other words, a variant using medicament/treatment substance M (for instance stem cells) via trachea Tr into selective lobes Lo1 to Lo5 of lung L is provided. pIVLP™ (percutaneous in-vivo lung perfusion) may be enhanced by a circuit which comprises input via the bronchi to segments of lung L and output via the respective pulmonary vein (antegrade) or the respective pulmonary artery (retrograde). Outside of the body, filtering may take place and the liquid may be used again, for instance blood of the patient and/or blood of a blood donor. Thus, it is possible to isolate single lobes Lo1 to Lo5 which have a disease. These single lobes Lo1 to Lo5 or this single lobe Lo1 to Lo5 may be treated with high doses which may be filtered out again using iVLP (in-vivo lung perfusion), especially pIVLP™ (percutaneous in-vivo lung perfusion). This may prevent or mitigate systemic effects in other organs of the patient.

Furthermore, in other words, lung-directed high dose chemotherapy may evolve from a highly invasive open surgical procedure to a minimally invasive procedure known as percutaneous In-Vivo-Lung-Perfusion (pIVLP™). During pIVLP™, several cannulas may be placed percutaneously through interventional techniques. The lung L may be temporarily isolated from the body's circulatory system, during which time an infusion of the (for example) chemotherapeutic agent directly to the lung L may occur. The blood may be collected as it exits the lung L for filtration by proprietary filters prior to returning it to the patient. Returning the blood may be the best solution for some application. However, it is also possible to not return the used blood, for instance if the treatment substances or other by products cannot be removed completely or in an appropriate scale out of the fluid flow.

The following examples for pIVLP™ are described in more detail above:

1.) pIVLP™—low flow (for instance 0.5 liter per minute to 1 liter per minute), see FIG. 1B, (2× single lumen cannula, access jugular vein right and left, from LA to PA via pump and adsorber/filter, support drugs after adsorber),

2.) pIVLP™—medium flow (for instance 1.5 liter per minute to 3.5 liter per minute), see FIG. 1C, (1 double lumen cannula from RA to PA and single lumen cannula from LA, Y-connector before pump, after pump integrated oxygenator and adsorber/filter),

3.) pIVLP™—high flow (for instance 3.5 liter per minute to 5.0 liter per minute), see FIG. 2D, (1 double lumen cannula from RA to PA and single lumen cannula from LA, Y-connector before pump, after pump integrated oxygenator and adsorber/filter, then Y-connector to PA and femoral artery),

4a) pIVLP™—high double flow (for instance 5 liter per minute to 8.0 liter per minute), see FIG. 2E, (like 3. with 2 pumps)

4b) pIVLP™—high double flow with inhale, (like 4a +inhale via trachea), see FIG. 2E.

All variants may comprise or consist of the following method steps:

1. Isolation

First, an infusion cannula may be inserted into the left internal jugular vein IJV and guided so that the tip of the cannula is within the main pulmonary artery PA (or left (IPA)/right pulmonary artery (rPA), if only one lobe of lung L is treated) to deliver for instance the anticancer drug. Next, the isolation-aspiration cannula (for instance made by the company ReCO2lung) may be inserted into the right internal jugular vein IJV and guided trans-septal into the left atrium (LA), (or left (IPV)/right pulmonary vein (rPV), if only one lobe is treated). In the left atrium LA, one occlusion balloon of the isolation-aspiration catheter may be inflated to block the normal venous outflow of blood from the pulmonary vein PV to left atrium LA, thereby isolating lung L.

2. Infusion

High doses of chemotherapeutic agent may be delivered directly to lung L via the infusion cannula, saturating lung L and the tumor tissue.

3. Hemofiltration

The isolation-aspiration catheter may collect the blood as it exits lung L into the left atrium LA or in the pulmonary vein PV in the region of the inflated balloon and may then direct it out of the body 100. The blood then passes through a hemofiltration system (for instance a proprietary one made by the company ReCO2lung), which may reduce the concentration of chemotherapeutic agent. The filtered blood may now be returned to the patient's body 100 through a third cannula placed in the internal jugular vein IJV, or return it in the circuit of the IVLP (in-vivo lung perfusion) or pIVLP™ (percutaneous in-vivo lung perfusion).

Furthermore, the following may apply, especially for a treatment of the lung L:

Drugs/substances for lung diseases that may be used: Doxorubicin (may be a registered trademark), 5-flurodeoxyuridine (may be a registered trademark), i.e. FUDR (may be a registered trademark), tumor necrosis factor alpha (may be a registered trademark), i.e. TNF-α (may be a registered trademark), paclitaxel (may be a registered trademark), melphalan (may be a registered trademark), gemcitabine (may be a registered trademark), cisplatin (may be a registered trademark) and combined use have all been used for pulmonary metastases. Further substances that may be used separate or in combination with other substances are: carboplatin (may be a registered trademark), bleomycin (may be a registered trademark), mitomycin (may be a registered trademark), especially mitomycin C (may be a registered trademark), etc. All drugs/substances may be administered also in liposomal-encapsulated, especially gemcitabine, cisplatin and carboplatin. A liposome may be a spherical vesicle comprising or having at least one lipid bilayer.

pIVLP™ or the proposed invention may allow the lung L to be preferentially perfused with high doses of chemotherapy to the tumor, avoiding the dose-limiting effects of systemic toxicity while providing targeted therapy for both macroscopic disease and microscopic disease.

pIVLP™ or the proposed invention may be performed retrograde or antegrade. The proposed benefit behind retrograde perfusion is that, by the perfusion through the pulmonary vein PV, collaterals between the pulmonary and bronchial venous systems may be exploited to deliver drugs to the metastatic lesions. In case of pulmonary embolism the proposed benefit behind retrograde perfusion is that, a retrograde flow and pressure will flush thrombus even from small capillaries out to the PA.

pIVLP™ or the proposed invention may be performed even with perfusion pressures lower than 25 mm Hg to avoid producing functional and morphologic damage to the perfused lung.

pIVLP™ or the proposed invention may be performed with hyperthermic conditions, using enhanced cytotoxic effects at higher temperatures. There may be a combination with the usage of chemical treatment substance (chemotherapy) and/or radiation methods as mentioned above, especially radioactive radiation, etc. Combination may refer to at the same time, i.e. simultaneously, within a period that is less than 24 hours, less than one week or less than one month.

pIVLP™ or the proposed invention may minimize the impact of active drug loss from renal metabolism of the drugs.

After pIVLP™ or the proposed invention is complete, the lung is with varying lengths of washout flushed with flushing fluid, for instance with normothermic saline, Voluven (may be a registered trademark), lactated Ringer's solution (may be a registered trademark), or Hespan (may be a registered trademark). After complete washout, the pulmonary artery PA and pulmonary vein PV cannulas may be removed.

pIVLP™ or the proposed invention may be performed multi times, even as staged procedure over several week, or months, especially advantageous for patients needing repeated therapies.

pIVLP™ or the proposed invention may be performed as bi-lateral or single-lateral or as only partially to a part of an organ that is less than half, less than a third or less than a quarter of the organ.

The treatment fluid (within pulmonary veins and/or pulmonary arteries and/or within air channels of the lung) flow may be heated in order to improve the uptake of medicaments/treatment substances by the tissue of the organ and/or by the cells of the organ. If the treatment fluid comprises blood or a high percentage of blood or blood components the heating temperature may be for instance in the range between 39.0 and 44.0° C. (degrees of Celsius), preferably to between 40.0 and 42.5° C.

However, due to the isolation of the transport volume from the body fluid and/or due to the local treatment even higher temperatures may be used, especially if the fluid flow through the transport volume does not contain or comprise blood or blood components or only a lower percentage of blood per volume. Therefore, also temperatures above 42.5° C. may be used, for instance above normal blood temperature, above 43° C., above 44° C., above 45° C. or even above 50° C. This may improve the uptake of medicaments/treatment substances further.

The term “normal body temperature (also known as normothermia or euthermia)”, as used herein, may refer to the typical temperature found in an individual. In humans, the normal body temperature is 37° C. This value is, however, only an average. The normal body temperature may be slightly higher or lower. A number of factors can influence the body temperature, including age, sex, time of day, and activity level. In babies and children, for example, the average body temperature ranges from 36.6° C. to 37.2° C. Among adults, the average body temperature ranges from 36.1° C. to 37.2° C. The normal human body temperature range is, thus, typically stated as being between 36.1° C. and 37.5° C., e.g. 36.1, 36.2, 36.3, 36.4, 36.5, 36.6, 36.7, 36.8, 36.9, 37.0 37.1, 37.2, 37.3, 37.4, or 37.5° C., in humans.

Furthermore, it is possible to use in all embodiments that are mentioned above an inner surface of the lumen portion and/or inner lumen that comprises a spirally and/or helically surface structure. The spirally and/or helically surface structure may have the effect that the fluid flow within the cannula is rotated as it moves through the cannula. Turbulences may be reduced thereby and/or it may be possible to reach much higher flow rates compared to cannulas that have a smooth inner surface, i.e. that do not have spirally and/or helical surface structures on their inner surfaces. However, it is of course possible to use cannulas without a spirally and/or helical surface features, if for instance lower flow rates are necessary. The spirally turned flow and/or the rotated flow may prevent clotting of blood cells if the fluid flow comprises blood, especially in slow flow rate conditions. However, there may also be advantages if the fluid flow does not contain blood. The spiral flow may be a laminar spiral flow.

There may be an embodiment in which a single lumen cannula or a dual or multi lumen cannula is used (fixed or non-fixed) wherein the single lumen cannula or the inner cannula of the dual or multi lumen cannula may have a split tip. Each distal tip of the split tip cannula may be associated with or may be carry an expandable arrangement, for instance a balloon or a cage, especially with a cage that carries a membrane. The distal parts of the split tip cannula may be inserted into the left pulmonary veins whereby the right pulmonary veins may be left open. Alternatively, the distal parts of the split tip cannula may be inserted into the right pulmonary veins whereby the left pulmonary veins may be left open.

Furthermore, there may be an embodiment in which a single lumen cannula or a dual cannula is used (fixed or non-fixed) wherein the single lumen cannula or the inner cannula of the dual lumen or multi lumen cannula may have a cage arrangement on its distal end. The cage arrangement may carry a membrane. The membrane may define an opening that faces laterally. If the cannula is inserted into the body, the opening of the membrane may face laterally in the direction of both right pulmonary veins. Both left pulmonary veins may remain open, i.e. blood may pass the outside of the membrane thus not entering the cannula that carries the cage arrangement with the membrane.

The following feature combinations may be relevant:

a) All embodiments that relate to a cage arrangement or to another expandable arrangement may be used to reach a stable and/or secure positioning or fixation of the cannula in the chambers of the heart (left atrium LA, right atrium RA, left ventricle LV, right ventricle RV) or vessels of the blood circuit. The cage arrangement or another expandable arrangement may allow a better design of the cannula, especially of the distal tip, for instance only one end-hole may be used instead of multi-hole distal parts. This may result in better or optimal flow characteristics, for instance less shear stress and/or less turbulences less shear rates and/or less recirculation and/or more homogeneous velocity profile, etc.

The cannula may comprise at least one end hole or a single end-hole through which at least 25 volume percent, at least 50 volume percent, at least 75 volume percent or at least 90 volume percent or all of the flow flows into or out of the cannula, all volumes measured for instance for 3.5 1 per minute or for 5 1 per minute. The given volume percent of flow through the at least one end-hole may be measured for instance for a flow through the cannula of 3.5 1 per minute or for 5 1 per minute. Thus, a significant portion of the flow flows through the at least one end hole. Additionally, the cannula may be a “tip-less” cannula which extends not or only at most 3 mm (millimeter) within the inner volume of the cage in the expanded state. Thus, the fixation of the cage may extend only up to the distal end of the cannula or up to a location which is equal to 3 mm or at most 3 mm away from the distal end of the cannula.

In another embodiment at least one side hole may be present in the cannula in which at least 25 volume percent, at least 50 volume percent, at least 75 volume percent or at least 90 volume percent or all of the flow flows into or out of at least one end-hole of the cannula and/or in the case of a tip-less cannula having an axial oriented inlet or outlet. The at least one side hole may have at least one other function in addition to the function of flow transport into or out of the cannula. The flow transport through the at least one side-hole may be equal to or less than 50 volume percent of the overall flow through the cannula or equal to or less than 25 volume percent of the overall flow through the cannula. The given volume percent of flow through the at least one side hole may be measured for instance for a flow through the cannula of 3.5 1 per minute or for 5 1 per minute.

b) All embodiments may be used with fix or non-fixed dual lumen cannulas or multi lumen cannulas. If non-fixed (may be inserted into each other) cannulas are used, it is easier to position or implant the cannulas stepwise along a curved introduction path because less friction may be involved, especially at the puncture site, and the insertion may be less traumatic.

c) All embodiments may be used for endovascular access and for in-vivo lung isolation which may allow an isolated perfusion of lung L or of parts of lung L, for instance in a closed circuit of fluid flow that may be isolated from the body blood circuit.

d) If within the cage a cannula tip is used which has side-holes it is possible to have at least one end-hole or to have no end-holes.

The combination of at least two arbitrarily selected or of all feature combinations a), b) and c) may give the best result.

Moreover, the cannula, for instance the delivery cannula, may be inserted endovascular jugular and may be punctured from superior vena cava SVC or from right atrium RA transcaval to ascending aorta aAO. Alternatively, the cannula, for instance the delivery cannula, may be inserted endovascular femoral through inferior vena cava IVC into the right atrium RA and may be punctured from superior vena cava SVC or from right atrium RA transcaval to ascending aorta aAO.

Moreover, the delivery cannula or the drainage cannula may be inserted endovascular jugular and may be punctured from superior vena cava SVC or from right atrium RA transcaval to the main pulmonary artery PA or to the right pulmonary artery rPA or in special cases to the left pulmonary artery IPA. Alternatively, the delivery cannula or the drainage cannula may be inserted endovascular femoral through inferior vena cava IVC into the right atrium RA and may be punctured from superior vena cava SVC or from right atrium RA transcaval to the main pulmonary artery PA or to the right pulmonary artery rPA or in special cases to the left pulmonary artery IPA.

In all embodiments with a cage arrangement it is also possible to use another material than a metal, for instance a natural and/or biological material, especially cellulose, for instance cellulose that is treated to increase the hardness. Compatibility with body 100 and/or with blood may be improved thereby.

In all embodiments one of the following methods may be used to bring or guide a guide wire and/or a catheter around or along the acute angle within the left ventricle LV. At least one snare may be used to catch the catheter and/or the guide wire in the left ventricle LV. The methods may be performed independent whether there is jugular access or a femoral access or another access for the catheter and/or the guide wire.

Variant A (catching the catheter with the snare):

1) Introducing a catheter through the right atrium RA, the atrial septum AS (a puncturing step may be performed earlier or using the catheter, e.g. using a needle and/or RF (radio frequency) tip/wire within the catheter). The catheter may be introduced further through the hole (puncture) in the atrial septum AS through left atrium LA, through mitral valve MV into the left ventricle LV.

2) Introducing a snare from descending aorta AO through aortic valve AV into left ventricle LV. This step may be performed also before step 1.

3) Catching the catheter in the left ventricle LV using the snare.

4) Pulling the snare and the distal end of the catheter therewith to the aorta AO.

5) Introducing a guide wire through the catheter.

6) Forwarding the guide wire out of the distal end of the catheter. Slight loosening of the snare may be optionally performed thereby.

7) As the guide wire is already within the snare, pull back the snare to a region in which only the guide wire is located but not the catheter.

8) Fix the guide wire using the snare, e.g. contract the snare and/or tighten the snare.

9) Optional, externalizing for instance the distal end of the guide wire out of the body. This step is optionally, because the proximal end of the snare is already outside of the body. 10) Remove catheter, e.g. pull back the catheter. 11) Introduce cannula using the guide wire, e.g. pushing the cannula along and/or over the guide wire until it is on its final place.

Variant B (catching the guide wire with the snare):

1) Introducing a catheter through the right atrium RA, through the atrial septum AS (a puncturing step may be performed earlier or through catheter, use needle and/or RF (radio frequency) tip/wire). Introducing the catheter further through left atrium LA, mitral valve MV into the left ventricle LV.

2) Introducing a guide wire through the catheter until the distal end of the guide wire comes out of the distal end of the catheter within the left ventricle LV. The RF wire may be used also as a guide wire.

3) Introducing a snare from descending aorta AO through aortic valve AV into left ventricle LV. This step may be performed before step 1 and/or before step 2.

3) Catching the distal end of the guide wire in the left ventricle LV using the snare.

4) Fixation of the guide wire using the snare.

5) Pulling the snare and the distal end of the guide wire therewith to the aorta AO.

6) Optional, externalizing guide wire by pulling it out of the body using the snare. This step is optional as the snare is already outside of the body from where it has been introduced.

7) Remove catheter, e.g. by pulling it back along the guide wire.

8) Introduce cannula over/along the guide wire until it is on place.

The following method may also be used in all corresponding embodiments for introducing a cannula jugularly transseptally:

1) Introduce a first snare into an internal jugular vein IJV, for instance into the right jugular vein RJV or into the left jugular vein LJV.

2) Advancing the first snare to inferior vena cava IVC.

3) Introducing a catheter into a common femoral vein CFV (left or right).

4) Advancing the catheter through the first snare into an inferior vena cava IVC.

5) Advancing the catheter through the first snare into the vena cava VC in an antegrade fashion.

6) Advancing the catheter through the first snare into the right atrium RA in an antegrade fashion.

7) Advancing the catheter through the first snare and from the right atrium RA transseptally through the atrial septum into the left atrium LA in an antegrade fashion. Puncturing of atrial septum may have been performed earlier. Alternatively, the catheter is used to puncture the atrial septum, for instance using a needle or using a RF (radio frequency) wire/tip which is introduced trough the catheter.

8) Advancing the catheter through the first snare and advancing the catheter across the mitral valve MV and into the left ventricular outflow tract, e.g. the left ventricle LV.

9) Advancing a second snare in the ascending aorta AO catching and snaring a distal portion of the catheter (Variant A) within the left ventricle LV. The second snare may optionally be introduced through an artery, which may include, but is not limited to, a radial artery, a brachial artery, an axillary artery, a subclavian artery, a carotid artery, or common femoral artery, and advanced retrograde into the aorta AO and into the left ventricle LV. The second snare may be already introduced before the catheter is introduced. Alternatively, a guide wire may be inserted into the catheter until a distal end of the guide wire comes out of a distal opening of the catheter. This distal end of the guide wire is then caught and snared within the left ventricle (Variant B)

10) Pulling the catheter (Variant A) or the guide wire (Variant B) into the aorta AO in an antegrade fashion using the second snare.

11) In variant A, advancing a guide wire through the catheter and through the first snare in antegrade fashion to the ascending aorta AO and through the second snare. Snaring the distal end of the guide wire in variant A but not the catheter.

12) In both variants A and B remove the catheter with the guide wire remaining in the heart H and through the first snare after the catheter is removed.

13) Externalizing a proximal portion of the guide wire from femoral vein, through inferior vena cava IVC, through inferior vena cava SVC, into the internal jugular vein IJV and then out of the internal jugular vein IJV using the first snare, for instance left jugular vein LJV or right jugular vein RJV. In some embodiments the snare may externalize a different portion of the guide wire, for instance an intermediate portion.

14) Advancing a cannula using the guide wire and/or along and/or over the guide wire from the internal jugular vein IJV. The cannula may be any of the cannulas described in this specification or known in the art. Especially, an outer cannula may be advanced over the guide wire from the internal jugular vein IJV. An inner cannula may optionally be advanced through a port proximal of the distal end of the outer cannula. The inner cannula and the outer cannula may be positioned as described in this description, or if a single multi-lumen cannula is used, it may be positioned in a similar manner

15) Optionally, a distal portion of the guide wire may be externalized out of the body through the artery. This step is optional because the second snare is already externalized and may form a secure anchor for the distal portion of the guide wire.

Subclavian arteries/veins or other arteries/veins may be used for introducing the snare(s) because the snares require smaller diameters, e.g. less than 10 French (1 French equal to ⅓ mm (millimeter)) or less than 8 French, e.g. more than 3 French, compared to the diameters of the cannula(s).

In the following details of a method for puncturing transseptally through the atrial septum AS of the heart H are provided. However, other methods may be used as well, for instance using a needle.

A catheter and/or a wire may be used which has a distal tip which can be heated, for instance using RF (radio frequency) energy, alternating current (ac), direct current (dc) etc. Thus, e.g. a hole may be burned into the septum, e.g. the atrial septum AS, during puncturing, for instance using temperatures above 100° C. (degrees Celsius) or above 200° C., less than 1000° C. for instance.

The RF (radio frequency) may be in the range of 100 kHz (kilohertz) to 1 MHz (Megahertz) or in the range of 300 kHz to 600 kHz, for instance around 500 MHz, i.e. in the range of 450 kHz to 550 kHz, e.g. 468 kHz.

The power of the radio frequency energy may have a maximum of 50 Watt. A power range of 5 W (watt) to 100 W may be used, for instance a range of 10 W to 50 W.

A sinus current/voltage may be used for the RF. The sinus current/voltage may be continuous. Alternatively, a pulsed sinus current/voltage may be used for the RF.

All parameters or some of the parameters of the RF equipment may be adjustable by an operator who performs the puncturing, for instance dependent on the specifics of the septum, e.g. normal septum, fibrotic septum, aneurysmal septum, etc. Preferably, the power may be adjustable.

A solution of Baylis Medical (may be a trademark), Montreal, Canada may be used, for instance NRG® trans-septal needle or Supra Cross® RF Wire technology. RF generator of type RFP-100A or a further development of this model may be used. This RF generator uses for example a frequency of 468 kHz (kilohertz).

A single puncture of the septum may be performed from a jugular access or from a femoral access or from another appropriate access using the RF energy Smaller angles may be possible for the catheter if for instance compared with a needle.

Alternatively, the RF method may be used also if two separate punctures are made in the septum. However, usage of needles is possible as well. One of the punctures using the RF method may be made through left jugular vein LJV and the other puncture of the atrial septum AS may be made through the right jugular vein RJV.

It is possible to introduce both guide wires first through the atrial septum AS. Preferably, separate holes are used for each of the guide wires. Guide wire(s) may be used which include an RF tip. Alternatively, the wire(s) having the RF tip may be pulled back and a further wire may be introduced through the catheter.

Only after both guide wires are in place, both cannulas may be introduced using a respective one of the guide wires.

Alternatively, the first puncture may be performed using RF energy or a needle. Thereafter, the first cannula for blood transfer is inserted using the first guide wire. After insertion of the first cannula, the second puncture may be made. A second guide wire or the first guide wire may be used to introduce the second cannula.

Puncturing of the atrial septum may be assisted by at least one medical imaging method, preferably by at least two medical imaging methods.

US (ultra-sonic) echo imaging may be used to visualize the movement of heart H and the location of the valves of heart H. No dangerous radiation may result from ultra-sonic imaging. An ultra-sonic transmitter may be introduced for instance via the esophagus, e.g. trans esophagus echo (TEE) may be used.

X-ray radiation preferably in combination with fluorescence (fluoroscopy), may be used in order to visualize the location of catheters (comprising for instance at least one X-ray marker, or the devises are usually radiopaque) and/or the location of guide wire(s), snares etc.

Thus, transseptal puncturing or puncturing of other tissue may be guided by TEE and by fluoroscopy or by other imaging methods. At least two different image generating methods may be used.

In all embodiments mentioned above, it is also possible to use a soft guide wire and a stiffer guide wire which does not bend so easy if compared with the soft guide wire. The following steps may be performed, preferably in combination with snaring:

1) Introduce a soft guide wire.

2) Introduce catheter using the soft wire as a guide.

3) Optionally, remove soft wire, for instance by pulling back the soft wire out of the catheter.

4) Introduce stiffer guide wire into the catheter, e.g. there may be a change of wire from soft wire to the stiffer wire.

The catheter may be removed, e.g. pulled back. Thereafter, the stiffer wire may be used to introduce a cannula or cannulas.

Although embodiments of the present disclosure and their advantages have been described in detail, it should be understood that various changes, substitutions and alterations may be made therein without departing from the spirit and scope of the disclosure as defined by the appended claims. For example, it will be readily understood by those skilled in the art that many of the features, functions, processes and methods described herein may be varied while remaining within the scope of the present disclosure. Moreover, the scope of the present application is not intended to be limited to the particular embodiments of the system, process, manufacture, method or steps described in the present disclosure. As one of ordinary skill in the art will readily appreciate from the disclosure of the present disclosure systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps. Further, it is possible to combine embodiments mentioned in the first part of the description with examples of the second part of the description which relates to FIGS. 1 to 19. Moreover, it is possible to combine embodiments mentioned in the first part of the Figures, i.e. FIGS. 1A to 2E, with examples of the second part of the Figures, which relates to FIGS. 3 to 12. The embodiments shown in FIGS. 13 to 18 may also be combined with the embodiments shown in one of the FIGS. 1A to 12 or mentioned in the first part of the description. Moreover, the embodiment of FIG. 19 may be combined with the embodiment of any other of the FIGS. 1A to FIG. 18, e.g. especially with the usage of a closed or almost/essentially closed transport volume TrV within the pulmonary veins PV and/or pulmonary arteries PA.

1. Endovascular cannula (L1b, L2b) for defining a border of a transport volume (TrV) for an in-vivo fluid transport within the transport volume (TrV),
the cannula (L1b, L2b) comprising:
a lumen portion (LP) that extends between a proximal end of the cannula (L1b, L2b) and a distal end of the cannula (L1b, L2b), the lumen portion (LP) defining an inner lumen, and
an expandable arrangement that has a non-expanded state and an expanded state,
wherein the expandable arrangement can be switched from the non-expanded state to the expanded state,
wherein in the expanded state the expandable arrangement is adapted to define at least one border of the transport volume (TrV),
wherein the border is configured to separate the transport volume (TrV) from a body fluid circuit (BC).

2. Cannula (L1b, L2b) according to claim 1, wherein the inner lumen is arranged and configured to be in fluid communication with the transport volume (TrV) in the expanded state of the expandable arrangement, and
wherein the lumen portion (LP) is configured to be guided through the body fluid circuit (BC).

3. Cannula (L1b, L2b) according to claim 1 or 2, wherein the lumen portion (LP) comprises two separate distal parts (L18a, L18b), a first distal part and a second distal part, that extend away from a bifurcation region (Bi) of the lumen portion (LP, L18) in the distal direction,
wherein the expandable arrangement is a first expandable arrangement, and
wherein the first expandable arrangement is associated with the first distal part.

4. Cannula (L1b, L2b) according to one of the claims 1 to 3, wherein the at least one expandable arrangement is inflatable,
wherein the expandable arrangement comprises a balloon (Ba) that is inflatable using a liquid fluid or a gaseous fluid, and/or
wherein the balloon (Ba) has a length of at least 2 mm, of at least 5 mm, at least 10 mm, at least 15 mm or of at least 20 mm,
wherein the cannula comprises a further lumen (CH1, CH2) for guiding a fluid into and/or out of the balloon (Ba), and wherein the further lumen is separated from the inner lumen.

5. Cannula (L1b, L2b) according to one of the claims 1 to 3, wherein the expandable arrangement comprises a cage arrangement (916, 946) that comprises a plurality of wires (1612), and
wherein the expandable arrangement comprises a membrane (919, 949) that is connected to at least two of the wires (1612) of the plurality of wires (1612).

6. Cannula (L1b, L2b) according to one of the preceding claims, wherein the lumen portion (LP) is adapted to guide an introducer member (2114),
and wherein the expandable arrangement comprises a contact area that is adapted to have mechanical contact with the introducer member (2114),
wherein, in the expanded state and/or the non-expanded state, the contact area overlaps with an opening of the lumen portion (LP) as seen in a top view onto the opening along a longitudinal axis defined by the lumen portion (LP),
and wherein the expandable arrangement is configured such that it changes from the non-expanded state to the expanded state if the introducer member (2114) is moved away from the contact area.

7. Cannula (L1b, L2b) according to one of the preceding claims, wherein the cannula (L1b, L2b) has an insertable length that is more than 25 cm or more than 30 cm, and/or
wherein a maximal width of an opening at the distal part of the lumen portion is less than 15 mm, less than 14 mm, less than 13 mm, less than 12 mm, less than 11 mm, less than 10 mm and/or more than 5 mm, more than 6 mm, more than 7 mm or more than 8 mm, and/or
wherein the at least one expandable arrangement has a maximum diameter or width in the expanded state that allows fixation of the at least one expandable arrangement and/or placement within the main pulmonary artery (PA), within the left pulmonary artery (IPA), within the right pulmonary artery (rPA), within a left pulmonary vein (IPV) and or within a right pulmonary vein (rPV) of the subject.

8. Cannula (L1b, L2b) according to one of the preceding claims, wherein the at least one distal part of the lumen portion (LP) comprises only one end-hole (EH) that is arranged on the longitudinal axis of the at least one distal part of the lumen portion (LP).

9. Cannula (L1b, L2b) according to one of the preceding claims, wherein the cannula (L1b, L2b) comprises at least one pre-formed bend (K) that defines an angle within the range of 90 degrees to 170 degrees, and/or
wherein the cannula (L1b, L2b) is adapted for endovascular insertion into the lung (L) of a body (100) of a subject.

10. Cannula system (L1b, L2b; L1c, L2c ) for defining at least one border of a transport volume (TrV), especially for an in-vivo fluid transport and/or in-vivo fluid isolation, comprising:
a cannula (L1b, L2b) according to one of the claims 1 to 9 that is a first cannula (L1b), wherein the lumen portion is a first lumen portion (LP) and wherein the expandable arrangement is a first expandable arrangement, and at least one of:
a second cannula (L3c) and/or a further cannula (L2b).

11. Cannula system (L1c, L3c) according to claim 10, wherein the second cannula (L3c) comprises a second lumen portion that extends between a proximal end of the second cannula and a distal end of the second cannula (L3c), the second lumen portion defining a second inner lumen, and
wherein a part of the first cannula (L1c) is arranged or is configured to be arranged within the second lumen portion,
wherein the distal end of the first cannula (L1c) is arranged or is configured to be arranged outside of the second lumen portion, and/or
wherein the first lumen portion is insertable into the second lumen portion such that the distal end of the first cannula (L1c) is arranged outside of the second lumen portion.

12. Cannula system (L1c, L3c) according to claim 11, wherein the expandable arrangement is a first expandable arrangement, and wherein
a second expandable arrangement is arranged and/or mounted on a distal part of the second lumen portion,
wherein the second expandable arrangement has a non-expanded state and an expanded state,
wherein the second expandable arrangement encompasses or defines in the expanded state a volume that is greater than the volume that is encompassed or defined by the second expandable arrangement in the non-expanded state.

13. Cannula system (L1c, L3c) according to claim 12, wherein the second expandable arrangement comprises a cage arrangement that comprises a plurality of wires (1612),
and wherein preferably the cage arrangement comprises no membrane.

14. Cannula system (L1b, L2b; L1c, L2c) according to one of the claims 11 to 13, wherein the second cannula (L3c) comprises a first group of holes (1098) and a second group of holes (1090),
wherein the smallest distance between any arbitrarily selected pair of holes having one hole in the first group (1098) and another hole in the second group (1090) is more than the minimum distance between any arbitrarily selected pair of holes having both holes within the first group (1098) or having both holes within the second group (1090) of holes,
wherein the first group (1098) of holes is arranged more distally than the second group.

15. Cannula system (L1b, L2b) according to one of the claims 11 to 14, comprising a first introducer member (2114) that is adapted to be introduced within the first cannula (L1c),
wherein preferably the first expandable arrangement is in the non-expanded state if the first introducer member is in contact with a contact portion of the first expandable arrangement and wherein the first expandable arrangement is in the expanded state if the first introducer member (2114) not inserted within the first lumen portion (LP).

16. Cannula system (L1b, L2b) according to one of the claims 10 to 15,
wherein the further cannula (L2b) is configured to be used independently of the first cannula (L1c),
wherein the further cannula (L2b) comprises a further lumen portion that extends from a proximal part of the further cannula (L2b) to at least one distal part of the further cannula (L2b), and
at least one further expandable arrangement associated with the at least one distal part of the further lumen portion.

17. Cannula system (L1b, L2b) according to claim 16, wherein the further cannula (L2b) has an insertable length that is more than 15 cm, 20 cm or more than 25 cm, and/or
wherein a maximal width of an opening at the distal part of the lumen portion is less than 15 mm, less than 14 mm, less than 13 mm, less than 12 mm, less than 11 mm, less than 10 mm less than 9 mm, less than 8 mm, less than 7 mm, less than 6 mm, less than 5 mm and/or more than 5 mm, more than 6 mm, more than 7 mm or more than 8 mm, and/or
wherein the further expandable arrangement has a maximum diameter or width in the expanded state that allows fixation of the further expandable arrangement and/or placement within the main pulmonary artery (PA), within the left pulmonary artery (IPA), within the right pulmonary artery (rPA), within a left pulmonary vein (IPV) and/or within a right pulmonary vein (rPV) of the subject,
and/or wherein the further lumen portion comprises two separate distal parts (L18a, L18b) that extend away from a bifurcation region (Bi) of the further lumen portion in the distal direction, preferably by at least 1 cm, at least 1.5 cm or at least 2 cm respectively,
wherein a first distal part (L18a) of the first lumen portion is associated with a first further expandable arrangement of the at least one further expandable arrangement distal from the bifurcation region (Bi) and/or wherein a second distal part (L18b) of the lumen portion is associated with a second further expandable arrangement of the at least one further expandable arrangement distal from the bifurcation region (Bi).

18. Cannula system (L1b, L2b) according to claim 16 or 17, comprising an inner cannula that is adapted for the delivery of oxygenated blood into an artery of the subject during the in-vivo fluid transport through the lung (L) of the subject, or comprising a single lumen cannula (L4d, L4e) that is adapted for the delivery of oxygenated blood into an artery of the subject.

19. Kit for defining at least one border of a transport volume (TrV) for an in-vivo fluid transport, comprising:
a cannula (L1b, L2b) according to one of the claims 1 to 9, and/or
a cannula system (L1b, L2b; L1c, L3c) according to one of the claims 10 to 18, and:
at least one pump (Pb) for driving a fluid flow through the transport volume (TrV), preferably a roller pump or a membrane pump or a centrifugal pump or a diagonal pump or an axial pump, and/or
a delivery unit (C1b) for the introduction of at least one treatment substance into the fluid flow in order to guide the treatment substance into the transport volume (TrV) via the fluid flow.

20. Kit according to claim 19, wherein the at least one treatment substance is a substance for treating cancer or an infectious disease and wherein the treatment substance is not part of the kit.

21. Kit according to claim 19 or 20, wherein only one pump (Pb) is comprised within the kit and wherein the pump (Pb) is adapted to create a flow through the transport volume (TrV) within a range of 0.5 liter per minute to 1.0 liter per minute, or
wherein only one pump (Pc) is comprised within the kit and wherein the pump (Pc) is adapted to create a flow through the transport volume (TrV) within a range of 1.5 liters per minute to 3.5 liters per minute, or
wherein only one pump (Pd) is comprised within the kit and wherein the pump (Pd) is adapted to create an overall flow through the transport volume (TrV) and through a further part of the body (100) of a subject, preferably comprising oxygenated blood, within a range of 3.5 liters per minute to 5.0 liters per minute, or
wherein at least two pumps (Pe1, Pe2) are comprised within the kit and wherein the pumps (Pe1, Pe2) are adapted to create an overall flow through the transport volume (TrV) and through a further part of the body (100) of a subject, preferably comprising oxygenated blood, within a range of 5 liters per minute to 8 liters per minute.

22. Kit according to one of the claims 19 to 21, comprising at least one, at least two, at least three, at least four, at least five arbitrarily selected or all of the following units or elements:
an oxygenator unit (OXYb) for oxygenating blood and/or the fluid flow, and/or
a carbon dioxide removal unit for removing carbon dioxide from blood and/or the fluid flow, and/or
an inhalation unit (INH) that enables the inhalation of a treatment substance by the subject, wherein the inhalation unit is configured to enable inhalation of the treatment substance while driving the fluid flow through the transport volume (TrV), and/or
at least one Y-connector for connecting the cannula (L1b, L2b) to at least one other unit or element of the kit, and/or,
at least one unit for controlling a respective pump (Pb) of the kit in pulsatile pumping mode or in continuous flow mode or that is adapted to switch between pulsatile mode and continuous mode.

23. Kit according to claim 22, wherein the treatment substance that is applied by inhalation is a substance for treating cancer or an infectious disease.

24. Method for an in-vivo fluid transport within a transport volume (TrV), comprising:
inserting a cannula (L1b) endovascularly into a body fluid circuit (BC),
wherein the cannula (L1b) comprises a lumen portion (LP) that extends between a proximal end of the cannula (L1b) and a distal end of the cannula (L1b), the lumen portion (LP) defining an inner lumen, forming at least one border within the body fluid circuit (BC) to delimit the transport volume (TrV) from the body fluid circuit (BC),
wherein the border is arranged within a conduit (V) of the body fluid circuit (BC) and wherein the border blocks a body fluid flow within the conduit (V) of the body fluid circuit (BC) along the cannula (L1b) beyond the border, and
after forming the border, guiding a fluid flow through the cannula (L1b) into the transport volume (TrV) wherein the at least one border is arranged to separate the transport volume (TrV) from the body fluid circuit (BC).

25. Method according to claim 24, wherein at least a part of the transport volume (TrV) is arranged within the lung (L) or within a single lobe of lung (L) or within a part of a single lobe of lung, preferably within the tissue of the alveoli of the lung (L) or of a single lobe of lung (L) or of a part of a single lobe of lung.

26. Method according to claim 25, wherein the transport volume (TrV) extends only within one lobe of the lung (L) of a subject or only within a part of one lobe of the lung (L),
and wherein preferably right heart (H) support and/or lung (L) support are not performed during guiding the fluid flow through the transport volume (TrV).

27. Method according to claim 25, wherein the transport volume (TrV) is a first transport volume, wherein during a first duration the first transport volume (TrV) is used to guide the fluid flow,
wherein the location of the cannula and/or a further cannula is changed in order to treat another region of the lung (L) or of a single lobe of lung (L) or of another part of a single lobe of lung, and
wherein during a second duration that follows after the first duration and after changing the location of the cannula and/or of the further cannula a second transport volume that is different from the first transport volume (TrV) is used to guide the fluid flow.

28. Method according to claim 27, wherein the first transport lumen (TrV) is arranged in a first lobe of the lung (L) and wherein the second transport volume is arranged in a second lobe of the lung (L) that is different from the first lobe of the lung (L).

29. Method according to claim 27 or 28, wherein the lumen portion (LP) comprises a first distal part (L18a) and a second distal part (L18b) which extend away from a bifurcation region (Bi) of the lumen portion (LP, L18) into the distal direction, preferably by at least 1 cm, at least 1.5 cm or at least 2 cm respectively,
wherein the border is a first border that is arranged distally from the bifurcation region (Bi) and is associated with the first distal part (L18a),
wherein the conduit (V) is a first conduit (V),
and wherein the second distal part (L18b) is arranged within a second conduit of the body fluid circuit (BC) and wherein a second border blocks a body fluid flow within the second conduit of the body fluid circuit (BC) along the second distal part (L18b) beyond the second border, and
wherein second border is arranged distally from the bifurcation region (Bi) and is associated with the second distal part (L18b),
wherein the first distal part (L18a) is arranged within a first one of the left pulmonary veins (IPV) and the second distal part (L18b) is arranged within a second one of the left pulmonary veins (IPV) or wherein alternatively the first distal part (L18a) is arranged within a first one of the right pulmonary veins (rPV) and the second distal part (L18b) is arranged within a second one of the right pulmonary veins (PV).

30. Method according to one of the claims 26 to 29, a) wherein the transport volume (TrV) is arranged in one lobe of the lung (L) and wherein only this lobe is treated only one time by guiding a treatment substance into the transport volume (TrV) through the cannula (L1b), and wherein the other lobe of the lung (L) is not treated, or
b) wherein the transport volume (TrV) is arranged in one lobe of the lung (L) and wherein only this lobe is treated several times within several endovascular treatment sessions by guiding treatment substance into the transport volume (TrV), and wherein the other lobe of the lung (L) is not treated, and wherein preferably the duration between both treatment sessions is at least one month, at least one week or at least 24 hours.
c) wherein the transport volume (TrV) is arranged in one lobe of the lung (L) and wherein within one treatment session only one lobe is treated by guiding a treatment substance into the transport volume (TrV) and wherein the other lobe of the lung (L) is treated in a different treatment session, and wherein preferably the duration between both treatment sessions is at least one month, at least one week or at least 24 hours.

31. Method according to claim 25, wherein both lobes of the lung (L) are treated simultaneously by guiding a treatment substance into the transport volume (TrV) through the cannula (L1b), and
wherein right heart (H) support and/or lung (L) support is performed during guiding the fluid flow through the transport volume (TrV).

32. Method according to one of the claims 24 to 31, wherein the fluid flow is directed retrograde relative to the natural body fluid flow direction in the conduit (V) of the body fluid circuit (BC), and/or
wherein during a first treatment step the fluid flow is directed into a first flow direction within the transport volume (TrV) and wherein during a second treatment step that follows after or is before the first treatment step the fluid flow within the transport volume (TrV) is directed into a second flow direction that is a reverse direction relative to the first flow direction.

33. Method according to one of the claims 24 to 32, wherein the border is formed by a border element that comprises an expandable arrangement,
wherein the expandable arrangement has a non-expanded state and an expanded state,
wherein the expandable arrangement can be switched from the non-expanded state to the expanded state.

34. Method according to claims 33, wherein the expandable arrangement comprises at least one balloon (B a) that is inflatable using a liquid fluid or a gaseous fluid, and/or
wherein the balloon (Ba) has an axial length along a longitudinal axis of the cannula of at least 5 mm, at least 10 mm, at least 15 mm or of at least 20 mm

35. Method according to claim 34, wherein the cannula comprises a further lumen (CH1, CH2) that guides a fluid into and/or out of the balloon (B a), and
wherein the further lumen (CH1, CH2) is arranged separate from the inner lumen and extends from the proximal end of the cannula (L17) to the balloon (Ba).

36. Method according to claim 33, wherein the expandable arrangement comprises a plurality of wires, preferably at least two, at least three or at least four wires (1612), and
wherein the expandable arrangement comprises at least one membrane (949, 919) that is connected to at least two of the wires (1612) of the plurality of wires,
wherein the membrane defines an opening that faces laterally and wherein the membrane is positioned such that the opening faces to at least one or to both of the right pulmonary veins (rPV) and wherein after the positioning of the membrane a blood flow from at least one or from both of the left pulmonary veins (IPV) to the left ventricle (LV) is possible.

37. Method according to one of the claims 24 to 36, wherein the fluid flow is guided through the cannula (L1b) from outside of the body (100) of a subject into the body (100) of the subject, or wherein the fluid flow is guided through the cannula (L1b) out of the body (100) of the subject.

38. Method according to one of the claims 24 to 37, wherein the fluid flow comprises and/or consists to at least 20 percent or at least 60 percent or at least 80 percent of volume of blood or of components of blood or a fluid that does not comprise blood or blood components.

39. Method according to one of the claims 24 to 38, wherein the fluid flow comprises at least one medicament and/or treatment substance for treating lung (L) cancer or a lung (L) infection or for cleaning the lung (L).

40. Method according to one of the claims 24 to 39, wherein a treatment substance is applied by inhalation in addition to the fluid flow through the transport volume (TrV) simultaneously to the fluid flow, within 24 hours before or after the perfusion of the transport volume (TrV) or within one week before or after the perfusion of the transport volume (TrV) by the fluid flow.

41. Method according to one of the claims 24 to 40, wherein a radioactive treatment, preferably radioactive oncology or radio-oncology treatment, is applied in addition to the fluid flow through the transport volume (TrV) simultaneously to the fluid flow, within the same day, within the same week or within the same month as the fluid flow.

42. Method according to one of the claims 39 to 41, wherein a treatment substance is applied using nano particles or micro particles.

43. Method according to one of the claims 24 to 42, wherein the fluid flow is guided out of the body (100) of a subject using the cannula (L1b) or a further cannula (L2b),
wherein the fluid flow is extracorporeal cleaned and/or filtered using a filter unit (ADSb), especially using a filter unit (ADSb) that comprises at least one a fluid filter and/or at least one adsorption element and/or at least one absorption element and/or at least one permeable or semipermeable membrane for filtering and/or dialyzing element,
and wherein the cleaned and/or filtered fluid flow is used again to be guided through the transport volume (TrV).

44. Method according to one of the claims 24 to 43 wherein the fluid flow is guided out of the body (100) of a subject using the cannula (L1b),
wherein the oxygen contents of the fluid flow is increased or decreased extracorporeally and/or wherein the contents of carbon dioxide is increased or decreased, and
wherein the fluid flow that has a changed content of oxygen and/or carbon dioxide is used to be guided through the transport volume (TrV).

45. Method according to one of the claims 24 to 44, wherein only one pump (Pb, Pc, Pd) is used for driving the fluid flow through the transport volume (TrV) and for at least one of draining out body fluid and deliver cleaned/or oxygenated body fluid into the body (100) of a subject.

46. Method according to one of the claims 24 to 45, wherein a first pump (Pe1) is used for driving the fluid flow through the transport volume (TrV), and
wherein a second pump (Pe2) is used for the delivery of oxygenated blood into the body and/or for drainage of body fluid out of the body of a subject,
wherein at least one of the pumps (Pe1, Pe2) generates a pulsed fluid flow or wherein both of the pumps (Pe1, Pe2) generate a pulsed fluid flow.

47. Method according to one of the claims 24 to 46, wherein a first pump (Pe1) is used for driving the fluid flow through the transport volume, and
wherein a second pump (Pe2) is used for the delivery of oxygenated blood into the body and/or for drainage of body fluid out of the body of an subject,
wherein at least one of the pumps (Pe1, Pe2) generates a continuous fluid flow or wherein both of the pumps (Pe1, Pe2) generate a continuous fluid flow.

48. Method according to one of the claims 24 to 47, wherein the cannula (L1b) is a delivery cannula that is used to guide the fluid flow out of at least one opening of the delivery cannula into the transport volume (TrV), and
wherein a drainage cannula is used to drain the fluid flow from the transport volume (TrV) using at least one opening of the drainage cannula, and
wherein both cannulas isolate the transport volume (TrV) from the body fluid circuit (BC).

49. Method according to one of the claims 24 to 47, wherein the cannula (L1b) is a drainage cannula that is used to drain the fluid flow from the transport volume (TrV) using at least one opening of the drainage cannula, and
wherein a delivery cannula is used to guide the fluid flow out of at least one opening of the delivery cannula into the transport volume (TrV),
wherein both cannulas isolate the transport volume (TrV) from the body fluid circuit (BC).

50. Method according to one of the claims 24 to 49, wherein the cannula (L1b) is inserted jugularly, and wherein a second cannula that is used to guide the fluid flow is also inserted jugularly.

51. Method according to one of the claims 24 to 50, wherein the cannula (L2b) is guided at least partially through at least one chamber of a heart into both left pulmonary veins (IPV) or into both right pulmonary veins (rPV).

52. Method according to one of the claims 24 to 50, wherein the cannula (L1b) is guided at least partially through the right atrium (RA) and/or through the right ventricle (RV) and into the main pulmonary artery (PA), the left pulmonary artery (IPA) or the right pulmonary artery (rPA).

53. Method according to one of the claims 24 to 52, wherein the cannula (L2b) is a single lumen cannula.

54. Method according to one of the claims 24 to 52, wherein the cannula (L1c) is a dual-lumen cannula (DLC) or a multi-lumen cannula.

55. Method according to claim 54, wherein the cannula is a fixed dual-lumen or multi-lumen cannula (DLc), wherein at least two lumens of the cannula are not axially movably with regard to each other.

56. Method according to claim 54, wherein the cannula is a non-fixed dual-lumen or multi-lumen cannula (DLc), wherein at least two lumens of the cannula are axially movably with regard to each other during insertion of the cannula into a body of a subject.

57. Method according to one of the claims 24 to 56, wherein the transport volume (TrV) is limited to only one organ or to only a part of an organ, preferably to the liver or the kidneys or the stomach or the liver or the brain or an organ of the digestive system, preferably the colon or the gall bladder or the urinary bladder or the heart or the pancreas.

58. Method according to one of the claims 24 to 57, wherein a cannula (L1b, L2b) according to one of the claims 1 to 9 is used and/or wherein a cannula system (L1b, L2b; L1c, L3c) according to one of the claims 10 to 18 is used and/or wherein a kit according to one of the claims 19 to 23 is used.

59. Method according to any one of the claims 24 to 58 wherein a treatment substance (M) is applied through at least substance transport channel into a selected region of the lung (L) simultaneously to the fluid flow through the transport volume (TrV).

60. Method for treating the lung (L),
transporting a treatment substance (M) through at least one substance transport channel into a selected region of the lung (L) to treat the selected region,
while treating the selected region transporting a liquid through a transport volume (TrV) which is separated from a body fluid circuit (BC),
wherein the selected region is associated with the transport volume (TrV).

61. Method according to claim 60, wherein the transport volume (TrV) is limited by at least one semipermeable membrane of the lung (L), and/or
wherein the transport volume (TrV) comprises tissue of alveoli adjacent to a semipermeable membrane of alveoli of the lung (L)

62. Method according to claim 60 or 61, wherein the at least one substance transport channel is arranged within at least one air transport channel of the lung (L).

63. Method according to any one of the claims 60 to 62, comprising:
while treating and/or while transporting the treatment substance (M) through the at least one substance transport channel transporting the liquid through the transport volume (TrV), and
while treating and/or transporting the treatment substance (M) through the at least one substance transport channel transporting the liquid to the outside of a body of which the lung (L) is part of.

64. Method according to any one of the claims 60 to 63, wherein the treatment substance (M) is transported to only one half (lH, rH) of the lung (L), to only one lobe (Lo1 to Lo5) of the lung (L) or to only a part of a lobe (Lo1 to Lo5) of the lung (L).

65. Method according to claim 64, wherein
a) the treatment substance (M) is transported only to a part or to all alveoli in one selected half (lH, rH) of the lung (L) but not to non-selected alveoli in a non-selected half (lH, rH) of the lung (L) and wherein the liquid is transported in an essentially closed or in a closed transport volume (TrV) at least through the tissue of the selected alveoli in the selected half (lH, rH) of the lung (L) but not to alveoli in the non-selected half (lH, rH) of the lung (L), or
b) the treatment substance (M) is transported only to alveoli in one selected lobe (Lo1 to Lo5) of a selected half (lH, rH) of the lung (L) but not to non-selected lobes (Lo1 to Lo5) of the selected half (lH, rH) of the lung (L) or only to alveoli in some selected lobes (Lo1 to Lo5) of the selected half (lH, rH) of the lung (L) but not to a non-selected lobe (Lo1 to Lo5) in the selected half (lH, rH) of the lung (L) and wherein the liquid is transported in an essentially closed or in closed transport volume (TrV) at least through the tissue of the alveoli in the selected lobe (Lo1 to Lo5) or in the selected lobes (Lo1 to Lo5) but not through the tissue of alveoli in a non-selected half (lH, rH) of the lung (L) or in the non-selected lobes (Lo1 to Lo5) of the lung (L), or
c) the treatment substance (M) is transported only to a first part of the alveoli of a selected lobe (Lo1 to Lo5) of a selected half (lH, rH) of the lung (L) but not to a second part of alveoli of the selected lobe (Lo1 to Lo5) and not to alveoli in a non-selected half (lH, rH) of the lung (L) and wherein the liquid is transported in an essentially closed or in a closed transport volume (TrV) at least through the tissue of the first part of the alveoli in the selected half (lH, rH) of the lung (L) but not through the tissue of alveoli in the non-selected half (lH, rH) of the lung (L) of through the tissue of alveoli in the non-selected lobes (Lo1 to Lo5) of the lung (L).

66. Method according to any one of the claims 60 to 65, using a cannula (L1b, L2b) according to any one of the claims 1 to 9 or a cannula system (L1b, L2b; L1c, L2c) according to any one of the claims 10 to 18 for transporting the liquid or using a method according to any one of the claims 24 to 58.

67. Method according to any one of the claims 60 to 66, wherein the treatment substance (M) comprises stem cells of at least one kind of tissue in the lung (L) or a medicament against lung (L) cancer or a medicament against a lung (L) infection.

68. Method according to any one of the claims 60 to 67, wherein the treatment substance (M) or at least a part of the treatment substance (M) is removed from the liquid and wherein the liquid is transported back into the body, preferably back into the transport volume (TrV).

69. Kit for defining at least one border of a transport volume (TrV) for an in-vivo fluid transport, comprising:
a cannula (L1b, L2b) according to any one of the claims 1 to 9, and/or
a cannula system (L1b, L2b; L1c, L3c) according to any one of the claims 10 to 18, and:
a transport device (Tu, INH) comprising at least one substance transport channel (Tu) for transporting a treatment substance (M) preferably to only a selected region of the lung (L),
wherein preferably the substance transport channel (Tu) is adapted to be arranged at least partially or at least with 90 percent of its length in an air transport channel (Tr) of the lung (L),
wherein the transport device (Tu, INH) has at least one port outside of the lung (L) and at least one port inside of the lung (L).

70. Kit according to claim 69, comprising at least one pump (Pb) for driving a fluid flow through the transport volume (TrV), preferably a roller pump or a membrane pump or a centrifugal pump or a diagonal pump or an axial pump, and/or
a delivery unit (C1b, C2) for the introduction of at least one treatment substance (M) into the fluid flow in order to guide the treatment substance (M) and/or further treatment substance (M) into the transport volume (TrV) via the fluid flow, and/or
a removing device for removing the treatment substance (M) and/or the further treatment substance (M) from the fluid flow.

71. Kit according to claim 69 or 70, wherein the kit is used for performing a method according to any one of the claims 60 to 68.
